(19) United States
(12) Patent Application Publication
(10) Pub. No.: US 2004/0110761 A1
(43) Pub. Date: Jun. 10, 2004

(54) Combination therapy for the prophylaxis and treatment of hyperlipidemic conditions and disorders

(75) Inventors: Bradley T. Keller, Chesterfield, MO (US); Samuel J. Tremont, St. Louis, MO (US); Kevin C. Glenn, Maryland Heights, MO (US); Robert E. Manning, St. Louis, MO (US)

Correspondence Address: BANNER & WITCOFF, 1001 G STREET N W, SUITE 1100, WASHINGTON, DC, 20001, US

(73) Assignee: G.D. SEARLE LLC, Chicago, IL

(21) Appl. No.: 10/611,942

(22) Filed: Jul. 3, 2003

Related U.S. Application Data
(63) Continuation of application No. 09/802,313, filed on Mar. 8, 2001, now abandoned.
(60) Provisional application No. 60/188,378, filed on Mar. 10, 2000.
(60) Provisional application No. 60/188,361, filed on Mar. 10, 2000.

(51) Int. Cl. A61K 31/498; A61K 31/401; A61K 31/366; A61K 31/225

(52) U.S. Cl. 514/249; 514/423; 514/460; 514/548

(57) ABSTRACT

Novel methods and combinations for the treatment and/or prophylaxis of a hyperlipidernic condition or disorder in a subject, wherein the methods comprise the administration of one or more HMG Co-A reductase inhibitors and one or more ASBT inhibitors selected from the specific group of compounds described herein, and the combinations comprise one or more HMG Co-A reductase inhibitors and one or more of said apical sodium co-dependent bile acid transport inhibitors.

### CROSS-REFERENCE TO RELATED APPLICATIONS

This application claims pnority from U.S. Provisional Application Serial No. 60/188,378 filed Mar. 10, 2000, and from U.S. Provisional Application Serial No. 60/188,361 filed Mar. 10, 2000.

This application is being simultaneously filed with a related application entitled “Method For The Preparation Of Tetrahydrobenzothiepines”, Serial No. ______ The contents of this related patent application are incorporated herein by reference as if fully set forth at length.

### BACKGROUND OF THE INVENTION

1. Field of the Invention

The present invention relates to methods for the treatment and/or prophylaxis of hyperlipidemic conditions and/or disorders in a subject, and specifically relates to combinations of compounds, pharmaceutical compositions comprising such combinations, and methods for their use in medicine. More particularly, the present invention relates to apical sodium co-dependent bile acid transport inhibitors and 3hydroxy-3-methylglutaryl coenzyme-A reductase inhibitors.

2. Description of the Related Art

The major metabolic fate of cholesterol in the human body is in the hepatic synthesis of bile acids. Bile acids are both passively and actively reabsorbed from the small intestine and recycled via the enterohepatic circulation to conserve the total pool of bile acids. Dietschy, “Mechanisms for the intestinal absorption of bile acids”, J. Lipid Res., 9:297-309 (1968). Bile acids undergo passive absorption in the proximal small intestine and active transport in the terminal ileum. Love et al., “New insights into bile acid transport”, Curr. Opin. Lipidol., 9(3):225-229 (1998). Ileal active transport accounts for the majority of intestinal bile acid uptake and is the exclusive route for taurine-conjugated conjugated bile acids. Id. Ileal active transport is mediated by the apical sodium co-dependent bile acid transporter (“ASBT”, also known as the ileal bile acid transporter or “IBAT”) localized to the distal one-third of the ileum. Craddock et al., “Expression and transport properties of the human ileal and renal sodium-dependent bile acid transporter”, Am. J. Physiol., 274 (Gastrointest. Liver Physiol. 37):G157-G169 (1998).

An equilibrium generally exists between hepatic cholesterol and the bile acid pool. Interruption of the enterohepatic recirculation of bile acids (e.g., the binding of intestinal bile acids to a sequestering resin such as cholestyramine; the surgical removal of the ileum to physically eliminate ileal ASBT; or the specific inhibition of ileal ASBT) results in a decrease in the liver bile acid pool and stimulates increased hepatic synthesis of bile acids from cholesterol (i.e., an upregulation of cholesterol-7α-hydroxylase activity), eventually depleting the liver's pool of esterified cholesterol. In order to maintain liver cholesterol levels necessary to support bile acid synthesis, the de novo synthesis of cholesterol increases in the hepatocytes (i.e., an upregulation of 3-hydroxy-3-methylglutaryl coenzyme-A reductase activity) and also increases the uptake of serum cholesterol by upregulating the number of cell surface low density lipoprotein cholesterol receptors (“LDL receptors”). The number of hepatic LDL receptors directly impacts serum low density lipoprotein (“LDL”) cholesterol levels, with an increase in the number of LDL receptors resulting in a decrease in serum cholesterol. The net result, therefore, is that serum LDL cholesterol levels decrease when intestinal bile acid reabsorption is reduced.

A class of antihyperlipidemic agents that operates by inhibiting bile acid reabsorption in the ileum recently has been identified. Examples of this class of agents include the substituted benzothiepines disclosed in U.S. Pat. No. 5,994,391. PCT Patent Application No. WO99/35135 discloses additional substituted benzothiazepine compounds for use as ASBT inhibitors. By way of further example, PCT Patent Application No. WO94/24087 discloses a group of substituted naphthalene compounds for use as ABST inhibitors. The United States Food and Drug Administration, however, has not approved any ASBT inhibitor for use as an antihyperlipidemic agent at this time.

Numerous antihyperlipidemic agents having other modes of action also have been disclosed in the literature as useful for the treatment of hyperlipidemic conditions and disorders. These agents include, for example, commercially available drugs such as nicotinic acid, bile acid sequestrants including cholestryramine and colestipol, 3-hydroxy-3-methylglutaryl coenzyme-A reductase inhibitors (“HMG Co-A reductase inhibitors”), probucol, and fibric acid derivatives including gemfibrozil and clofibrate.

The class of antihyperlipidemic agents known as HMG Co-A reductase inhibitors operates by inhibiting the hepatic enzyme 3-hydroxy-3-methylglutaryl coenzyme-A reductase (“HMG Co-A reductase”). Direct inhibition of HMG Co-A reductase by the monotherapeutic administration of HMG Co-A reductase inhibitors such as pravastatin has been shown to be a clinically effective method of lowering serum LDL cholesterol. Sacks et al., “The Effect of Pravastatin on Coronary Events after Myocardial Infarction in Patients with Average Cholesterol Levels”, New England Journal of Medicine, 335(14):1001-9 (1996). Monotherapeutic treatment with pravastatin may lead to upregulation of cell surface LDL receptors as a mechanism to provide cholesterol to the liver in support of bile acid synthesis. Fujioka et al., “The Mechanism of Comparable Serum Cholesterol Lowering Effects of Pravastatin Sodium, a 3-Hydroxy-3-Methylglutaryl Coenzyme A Inhibitor, between Once- and Twice-Daily Treatment Regimens in Beagle Dogs and Rabbits”, Jpn. J. Pharmacol., Vol. 70, pp. 329-335 (1996).

The administration of an ASBT inhibitor in combination with an HMG Co-A reductase inhibitor is generally disclosed in PCT Application WO98/40375.

The treatment of hypercholesterolemia with an HMG Co-A reductase inhibitor in combination with a bile acid sequestering resin also has been reported in the literature. The administration of the HMG Co-A reductase inhibitor lovastatin in combination with the bile acid sequestering resin colestipol is disclosed in Vega et al., “Treatment of Primary Moderate Hypercholesterolemia With Lovastatin (Mevinolin) and Colestipol”, JAMA, Vol. 257(1), pp. 33-38 (1987). The administration of the HMG Co-A reductase inhibitor pravastatin in combination with the bile acid sequestering resin cholestyramine is disclosed in Pan et al., “Pharmacokinetics and pharmacodynamics of pravastatin alone and with cholestyramine in hypercholesterolemia”, Clin. Pharmacol. Ther., Vol. 48, No. 2, pp. 201-207 (August 1990).

The treatment of hypercholesterolemia with other selected combination regimens also has been reported in the literature. Ginsberg, “Update on the Treatment of Hypercholesterolemia, with a Focus on HMG Co-A Reductase Inhibitors and Combination Regimens”, Clin. Cardiol., Vol. 18(6), pp. 307-315 (June 1995), reports that, for resistant cases of hypercholesterolernia, therapy combining an HMG Co-A reductase inhibitor with either a bile acid sequestering resin, niacin or a fibric acid derivative generally is effective and well tolerated. Pasternak et al., “Effect of Combination Therapy with Lipid-Reducing Drugs in Patients with Coronary Heart Disease and ‘Normal’ Cholesterol Levels”, Annals of Internal Medicine, Vol. 125, No. 7, pp. 529-540 (October 1, 1996) reports that treatment with either a combination of the HMG Co-A reductase inhibitor pravastatin and nicotinic acid or a combination of pravastatin and the fibric acid derivative gemfibrazol can be effective in lowering LDL cholesterol levels.

The novel combinations of the present invention, however, exhibit improved efficacy, improved potency, and/or reduced dosing requirements for the active compounds relative to the specific combination regimens previously disclosed in the published literature.

### SUMMARY OF THE INVENTION

Among the various aspects of the invention are methods for the treatment and/or prophylaxis of a hyperlipidemic condition and/or disorder in a subject comprising the administration of one or more HMG Co-A reductase inhibitors and one or more ASBT inhibitors selected from the group consisting of compounds A-1 through A-5 and A-6 through A-15 as further described below.

The invention is further directed to combinations, including pharmaceutical compositions, comprising one or more HMG Co-A reductase inhibitors and one or more ASBT inhibitors selected from the group consisting of compounds A-1 through A-5 and A-6 through A- 15 as further described below.

The invention is further directed to kits comprising one or more HMG Co-A reductase inhibitors and one or more ASBT inhibitors selected from the group consisting of compounds A-1 through A-5 and A-6 through A-15 as further described below.

The invention is further directed to the compound having the formula

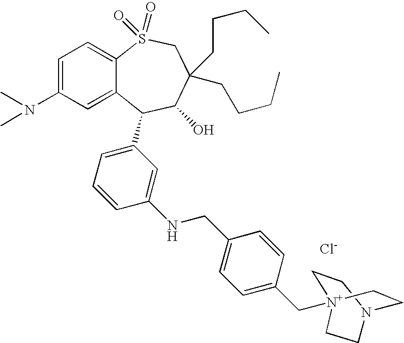

and the pharmaceutically acceptable salts, esters and prodrugs thereof.

Other aspects of the invention will be in part apparent and in part pointed out hereinafter.

### BRIEF DESCRIPTION OF THE DRAWINGS

FIG. 1 shows typical X-ray powder diffraction patterns for Form I (plot (a)) and Form II (plot (b)) of compound 41 of the working examples.

FIG. 2 shows typical Fourier transform infrared (FTIR) spectra for Form I (plot (a)) and Form II (plot (b)) of compound 41 of the working examples.

FIG. 3 shows typical solid state carbon-13 nuclear magnetic resonance (NMR) spectra for Form I (plot (a)) and form II (plot (b)) of compound 41 of the working examples.

FIG. 4 shows typical differential scanning calorimetry profiles for Form I (plot (a)) and Form II (plot (b)) of compound 41 of the working examples.

FIG. 5 shows water sorption isotherms for Form I (plot (a)) and Form II (plot (b)) of compound 41 of the working examples.

### DETAILED DESCRIPTION OF THE PREFERRED EMBODIMENTS

It has been discovered that the administration to a subject of one or more ASBT inhibitors selected from the specific group consisting of compounds A-1 through A-5 and A-6 through A-15 as described below, and one or more HMG Co-A reductase inhibitors provides improved results in the prophylaxis and/or treatment of hyperlipidemic conditions and/or disorders relative to other combination regimens, particularly improved efficacy, improved potency, and/or reduced dosing requirements for the active compounds. The method comprises administering a first amount of the ASBT inhibitor and a second amount of the HMG Co-A reductase inhibitor wherein the first and second amounts of the inhibitors together comprise a therapeutically effective amount of the inhibitors for the prophylaxis and/or treatment of hyperlipidemic conditions and/or disorders.

The term “hyperlipidemic condition and/or disorder” is used broadly in this application and encompasses, for example, dyslipidemic conditions and/or disorders generally as well as pathological conditions and/or disorders in a subject caused or exacerbated by a dyslipidemic condition or disorder. Such pathological condition or disorder may exist as a continuous or chronic condition or occur intermittently or acutely in a subject. Typical dyslipidemic conditions and disorders include, but are not limited to, hyperlipidemia, hypercholesterolemia, hypertriglyceridemia, hyperlipoproteinemia, hyperbetalipoproteinemia (high LDL), hyperprebetalipoproteinemia (high VLDL), hyperchylomicronemia, hypolipoproteinemia, and hypoalphalipoproteinemia (low HDL).

Although dyslipidemic conditions and disorders generally are characterized based on the presence of “hyper-” (elevated) or “hypo-” (diminished) amounts of particular lipids or lipoproteins, such terms are relative terms with regard to the potential of a “hyper-” or “hypo-” dyslipidemia to cause or exacerbate a pathological condition. Thus, for example, absolute values of these molecules, which may be expressed in units of concentration, such as mg/dl or mmol/l in the circulation, may fluctuate over a wide range and, depending on individual factors, such as genetic traits and life-style habits, may cause or exacerbate a pathological condition and/or disorder at a concentration similar to what would be considered normolipidemic, by one skilled in the art.

Illustrative pathological conditions and/or disorders that may be caused or exacerbated by a dyslipidemic condition include, but are not limited to cardiovascular diseases; atherosclerosis; arteriosclerosis; myocardial infarction; stroke; hyper-thrombotic conditions; vascular dysfunction; endothelial dysfunction; heart failure; arrhythmia; inflammation of cardiovascular tissues such as heart, valves, vasculature, arteries and veins; aneurysms; stenosis; restenosis; vascular plaques; vascular fatty streaks; leukocyte, monocyte and/or macrophage infiltrate; intimal thickening; medial thinning; infectious and surgical trauma; and vascular thrombosis.

ASBT Inhibitors

The ASBT inhibitor is selected from the group of ASBT inhibitors disclosed in Table 1, including the diastereomers, enantiomers, racemates, salts, tautomers, conjugate acids, and prodrugs of those ASBT inhibitors.

TABLE 1

| Compound | |
|---|---|
| Number | Structure |
| A-1 | 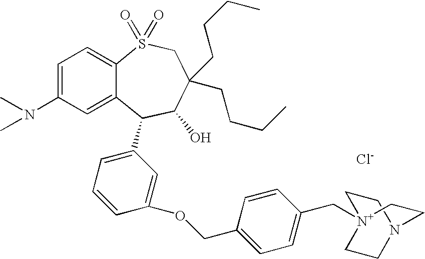 |
| A-2 | 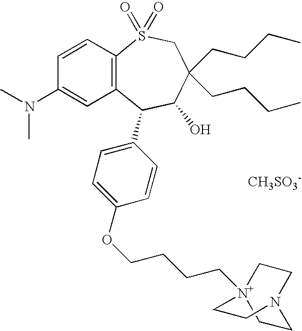 |
| A-3 | 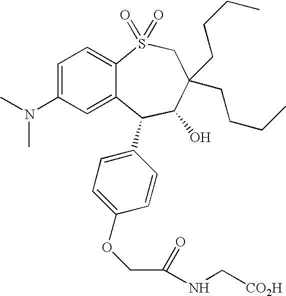 |
| A-4 | 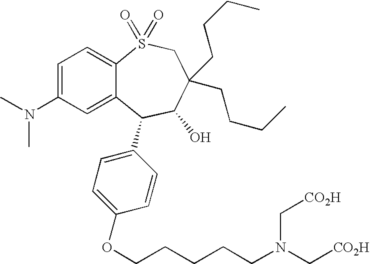 |
| A-5 | 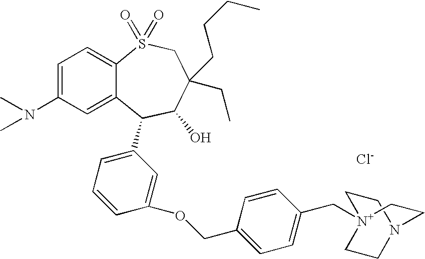 |
| A-7 | 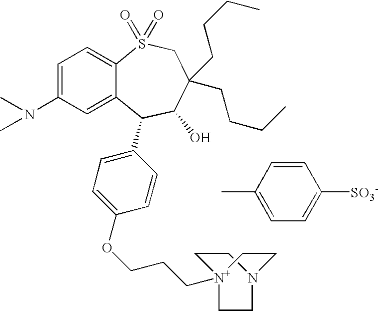 |
| A-8 | 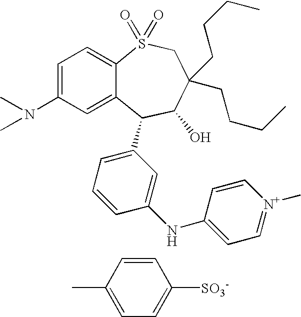 |
| A-9 | 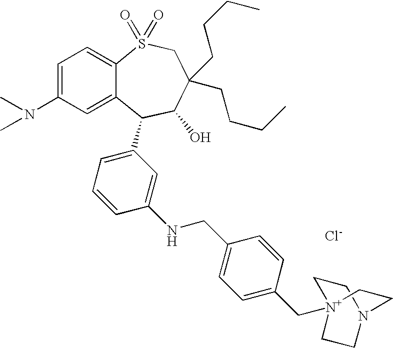 |
| A-10 | 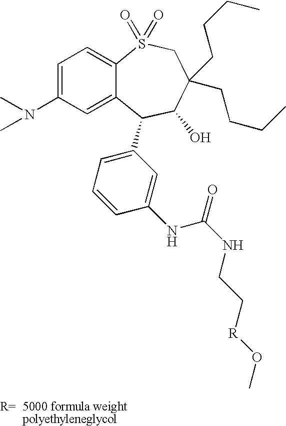 |
| A-11 | 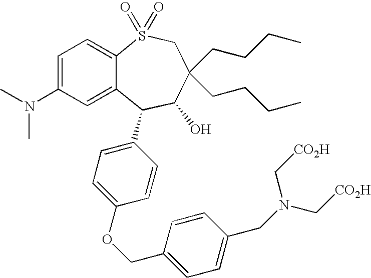 |
| A-12 | 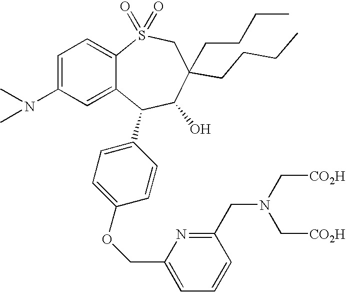 |
| A-13 | 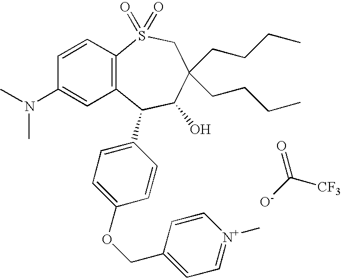 |
| A-14 | 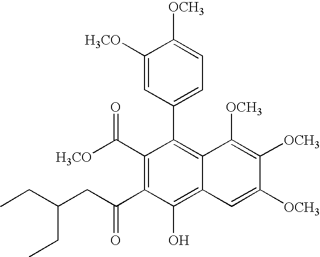 |
| A-15 | 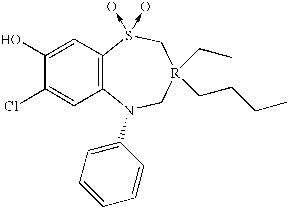 |

The individual patent documents referenced in Table 2 below describe the preparation of the ASBT inhibitors of Table 1 and are each herein incorporated by reference.

TABLE 2

| Compound | Patent/Literature Reference for Preparation of |
|---|---|
| Number | Compound Per Se |
| A-1 | SEE WORKING EXAMPLES 14, 29, 29A, 30 AND 30A; |
| | ALSO SEE U.S. Pat. No. 5,994,391: EXAMPLE 1426 |
| | and EXAMPLE 1426a |
| A-2 | U.S. Pat. No. 5,994,391: EXAMPLE 1408 |
| A-3 | U.S. Pat. No. 5,994,391: EXAMPLE 1403 |
| A-4 | U.S. Pat. No. 5,994,391: EXAMPLE 1415 |
| A-5 | SEE WORKING EXAMPLES 14, 29, 29A, 30 AND 30A; |
| | ALSO SEE U.S. Pat. No. 5,994,391: EXAMPLE 1426 |
| | and EXAMPLE 1426a |
| A-7 | U.S. Pat. No. 5,994,391: EXAMPLE 1407 |
| A-8 | U.S. Pat. No. 5,994,391: EXAMPLE 1450 |
| A-9 | SEE WORKING EXAMPLE 16 |
| A-10 | U.S. Pat. No. 5,994,391: EXAMPLE 1455 |
| A-11 | U.S. Pat. No. 5,994,391: EXAMPLE 1427 |
| A-12 | U.S. Pat. No. 5,994,391: EXAMPLE 1431 |
| A-13 | U.S. Pat. No. 5,994,391: EXAMPLE 1428 |
| A-14 | WO94/24087 |
| A-15 | WO99/35135 |

In another embodiment, the ASBT inhibitor is selected from the group consisting of Compounds A-1 through A-5 and A-7 through A-13.

In another embodiment, the ASBT inhibitor is selected from the group consisting of Compounds A-1, A-2, A-5, A-7, A-8, A-9, and A-13.

In another embodiment, the ASBT inhibitor is selected from the group consisting of Compounds A-3, A-4, A-11 and A-12.

In another embodiment, the ASBT inhibitor is Compound A-10.

In another embodiment, the ASBT inhibitor is Compound A-5.

In another embodiment, the ASBT inhibitor is Compound A-1. Compound A-1 can be present, for example, in the form of the (4R,5R) enantiomer, the (4S,5S) enantiomer, or racemic or other combinations thereof. Preferably, Compound A-1 is present in the form of the (4R,5R) enantiomer, also known as (4R,5R)-1-((4-(4-(3,3dibutyl-7-(dimethylamino)-2,3,4,5-tetrahydro-4-hydroxy-1,1 -dioxido-1-benzithiepin-5yl)phenoxy)methyl)phenyl)methyl4-aza-1-azoniabicyclo[2.2.2]octane chloride.

The HMG Co-A Reductase Inhibitor

HMG Co-A reductase inhibitors encompassing a wide range of structures are useful in the combinations and methods of the present invention. Such HMG Co-A reductase inhibitors may be, for example, statins that have been synthetically or semi-synthetically prepared, statins extracted from natural sources such as plants, or statins isolated as fungal metabolites from cultures of suitable microorganisms. Nonlimiting examples of HMG Co-A reductase inhibitors that may be used in the present invention include those HMG Co-A reductase inhibitors disclosed in Table 3, including the diastereomers, enantiomers, racemates, salts, tautomers, conjugate acids, and prodrugs of the HMG Co-A reductase inhibitors of Table 3. The therapeutic compounds of Table 3 can be used in the present invention in a variety of forms, including acid forn, salt form, racemates, enantiomers, zwitterions, and tautomers.

TABLE 3

| | CAS NUMBERS FOR | |
|---|---|---|
| COMPOUNDS AND | SPECIFIC AND | |
| COMPOUND | REPRESENTATIVE | |
| CLASSES | COMPOUDS | REFERENCE |
| Benfluorex | 23602-78-0 | ES 474498, Servier |
| Fluvastatin | 93957-54-1 | EP 244364, Sandoz |
| Lovastatin | 75330-75-5 | EP 22478, Merck & Co. |
| Pravastatin | 81093-37-0 | DE 3122499, Sankyo |
| Simvastatin | 79902-63-9 | EP 33538, Merck & Co. |
| Atorvastatin | 134523-00-5 | EP 409281, Warner- |
| | | Lambert |
| Cerivastatin | 145599-86-6 | JP 08073-432, Bayer |
| Bervastatin and related | 132017-01-7 | EP 380392, Merck |
| benzopyrans | | KGaA |
| ZD-9720 | | WO97/06802 |
| ZD-4522 (also called | 147098-20-2 (calcium salt); | EP 521471; |
| Rosuvastatin) | 147098-18-8 (sodium salt) | Bioorg. Med. Chem., |
| | | Vol. 5(2), pp. 437-444 |
| | | (1997); Drugs Future, |
| | | Vol. 24(5), pp. 511-513 |
| | | (1999) |
| BMS 180431 | 129829-03-4; | Sit, Parker, Motoc, Han, |
| | 157243-11-3 | Balasubramanian, Catt, |
| | | Brown, Harte, |
| | | Thompson, and Wright, |
| | | J. Med. Chem., (1990), |
| | | 33(11), 2982-99; Bristol- |
| | | Myers Squibb |
| NK-104 (also called | 141750-63-2 | Takano, Kamikubo, |
| pitavastatin and | | Sugihara, Suzuk, |
| nisvastatin) | | Ogasawara, Tetahedron: |
| | | Assymetry, (1993), 4(2), |
| | | 201-4; Nissan Chemical |
| SR-12313 | 126411-39-0 | SmithKline Beecham |
| Carvastatin | 125035-66-7 | Tobishi Yakuhin Kogyo |
| | | Co. Ltd. |
| PD-135022 | 122548-95-2 | Parke-Davis & Co. |
| Crilvastatin | 120551-59-9 | Pan Medica |
| (Carboxydihydroxy- | 148966-78-3, 139993-44-5, | EP 464845; Shionogi |
| heptenyl)- | 139993-45-6, 139993-46-7, | |
| sulfonylpyrroles | 139993-47-8, 139993-48-9, | |
| including S-4522 | 139993-49-0, 139993-50-3, | |
| | 139993-51-4, 139993-52-5, | |
| | 139993-53-6, 139993-54-7, | |
| | 139993-55-8, 139993-56-9, | |
| | 139993-57-0, 139993-58-1, | |
| | 139993-59-2, 139993-60-5, | |
| | 139993-61-6, 139993-62-7, | |
| | 139993-63-8, 139993-64-9, | |
| | 139993-65-0, 139993-66-1, | |
| | 139993-67-2, 139993-68-3, | |
| | 139993-69-4, 139993-70-7, | |
| | 139993-71-8, 139993-72-9, | |
| | 139993-73-0, 139993-74-1, | |
| | 139993-75-2, 139993-76-3, | |
| | 139993-77-4, 139993-78-5, | |
| | 139993-79-6, 139993-80-9, | |
| | 140110-63-0, 140128-98-9, | |
| | 140128-99-0, 140157-62-6 | |
| Boron analogs of di- and | 125894-01-1, 125894-02-2, | Sood, Sood Spielvogel, |
| tripeptides | 125894-03-3, 125894-04-4, | Hall, Eur. J. Med. Chem., |
| | 125894-05-5, 125894-08-8, | (1990), 25(4), 301-8; |
| | 125894-09-9, 125914-96-7 | Boron Biologicals |
| Zaragozic Acids | 157058-13-4, 157058-14-5, | GB 2270312 |
| | 157058-15-6, 157058-16-7, | |
| | 157058-17-8, 157058-18-9, | |
| | 157058-19-0 | |
| Seco-oxysterol analogs | 157555-28-7, 157555-29-8 | Larsen, Spilman, Yagi, |
| including U-88156 | | Dith, Hart and Hess, J. |
| | | Med. Chem., (1994), |
| | | 37(15), 2343-51; |
| | | Pharmacia & Upjohn |
| U-9888; U-20685; U- | 39945-32-9 | Pharmacia and Upjohn |
| 51862; and U-71690 | | |
| Pyridopyrimidines | 64405-40-9, | Hermecz, Meszaros, |
| including acitemate | 101197-99-3 | Vasvari-Debreczy, |
| | | Hovarth, Virag, and |
| | | Sipos, Hung. Arzneim- |
| | | Forsch., (1979), 29(12), |
| | | 1833-5; Mitsubishi |
| | | University |
| BMY 22566 | 129829-03-4 | Sit, Parker, Motoc, Han, |
| | | Balasubramanian, Catt, |
| | | Brown, Harte, |
| | | Thompson, and Wright, |
| | | J. Med. Chem., (1990), |
| | | 33(11), 2982-99 |
| Colestolone | 50673-97-7 | Raulston, Mishaw, Parish |
| | | and Schroepfer, |
| | | Biochem. Biophys. Res. |
| | | Commun., (1976), 71(4), |
| | | 984-9; American Home |
| | | Products |
| CP-83101 | 130746-82-6, 130778-27-7 | Wint and McCarthy, J. |
| | | Labelled Compd. |
| | | Radiopharm., (1988), |
| | | 25(11), 1289-97; Pfizer |
| Dalvastatin | 132100-55-1 | Kuttar, Windisch, Trivedi |
| | | and Golebiowski, J. |
| | | Chromatogr., A (1994), |
| | | 678(2), 259-63; Rhone- |
| | | Poulenc Rorer |
| Dihydromevinolin | 77517-29-4 | Falck and Yang, |
| | | Tetrahedron Lett., |
| | | (1984), 25(33), 3563-66; |
| | | Merck & Co. |
| DMP-565 | 199480-80-3 | Ko, Trzaskos, Chen, |
| | | Hauster, Brosz, and |
| | | Srivastava, Abstr. Papers |
| | | Am. Chem. Soc. (207^{th} |
| | | National Meeting, Part 1, |
| | | MEDI 10, 1994); Dupont |
| | | Merck |
| Pyridyl and Pyrimidinyl- | 122254-45-9 | Beck, Kessler, Baader, |
| ethenyldesmethyl- | | Bartmann, Bergmann, |
| mevalonates including | | Granzer, Jendralla, Von |
| glenvastin | | Kerekjarto, Krause, et al., |
| | | J. Med. Chem., (1990), |
| | | 33(1), 52-60; Hoechst |
| | | Marion Roussel |
| GR 95030 | 157243-22-6 | U.S. Pat. No. 5316765; Glaxo |
| | | Wellcome |
| Isoxazolopyridyl- | 130581-42-9, 130581-43-0, | EP 369323 |
| mevalonates, carboxylic | 130581-44-1, 130581-45-2, | |
| acids and esters | 130581-46-3, 130581-47-4, | |
| | 130581-48-5, 130581-49-6, | |
| | 130581-50-9, 130581-51-0, | |
| | 130581-52-1, 130619-07-7, | |
| | 130619-08-8, 130619-09-9 | |
| Lactones of 6-phenoxy- | 127502-48-1, 13606-66-1, | Jenderella, Granzer, Von |
| 3,5-dihydroxy-hexanoic | 136034-04-3 | Kerekjarto, Krause, |
| acids | | Schnacht, Baader, |
| | | Bartmann, Beck, |
| | | Bergmann, et al., J. Med. |
| | | Chem., (1991), 34(10), |
| | | 2962-83; Hoechst Marion |
| | | Roussel |
| L 659699 | 29066-42-0 | Chiang, Yang, Heck, |
| | | Chabala, and Chang, J. |
| | | Org. Chem., (1989), |
| | | 54(24), 5708-12; Merck |
| | | & Co. |
| L 669262 | 130468-11-0 | Stokker, J. Org. Chem., |
| | | (1994), 59(20). 5983-6; |
| | | Merck & Co. |
| Mevastatin | 73573-88-3 | JP 56051992; Sankyo |
| Pannorin | 137023-81-5 | Ogawa, Hasumi, Sakai, |
| | | Murzkwa and Endo, J. |
| | | Antibiot., (1991), 44(7), |
| | | 762-7; Toyoko Noko |
| | | University |
| Rawsonol | 125111-69-5 | Cane, Troupe, Chan, |
| | | Westley and Faulkner, |
| | | Phytochemistry, (1989), |
| | | 28(11), 2917-19; |
| | | SmithKline Beecham |
| RP 61969 | 126059-69-6 | EP 326386; Phone- |
| | | Poulenc Rorer |
| Bile Acid Derived HMG | | Kramer, Wess, Enhsen, |
| Co-A Reductase | | Bock, Falk, Hoffmann, |
| Inhibitors Including Na | | Neckermann, Grantz, |
| S-2467 and S-2468 | | Schulz, et al., Biochim. |
| | | Biophys. Acta D, (1994), |
| | | 1227(3), 137-54; Hoechst |
| | | Marion Roussel |
| SC 32561 | 76752-41-5 | U.S. Pat. No. 4230626; Monsanto |
| SC 45355 | 125793-76-2 | EP 329124; non- |
| | | industrial source |
| Phosphorus Containing | 133983-25-2 | U.S. Pat. No. 5274155; Bristol- |
| HMG Co-A Reductase | | Myers Squibb |
| Inhibitors Including SQ | | |
| 33600 | | |
| 6-Aryloxymethyl-4- | 135054-71-6, 136215-82-2, | EP 418648 |
| hydroxytetra-hydropyran- | 136215-83-3, 136215-84-4, | |
| 2-ones, carboxylic acids | 136215-85-5, 136315-18-9, | |
| and salts | 136315-19-0, 136315-20-3, | |
| | 136315-21-4, 136316-20-6 | |
| Atorvastatin calcium | 134523-03-8 | Baumann, Butler, |
| (CI 981) | | Deering, Mennen, Millar, |
| | | Nanninga, Palmer and |
| | | Roth, Tetrahedron Lett., |
| | | (1992), 33(17), 2283-4 |
| Mevinolin Analogs | | EP 245003 |
| Pyranone Derivatives | | U.S. Pat. No. 4937259 |
| 1,2,4-Triazolidine-3,5- | 16044-43-2 | WO 9000897 |
| diones | | |
| Isoazolidine-3,5-diones | 124756-24-7 | EP 321090 |
| CS-514 | 81181-70-6 | DE 3122499 |
| 1,10-bis(carboxy- | 32827-49-9 | DE 2038835 |
| methylthio)decane | | |
| α, β-, and γ- | | Huang and Hall, Eur. J. |
| alkylaminophenone | | Med. Chem., (1996), |
| analogs including N- | | 31(4), 281-90 |
| phenyl-piperazinopropio- | | |
| phenone | | |
| 3-Amino-1-(2,3,4- | | Huang and Hall, Arch. |
| mononitro-, mono- or | | Pharm., (1996), 329(7), |
| dihalophenyl)-propan-1- | | 339-346 |
| ones including 3- | | |
| morpholino-or | | |
| piperidino-1-(3- | | |
| nitrophenyl)-propan-1- | | |
| ones | | |
| Substituted isoxazolo | 64769-68-2 | U.S. Pat. No. 4049813 |
| pyridinones | | |
| Biphenyl derivatives | | JP 07O89898 |
| 4-[1-(Substituted | | Watanabe, Ogawa, Ohno, |
| phenyl)-2-oxo-pyrrolidin- | | Yano, Yamada and |
| 4-yl]methoxybenzoic | | Shirasaka, Eur. J. Med. |
| acids | | Chem., (1994), 29(9), |
| | | 675-86 |
| Dihydroxy(tetra-hydro- | | U.S. Pat. No. 5134155 |
| indazolyl, | | |
| tetrahydrocyclo- | | |
| pentapyrazolyl, or hexa- | | |
| hydrocyclohepta- | | |
| pyrazole)-heptenoate | | |
| derivatives | | |
| HMG Co-A Reductase | | British Biotech & Japan |
| Inhibitors | | Tobacco |
| HMG Co-A Reductase | | Merck & Co. |
| Inhibitors | | |
| A-1233 | | Kitasato University |
| BAY-w-9533 | | Bayer |
| BB-476 | | British Biotech |
| BMS-180436 | | Bristol-Myers Squibb |
| BMY-22566 | | |
| HMG Co-A Reductase | | Bristol-Myers Squibb |
| Inhibitors | | |
| HMG Co-A Reductase | | Ono |
| Inhibitors | | |
| HMG Co-A Reductase | | Chiroscience |
| Inhibitors, Chiral | | |
| HMG Co-A Reductase | | Nissan Chemical |
| Inhibitors, isoxazolo- | | |
| pyridine | | |
| HMG Co-A Reductase | | Pharmacia & Upjohn |
| Inhibitors, seco-oxysterol | | |
| HMG Co-A Reductase | | Sandoz |
| Inhibitors, thiophene | | |
| HMG Co-A Reductase | | Hoechest Marion Roussel |
| Inhibitors, 6-phenoxy- | | |
| 3,5-dihydroxyhexanoic | | |
| acids | | |
| Hypolipaemics | | Warner-Lambert |
| N-((1-methylpropyl)- | | Sandoz |
| carbonyl)-8-(2- | | |
| (tetrahydro-4-hydroxy-6- | | |
| oxo-2H-pyran-2- | | |
| yl)ethyl)-perhydro- | | |
| isoquinoline | | |
| N-(1-oxododecyl)-4α,10- | | Hoechst Marion Roussel |
| dimethyl-8-aza-trans- | | |
| decal-3β-ol | | |
| P-882222 | | Nissan Chemical |
| S-853758A | | Hoechst Marion Roussel |
| (S)-4-((2-(4-(4- | | Bristol-Myers Squibb |
| fluorophenyl)-5-methyl- | | |
| 2-(1-methylethyl)-6- | | |
| phenyl-3-pyridinyl)- | | |
| ethenyl)hydroxy- | | |
| phosphinyl)-3- | | |
| hydroxybutanoic acid, | | |
| disodium salt | | |
| SDZ-265859 | | Sandoz |
| (4R-(4α,6β(E)))-6-(2-(5- | | Warner Lambert |
| (4-fluorophenyl)-3-(1- | | |
| methyl-ethyl)-1-(2- | | |
| pyridinyH-pyrazol-4- | | |
| yl)ethenyl)tetra-hydro-4- | | |
| hydroxy-2H-pyran-2-one | | |
| 5β-aminoethyl- | | Boehringer Mannheim |
| thiopentanoic acid | | |
| derivatives | | |
| 6-amino-2-mercapto-5- | | North Carolina |
| methylpyrimidine-4- | | University |
| carboxylic acid | | |
| 6-phenoxymethyl -and 6- | | Hoechst Marion Roussel |
| phenylethylen-(4- | | |
| hydroxy-tetrahydropyran- | | |
| 2-one) analogues | | |

In one embodiment, the statin is selected from the group consisting of mevastatin, lovastatin, simvastatin, pravastatin, fluvastatin, atorvastatin, cerivastatin, bervastatin, ZD-4522, BMS 180431, NK-104, carvastatin, PD-135022, crilvastatin, acitemate, DMP-565, glenvastatin, L-659699, L-669262, S-2467, and S-2468.

In another embodiment, the statin is selected from the statins listed in Table 4 below. The individual patent documents referenced in Table 4 describe the preparation of these statins and are each herein incorporated by reference.

TABLE 4

| | | CAS | Patent/Literature Reference |
|---|---|---|---|
| Compound | Common | Registry | for Preparation of |
| Number | Name | Number | Compound Per Se |
| B-1 | Mevastatin | 73573-88-3 | U.S. Pat. No. 3,983,140 |
| B-2 | Lovastatin | 75330-75-5 | U.S. Pat. No. 4,231,938 |
| B-3 | Simvastatin | 79902-63-9 | U.S. Pat. No. 4,444,784 |
| B-4 | Pravastatin | 81093-37-0 | U.S. Pat. No. 4,346,227 |
| B-5 | Fluvastatin | 93957-54-1 | U.S. Pat. No. 4,739,073; |
| | | | U.S. Pat. No. 5,354,772 |
| B-6 | Atorvastatin | 134523-00-5 | EP 409281; |
| | | | U.S. Pat. No. 5,273,995 |
| B-7 | Cerivastatin | 145599-86-6 | U.S. Pat. No. 5,177,080 |
| B-8 | ZD-4522 | 147098-20-2 | EP 521471, Example 7; |
| | | | Bioorg. Med. Chem., Vol. |
| | | | 5(2), pp. 437-444 (1997); |
| | | | Drugs Future, Vol. 24 (5), pp. |
| | | | 511-513 (1999) |
| B-9 | NK-104 | 141750-63-2 | EP 0304063; |
| | | | CA 1336714 |

In another embodiment, the statin is selected from the group of statins consisting of lovastatin, simvastatin, pravastatin, atorvastatin, cerivastatin, ZD-4522 and NK-104.

In another embodiment, the statin is selected from the group of statins consisting of lovastatin, simvastatin, pravastatin, atorvastatin, and ZD-4522.

In another embodiment, the statin is selected from the group of statins consisting of simvastatin, pravastatin, atorvastatin, and ZD-4522.

In another embodiment, the statin is selected from the group of statins consisting of cerivastatin, ZD-4522 and NK-104.

In another embodiment, the statin is selected from the group of statins consisting of ZD-4522 and NK-104.

In another embodiment, the statin is selected from the group of statins consisting of lovastatin, simvastatin, pravastatin, and atorvastatin.

As noted above, the ASBT inhibitors and MG Co-A reductase inhibitors useful in the present combination therapy also may include the racemates and stereoisomers, such as diastereomers and enantiomers, of such inhibitors. Such stereoisomers can be prepared and separated using conventional techniques, either by reacting enantiomeric starting materials, or by separating isomers of compounds of the present invention. Isomers may include geometric isomers, for example cis isomers or trans isomers across a double bond. All such isomers are contemplated among the compounds of the present invention. Such isomers may be used in either pure form or in admixture with those inhibitors described above.

In addition to being particularly suitable for human use, the present combination therapy is also suitable for treatment of animals, including mammals such as horses, dogs, cats, rats, mice, sheep, pigs, and the like.

Definitions

The term “subject” as used herein refers to an animal, preferably a mammal, and particularly a human, who has been the object of treatment, observation or experiment.

The term “treatment” refers to any process, action, application, therapy, or the like, wherein a subject, including a human being, is provided medical aid with the object of improving the subject's condition, directly or indirectly, or slowing the progression of a condition or disorder in the subject.

The terms “prophylaxis” and “prevention” include either preventing the onset of a clinically evident condition or disorder altogether or preventing the onset of a preclinically evident stage of a condition or disorder in a subject. These terms encompass, but are not limited to, the prophylactic treatment of a subject at risk of developing a hyperlipidemic condition or disorder such as, but not limited to, atherosclerosis, and hypercholesterolemia.

The term “combination therapy” means the administration of two or more therapeutic agents to treat a condition and/or disorder in a subject, for example, the treatment of a hyperlipidemic condition or disorder such as atherosclerosis or hypercholesterolemia. Such administration encompasses co-administration of these therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner. In either case, the treatment regimen will provide beneficial effects of the drug combination in treating the condition.

The phrase “therapeutically-effective” qualifies the amount of each agent that will achieve the goal of improvement in condition or disorder severity and the frequency of incidence over treatment of each agent by itself, while avoiding adverse side effects typically associated with alternative therapies.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

Mechanism of Action

Without being held to a specific mechanism of action for the present combination therapy, it is hypothesized that the administration of these selected ASBT inhibitors and HMG Co-A reductase inhibitors in combination is effective because of the simultaneous and interrelated responses of the liver to these two distinct classes of drugs: marked upregulation of bile acid synthesis in response to the ASBT inhibitor (consuming cholesterol to form bile acids) and potent inhibition of de novo cholesterol synthesis in the liver in response to the HMG Co-A reductase inhibitor. As a result of the combination treatment with these two drugs working by these two different mechanisms, upregulation of LDL receptor expression on the surface of hepatocytes is effectively the only way to provide the liver with cholesterol essential to support bile acid synthesis and maintain the total bile acid pool. Accordingly, serum cholesterol levels are lowered as serum cholesterol is taken up by the liver and consumed in the synthesis of bile acids.

Advantages of Combination Therapy

The selected ASBT inhibitors and HMG Co-A reductase inhibitors of the present invention act in combination to provide more than an additive benefit. The cholesterol-lowering effect of the combination therapy methods described herein is greater than the the cholesterol-lowering effect seen with the monotherapeutic administration of each active agent alone, as well as the sum of the cholesterol-lowering effects achieved by administering the ASBT inhibitor and HMG Co-A reductase inhibitor separately in monotherapeutic treatment. The present invention, for example, provides greater dosing flexibility and/or permits a reduction in the dosages of ASBT inhibitor and/or HMG Co-A reductase inhibitor administered to a subject relative to the corresponding monotherapeutic dosages without adversely affecting the efficacy of the therapy. Elevating bile acid excretion even a small amount with the ASBT inhibitor will increase bile acid synthesis to restore the total body pool of bile acids. This synthesis consumes liver cholesterol as a metabolic precursor to bile acids. Blocking the synthesis of liver cholesterol with the HMG Co-A reductase inhibitors will enhance upregulation of expression of the LDL receptor thereby increasing uptake of serum LDL cholesterol. The amounts of the drugs required to obtain a comparable reduction in serum total cholesterol are materially lower than the “additive line” for the two drugs (i.e., well below the line representing minus one standard deviation for the additive effect). This finding indicates that the combination treatment produces an effect beyond a mere additive effect for the two drugs.

The methods of this invention also provide for the effective prophylaxis and/or treatment of hyperlipidemic conditions and/or disorders with reduced side effects compared to conventional methods known in the art. For example, administration of HMG Co-A reductase inhibitors can result in side effects such as, but not limited to, rhabdomyocytis, elevated liver enzymes, constipation, abdominal pain, dyspepsia, diarrhea, fever, flatulence, headache, myopathy, sinusitus, pharyngitis, myalgia, arthralgia, asthenia, and backpain. Rhabdomyocitis (muscle pain) and elevated liver enzymes (e.g., transaminases) occur more frequently at the highest recommended doses of most HMG Co-A reductase inhibitors. Reduction of the HMG Co-A reductase inhibitor doses in the present combination therapy below conventional monotherapeutic doses will minimize, or even eliminate, the side-effect profile associated with the present combination therapy relative to the side-effect profiles associated with, for example, monotherapeutic administration of HMG Co-A reductase inhibitors.

Periodic liver enzyme testing, typically every six months, is a routine procedure for subjects undergoing monotherapy with HMG Co-A reductase inhibitors. Because the present combination therapy minimizes or eliminates the presence of elevated liver enzymes, liver enzyme testing of subjects undergoing the present combination therapy may be discontinued or required at a much lower frequency than for HMG Co-A reductase inhibitor monotherapy. The side effects associated with the HMG Co-A reductase inhibitors typically are dose-dependent and, thus, their incidence increases at higher doses. Accordingly, lower effective doses of the HMG Co-A reductase inhibitors will result in fewer side effects than seen with higher doses of HMG Co-A reductase inhibitors in monotherapy or decrease the severity of such side-effects.

Other benefits of the present combination therapy include, but are not limited to, the use of a selected group of ASBT inhibitors that provide a relatively quick onset of therapeutic effect and a relatively long duration of action. For example, a single dose of one of the selected ASBT inhibitors may stay associated with the transporter in a manner that can affect multiple cycles of bile acid recirculation.

Dosages and Treatment Regimen

Dosage levels of the selected ASBT inhibitors useful in the present combination therapy typically are on the order of about 0.001 mg to about 10,000 mg daily, with preferred levels of about 0.005 mg to about 1,000 mg daily, more preferred levels of about 0.008 to about 100 mg daily, and still more preferred levels of about 0.01 mg to about 40 mg daily.

Dosage levels of the selected HMG Co-A reductase inhibitors useful in the present combination therapy typically are on the order of about 0.001 mg to about 1,000 mg daily, with preferred levels of about 0.01 mg to about 500 mg daily, and more preferred levels of about 0.05 to about 100 mg daily. The preferred daily dosage of each HMG Co-A reductase inhibitor selected typically will be lower than the dosage recommended for conventional monotherapeutic treatment with that HMG Co-A reductase inhibitor. Examples of such conventionally recommended monotherapeutic dosages include about 10 to 80 mg for atorvastatin (for example, LIPITOR®); about 5 to 80 mg for simvastatin (for example, ZOCOR®); about 10 to 40 mg for pravastatin (for example, PRAVACHOL®); about 20 to 80 mg for lovastatin (for example, MEVACOR®); about 0.2 to 0.4 mg for cerivastatin (for example, BAYCOL®); and about 20 to 80 mg for fluvastatin (for example, LESCOL®).

It is understood, however, that the specific dose level for each patient will depend upon a variety of factors including the activity of the specific inhibitors employed, the age, body weight, general health, sex, diet, time of administration, rate of excretion, inhibitor combination selected, the severity of the particular conditions or disorder being treated, and the form of administration. Appropriate dosages can be determined in trials. The ratio of ASBT inhibitor to HMG Co-A reductase inhibitor (weight/weight), however, typically will range from about 1:100 to about 100:1, preferably about 1:50 to about 3:1, more preferably about 1:20 to about 2:1, and still more preferably about 1:20 to about 1.5:1.

The total daily dose of each drug can be administered to the patient in a single dose, or in proportionate multiple subdoses. Subdoses can be administered two to six times per day. Doses can be in immediate release form or sustained release form effective to obtain desired results. Single dosage forms comprising the ASBT inhibitor and the HMG Co-A reductase inhibitor may be used where desirable.

Crystalline Forms of Active Compounds

It is particularly useful to select a form of each active compound that is easily handled, reproducible in form, easily prepared, and which is non-hygroscopic. A hygroscopic compound can absorb water, for example, from the ambient atmosphere, and a sample of the compound can gain weight as more water is absorbed. Absorbance of water into a sample of a compound can also affect measurements of the compound, for example, infrared spectra. Hygroscopicity of a pharmaceutical compound can be problematic if that compound absorbs water to an extent and at such a rate that weighing and measurement of the compound is made difficult. Accurate weighing and measurement of a pharmaceutical compound is important to assure that patients receive an appropriate dose. By way of illustration and not limitation, several crystalline forms have been identified for Compound A-5, particularly the (4R.5R) configuration of Compound A-5 disclosed as compound 41 of Example 29 below.

A first crystalline form (Form I) of compound 41 or its enantiomer has a melting point or a decomposition point of about 220° C. to about 235° C., generally about 228° C. to about 232° C., and more typically about 230° C. Form I can be prepared, for example, by crystallization of compound 41 or its enantiomer from a solvent which comprises acetonitrile, methanol, or methyl t-butyl ether. Preferably, Form I can be prepared by crystallization of compound 41 or its enantiomer from a solvent comprising methanol or methyl t-butyl ether, and more preferably from a solvent comprising methanol and methyl t-butyl ether. Methods for the preparation of Form I include those described in Examples 1426 and 1426a of U.S. Pat. No. 5,994,391, which patent is herein incorporated by reference.

A second crystalline form (Form II) of compound 41 or its enantiomer has a melting point or a decomposition point of about 278° C. to about 285° C. Form II generally has a melting point or a decomposition point of about 280° C. to about 283° C., and more typically about 282° C. The (4R,5R) configuration is a preferred absolute configuration for the compound forming the crystal structure of Form II. The enantiomer having a (4S,5S) absolute configuration, however, can also be prepared in the crystalline form of the present invention.

Form II can be prepared, for example, by crystallization of compound 41 or its enantiomer from a solvent, preferably a ketone solvent, more preferably a ketone solvent comprising methyl ethyl ketone (MEK) or acetone. By way of example, compound 41 or its (4S,5S) enantiomer can be mixed in a solvent comprising MEK and Form II can be induced to crystallize from that solution. Preferably, compound 41 or its (4S,5S) enantiomer is dissolved in a solvent comprising a ketone such as MEK and a quantity of water (for example about 0.5% to about 5% water by weight, preferably 1% to about 4% water by weight, and more preferably 2% to about 4% water by weight). The crystallization can be induced, for example, by evaporating the solvent (e.g., by distillation or by exposure to a stream of a gas such as air or nitrogen for a period of time) or by evaporating the water (e.g. by distillation or azeotroping). Alternatively, the crystallization will be induced by other traditional crystallization methods such as chilling or by addition of another solvent or by addition of a seed crystal. As another alternative, crystallization can be induced by adding more MEK (decreasing the percent by weight of water in the crystallization solvent). Form II can conveniently be caused to precipitate from a reaction mixture in which compound 41 is prepared (e.g., the reaction of (4R,5R)-27 with DABCO as disclosed in the working examples below) by running that reaction in a solvent comprising MEK, and preferably in a solvent comprising MEK and about 0.5% to about 5% by weight of water. The precipitation can be facilitated by distilling solvent off of the reaction mixture.

FIG. 1 shows typical X-ray powder diffraction patterns for Form I (plot (a)) and Form II (plot (b)) of compound 41. The Form II crystalline form generally has the X-ray powder diffraction pattern shown in FIG. 1, plot (b). Typically, Form II has an X-ray powder diffraction pattern with peaks at about 9.2 degrees 2 q, about 12.3 degrees 2 q, and about 13.9 degrees 2 q. The Form II X-ray powder diffraction pattern typically lacks peaks at about 7.2 degrees 2 q and at about 11.2 degrees 2 q. Table X-130 in Example 130 below shows a comparison of prominent X-ray powder diffraction peaks for Form I and Form II.

FIG. 2 shows typical Fourier transform infrared (“IR”) spectra for Form I (plot (a)) and Form II (plot (b)) of compound 41. The Form II crystalline form generally has the IR spectrum shown in FIG. 2, plot (b). Typically, Form II has an IR spectrum with a peak at about 3245 cm⁻¹ to about 3255 cm⁻¹. Form II typically also has an IR peak at about 1600 cm⁻¹. Form II typcially also has another IR peak at about 1288 cm⁻¹. Table X-131 in Example 131 below shows a comparison of prominent FTIR peaks for Form I and Form II.

FIG. 3 shows typical solid state carbon-13 nuclear magnetic resonance (“NMR”) spectra for Form I (plot (a)) and Form II (plot (b)) of compound 41. The Form II crystalline form generally has the solid state carbon-13 NMR spectrum shown in FIG. 3, plot (b). Typically, Form II has a solid state carbon-13 NMR spectrum with peaks at about 142.3 ppm, about 137.2 ppm, and about 125.4 ppm. Table X-132 in Example 132 below shows a comparison of prominent solid state carbon-13 NMR peaks for Form I and Form II.

FIG. 4 shows typical differential scanning calorimetry profiles for Form I (plot (a)) and Form II (plot (b)) of compound 41.

A dry sample of the crystalline form having a melting point or a decomposition point of about 278° C. to about 285° C. (i.e., Form II) typically gains less than about 1% of its own weight when equilibrated under 80% relative humidity (RH) air at 25° C. Such a crystalline form is essentially non-hygroscopic. For example, when a sample of Form II crystalline form of compound 41 or an enantiomer thereof is dried at essentially 0% RH at about 25° C. under a purge of essentially dry nitrogen until the sample exhibits essentially no weight change as a function of time, the sample gains less than 1% of its own weight when it is then equilibrated under about 80% RH air at about 25° C. For the present purposes, the term “essentially 0% RH” means less than about 1% RH. The term “equilibrated” means that the change in weight of a sample over time at a given relative humidity is less than 0.0003% ((dm/dt)/m₀×100, where m is mass in mg, m₀ is initial mass, and t is time in minutes).

Therefore, in one embodiment the ASBT inhibitor selected is a crystalline form (i.e., Form II) of Compound A-5 having a melting point or a decomposition point of about 278° C. to about 285° C. Form II generally has a melting point or a decomposition point of about 280° C. to about 283° C., and more typically about 282° C. Preferably, Compound A-5 has an absolute configuration of (4R,5R) (i.e., compound 41) and this is a preferred absolute configuration for the compound forming the crystal structure of Form II. However, the (4S,5S) enantiomer of Compound A-5 can also be prepared in the crystalline form of the present invention.

In another embodiment, the ASBT inhibitor selected is a crystalline form (i.e., Form I) of Compound A-5 having a melting point or a decomposition point of about 220° C. to about 235° C. Form I generally has a melting point or a decomposition point of about 228° C. to about 232° C., and more typically about 230° C. Preferably, Compound A-5 has an absolute configuration of (4R,5R) (i.e., compound 41) and this is a preferred absolute configuration for the compound forming the crystal structure of Form I. However, the (4S,5S) enantiomer of Compound A-5 can also be prepared in the crystalline form of the present invention.

In yet another embodiment, the ASBT inhibitor selected is a crystalline form of Compound A-5 having an absolute configuration of (4R,5R) and a melting point or a decomposition point of about 278° C. to about 285° C. (i.e., Form II), and the HMG Co-A reductase inhibitor is selected from the group consisting of atorvastatin, simvastatin, pravastatin, lovastatin, and ZD-4522.

Combinations and Compositions

The present invention is further directed to combinations, including pharmaceutical compositions, comprising one or more ASBT inhibitors selected from the group consisting of compounds A-1 through A-15 described above, and one or more HMG Co-A reductase inhibitors. In one embodiment, the present invention comprises a first amount of the ASBT inhibitor, or a pharmaceutically acceptable salt, ester, or prodrug thereof; a second amount of the HMG Co-A reductase inhibitor, or a pharmaceutically acceptable salt, ester, conjugate acid, or prodrug thereof; and a pharmaceutically acceptable carrier. Preferably, the first and second amounts of the inhibitors together comprise a therapeutically effective amount of the inhibitors. The preferred ASBT inhibitors and HMG Co-A reductase inhibitors used in the preparation of the compositions are as previously set forth above. The combinations and compositions comprising an ASBT inhibitor and an HMG Co-A reductase inhibitor of the present invention can be administered for the prophylaxis and/or treatment of hyperlipidermic conditions and/or disorders by any means that produce contact of these inhibitors with their site of action in the body, for example in the ileum of a human for the ASBT inhibitor.

For the prophylaxis or treatment of the conditions and disorders referred to above, the combination administered can comprise the inhibitor compounds per se. Alternatively, pharmaceutically acceptable salts are particularly suitable for medical applications because of their greater aqueous solubility relative to the parent compound.

The combinations of the present invention also can be presented with an acceptable carrier in the form of a pharmaceutical composition. The carrier must be acceptable in the sense of being compatible with the other ingredients of the composition and must not be deleterious to the recipient. The carrier can be a solid or a liquid, or both, and preferably is formulated with the compound as a unit-dose composition, for example, a tablet, which can contain from 0.05% to 95% by weight of the active compounds. Other pharmacologically active substances can also be present, including other compounds useful in the present invention. The pharmaceutical compositions of the invention can be prepared by any of the well-known techniques of pharmacy, such as admixing the components.

The combinations and compositions of the present invention can be administered by any conventional means available for use in conjunction with pharmaceuticals, either as the ASBT inhibitor and HMG Co-A reductase inhibitor combination alone or in further combination with other therapeutic compounds. Oral delivery of the ASBT inhibitor and the HMG Co-A reductase inhibitor is generally preferred (although the methods of the present invention are still effective, for example, if the HMG Co-A reductase inhibitor is administered parenterally). The amount of each inhibitor in the combination or composition that is required to achieve the desired biological effect will depend on a number of factors including those discussed below with respect to the treatment regimen.

Orally administrable unit dose formulations, such as tablets or capsules, can contain, for example, from about 0.01 to about 500 mg, preferably about 0.05 mg to about 100 mg, and more preferably from about 0.1 to about 50 mg, of the ASBT inhibitor, and/or from about 0.01 to about 500 mg, preferably about 0.05 mg to about 100 mg, and more preferably from about 0.1 to about 50 mg, of the HMG Co-A reductase inhibitor. In the case of pharmaceutically acceptable salts, the weights indicated above for the ASBT inhibitors refer to the weight of the pharmaceutically active ion derived from the salt.

Oral delivery of the ASBT inhibitors and the HMG Co-A reductase inhibitors of the present invention can include formulations, as are well known in the art, to provide immediate delivery or prolonged or sustained delivery of the drug to the gastrointestinal tract by any number of mechanisms. Immediate delivery formulations include, but are not limited to, oral solutions, oral suspensions, fast-dissolving tablets or capsules, disintegrating tablets and the like. Prolonged or sustained delivery formulations include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action (for example, the ileum for the ASBT inhibitor) by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester. Such prolonged or sustained delivery formulations preferably are in dispersed form at the time they reach the ileum.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the inhibitor(s) and the carrier (which can constitute one or more accessory ingredients). In general, the compositions are prepared by uniformly and intimately admixing the inhibitor(s) with a liquid or finely divided solid carrier, or both, and then, if necessary, shaping the product. For example, a tablet can be prepared by compressing or molding a powder or granules of the inhibitors, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the inhibitors in an inert base such as gelatin and glycerin or sucrose and acacia.

In any case, the amount of ASBT inhibitor and HMG Co-A reductase inhibitor that can be combined with carrier materials to produce a single dosage form to be administered will vary depending upon the host treated and the particular mode of administration. The solid dosage forms for oral administration including capsules, tablets, pills, powders, and granules noted above comprise the inhibitors of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Pharmaceutically acceptable carriers encompass all the foregoing and the like. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

Dosage Regimen

As noted above, the dosage regimen to prevent, treat, give relief from, or ameliorate a hyperlipidemic condition or disorder, or to otherwise protect against or treat further high cholesterol plasma or blood levels with the combinations and compositions of the present invention is selected in accordance with a variety of factors. These factors include the type, age, weight, sex, diet, and medical condition of the patient, the severity of the disease, the route of administration, pharmacological considerations such as the activity, efficacy, pharmacokinetics and toxicology profiles of the particular inhibitors employed, whether a drug delivery system is utilized, and whether the inhibitors are administered with other active ingredients. Thus, the dosage regimen actually employed may vary widely and therefore deviate from the preferred dosage regimen set forth above.

Initial treatment of a patient suffering from a hyperlipidemic condition or disorder can begin with the dosages indicated above. Treatment generally should be continued as necessary over a period of several weeks to several months or years until the hyperlipidemic condition or disorder has been controlled or eliminated. Patients undergoing treatment with the combinations or compositions disclosed herein can be routinely monitored, for example, by measuring serum LDL and total cholesterol levels by any of the methods well-known in the art, to determine the effectiveness of the combination therapy. Continuous analysis of such data permits modification of the treatment regimen during therapy so that optimal effective amounts of each type of inhibitor are administered at any time, and so that the duration of treatment can be determined as well. In this way, the treatment regimen/dosing schedule can be rationally modified over the course of therapy so that the lowest amount of ASBT inhibitor and HMG Co-A reductase inhibitor that together exhibit satisfactory effectiveness is administered, and so that administration is continued only so long as is necessary to successfully treat the hyperlipidemic condition.

In combination therapy, administration of the ASBT inhibitor and the HMG Co-A reductase inhibitor may take place sequentially in separate formulations, or may be accomplished by simultaneous administration in a single formulation or separate formulations. Administration may be accomplished by any appropriate route, with oral administration being preferred. The dosage units used may with advantage contain one or more ASBT inhibitors and one or more HMG Co-A reductase inhibitors in the amounts described above.

Dosing for oral administration may be with a regimen calling for a single daily dose, for multiple, spaced doses throughout the day, for a single dose every other day, for a single dose every several days, or other appropriate regimens. The ASBT inhibitors and the HMG Co-A reductase inhibitor used in the combination therapy may be administered simultaneously, either in a combined dosage form or in separate dosage forms intended for substantially simultaneous oral administration. The ASBT inhibitors and the HMG Co-A reductase inhibitors also may be administered sequentially, with either inhibitor being administered by a regimen calling for two-step ingestion. Thus, a regimen may call for sequential administration of the ASBT inhibitor and the HMG Co-A reductase inhibitor with spaced-apart ingestion of these separate, active agents. The time period between the multiple ingestion steps may range from a few minutes to several hours, depending upon the properties of each active agent such as potency, solubility, bioavailability, plasma half-life and kinetic profile of the inhibitor, as well as depending upon the age and condition of the patient. The combination therapy, whether administration is simultaneous, substantially simultaneous, or sequential, may involve a regimen calling for administration of the ASBT inhibitor by oral route and the HMG CoA reductase inhibitor by intravenous route. Whether these active agents are administered by oral or intravenous route, separately or together, each such active agent will be contained in a suitable pharmaceutical formulation of pharmaceutically acceptable excipients, diluents or other formulations components. Examples of suitable pharmaceutically acceptable formulations are given above.

Kits

The present invention further comprises kits that are suitable for use in performing the methods of treatment and/or prophylaxis described above. In one embodiment, the kit contains a first dosage form comprising one or more of the ASBT inhibitors identified in Table 1 and a second dosage form comprising an HMG Co-A reductase inhibitor identified in Table 4 in quantities sufficient to carry out the methods of the present invention. Preferably, the first dosage form and the second dosage form together comprise a therapeutically effective amount of the inhibitors for the prophylaxis and/or treatment of a hyperlipidemic condition and/or disorder.

The methods, combinations, compositions and kits of the present invention also are useful for the prophylaxis and/or treatment of gallstones.

The methods, combinations, compositions and kits of the present invention also are useful for the prophylaxis and treatment of conditions related to bone formation and resorption.

The following nonlimiting examples serve to illustrate the various aspects of the present invention.

### EXAMPLE 1

### Monotherapeutic Treatment with Compound A-5

Male beagle dogs (9-10 kg) obtained from Marshall farms were fed a normal chow diet once a day for a two hour interval and given water ad libitum. Prior to initiating treatment, the dogs were weighed and blood samples were drawn from the cephalic vein of each dog following an overnight fast to evaluate pretreatment serum total cholesterol levels at the start of the study. The dogs were randomly assigned to one of the five treatment groups (n=6 per group) such that each group had mean serum total cholesterol values and body weights within 5% of each other. Each treatment group received one of the following dosages: (1) vehicle (containing no compound A-5), (2) 0.22 mg/kg/day of compound A-5, (3) 0.66 mg/kg/day of compound A-5, (4) 2.0 mg/kg/day of compound A-5, and (5) 6.0 mg/kg/day of compound A-5. All doses were administered in gelatin capsules per os to each dog between 9:00-9:30 a.m. prior to feeding. All animals were fed between 9:30-10:00 a.m. and were allowed two hours to eat, at which time any remaining food was removed. Typically, most dogs had consumed their entire meal within this time period. Animals were dosed daily for three weeks and blood samples were taken at the end of each week after an overnight fast for comparison with pretreatment serum total cholesterol levels. Three consecutive 24 hour fecal samples were collected for each group during the last 72 hours of each week and used to measure the concentration of fecal biles acids excreted during that time period.

Serum Lipid Measurements

Blood was collected from the cephalic vein of each dog into serum separator tubes. The blood was centrifuged at 900×g for 20 minutes at room temperature and the serum decanted. All analyses were performed on a Cobas Mira Clinical Analyzer System (Roche Diagnostic Systems, Branchburg, N.J.) using Roche Diagnostic reagents for enzymatic determinations of serum cholesterol. Commercial calibrator and quality control materials were analyzed with each run to verify assay accuracy and precision. A two-tailed, paired Students t-test was used to determine the statistical significance of changes in serum total cholesterol in treated dogs compared to pretreatment values. A one-way analysis of variance (“ANOVA”) was used to compare each pair of treatment groups to determine the statistical significance of changes in serum total cholesterol.

Table X-1A below reports the data measured on the effect of compound A-5 monotherapy at four different dosages on serum total cholesterol.

TABLE X-1A

| | SERUM TOTAL CHOLESTEROL | | | |
|---|---|---|---|---|
| COMPOUND | (mg/dL) | | | |
| A-5 DOSAGE | Pretreatment | Week 1 | Week 2 | Week 3 |
| Vehicle | 155 ± 11¹ | 152 ± 12 | 153 ± 15 | 149 ± 14 |
| | | (−2)² | (−1) | (−4) |
| 0.22 mg/kg/day | 154 ± 9 | 140 ± 10³ | 143 ± 11 | 138 ± 11³ |
| | | (−9) | (−7) | (−10) |
| 0.66 mg/kg/day | 156 ± 11 | 145 ± 9 | 149 ± 8 | 146 ± 8 |
| | | (−7) | (−4) | (−6) |
| 2.0 mg/kg/day | 156 ± 11 | 137 ± 10³ | 133 ± 11³ | 136 ± 10³ |
| | | (−12) | (−15) | (−13) |
| 6.0 mg/kg/day | 158 ± 11 | 128 ± 11³ | 124 ± 11³ | 120 ± 11³ |
| | | (−19) | (−22) | (−24) |

¹All values shown are mean ± SEM, n = 6.
²( ) = % change in serum total cholesterol compared to the pretreatment value of each group.
³P < 0.05 versus the pretreatment serum total cholesterol value for each group by a paired, two-tailed t-test without equal variance assumption.

Fecal Bile Acid Measurement

Fecal samples were collected to determine the fecal bile acid (“FBA”) concentration for each animal. Three consecutive 24 hour fecal samples were collected between 8:00 a.m. and 9:00 a.m. each day, prior to dosing and feeding, during the last 72 hour period of each week. The separate daily collections from each dog were weighed, combined and homogenized with distilled water in a food processor to generate a homogeneous slurry. A 1.4 g sample of fecal homogenate was extracted with 2.6 mL of a solution containing tertiary butanol:distilled water in the ratio of 2:0.6 (final concentration of 50% tertiary butanol v/v distilled water) for 45 minutes in a 37° C. water bath and centrifuged for 13 minutes at 2000×g. The concentration of bile acids (μmoles/gram homogenate) was determined using a 96-well enzymatic assay system described in van der Meer et al., “t-Butanol Extraction of Feces: A Rapid Procedure For Enzymic Determination Of Fecal Bile Acids”, Cholesterol, Metabolism in Health and Disease: Studies in the Netherlands, edited by Beynen, et al., Ponsen and Looyen, Wageningen, 1985; and Turley et al., “Re-evaluation of the 3 Alpha-Hydroxysteroid Dehydrogenase Assay For Total Bile Acids In Bile”, J. Lipid Research, 19:924-928, 1978.

A 20 μl aliquot of each fecal extract was added to each of two sets of triplicate wells in a 96-well assay plate. A standardized sodium taurocholate solution and a standardized fecal extract solution (previously made from pooled samples and characterized for its bile acid concentration) were also analyzed for assay quality control. A standard curve of five points containing 30-540 nmoles/well was generated by serial dilutions of an initial 20 μL aliquot of 90 mM sodium taurocholate. A 230 μL aliquot of a reaction mixture containing 1M hydrazine hydrate, 0.1 M pyrophosphate and 0.46 mg/ml NAD was added to each well. Subsequently, a 50 μL aliquot of either 3α-hydroxysteroid dehydrogenase (“HSD”) enzyme (0.8 units/mL) or assay buffer (0.1 M sodium pyrophosphate) was then added to one each of the two sets of triplicates. Following 60 minutes of incubation at room temperature, the optical density at 340 nm was measured and the mean of each set of triplicate samples was calculated. The difference in optical density with and without HSD enzyme was used to determine the bile acid concentration (mM) of each sample based on the sodium taurocholate standard curve. The bile acid concentration of the extract (μmoles/gram homogenate), the total weight of the fecal homogenate (grams) and the body weight of the dogs (kg) were used to calculate the corresponding fecal bile acid concentration in μmoles/kg/day for each animal.

All reagents used for the assay were obtained from Sigma Chemical Co., St. Louis, Mo. (HSD enzyme—catalog # H-1506; NAD—catalog # N1636; sodium taurocholate—catalog # T4009). A one-tailed, two-sample Students t-Test without assumption of equal variance was used to determine the statistical significance of changes in fecal bile acid concentration in treated animals compared to vehicle animals and between treatment groups.

Table X-1B below reports the data measured on the effect of the compound A-5 monotherapy therapy on fecal bile acid concentration.

TABLE X-1B

| | FECAL BILE ACID CONCENTRATION | | |
|---|---|---|---|
| COMPOUND A-5 | (μmol/day/kg) | | |
| DOSAGE | Week 1 | Week 2 | Week 3 |
| Vehicle | 27 ± 3¹ | 27 ± 7 | 29 ± 4 |
| 0.22 mg/kg/day | 89 ± 9³ | 100 ± 11³ | 81 ± 7³ |
| | (230)² | (270) | (179) |
| 0.66 mg/kg/day | 134 ± 19³ | 134 ± 16³ | 126 ± 12³ |
| | (396) | (396) | (334) |
| 2.0 mg/kg/day | 168 ± 13³ | 138 ± 12³ | 164 ± 11³ |
| | (522) | (411) | (465) |
| 6.0 mg/kg/day | 190 ± 18³ | 209 ± 27³ | 179 ± 16³ |
| | (604) | (674) | (517) |

¹All values shown are mean ± SEM, n = 6.
²( ) = % change in fecal bile acid compared to the pretreatment value of each group.
³P < 0.01 versus fecal bile acid concentration of the vehicle group at each time point, by a two-sample, one-tailed t-test without equal variance assumption.

Results

There were no significant changes in body or fecal weights, stool consistency or general animal health for any of the groups throughout this study. Treatment with compound A-5 stimulated a dose-related increase in the concentration of fecal bile acid that was statistically significant (P<0.01) compared to the vehicle group at all doses and time points. The maximal effect of compound A-5 on increasing fecal bile acid excretion was observed to occur within the first week of treatment and was maintained throughout the following two week period of the study. Fecal bile acid concentration was increased by 230%, 396%, 522% and 604% following one week of treatment and by 179%, 334%, 465% and 517% following three weeks of treatment compared to vehicle at 0.22, 0.66, 2.0 and 6.0 mg/kg/day doses of compound A-5, respectively.

Compound A-5 also stimulated a dose-related decrease in serum total cholesterol that was statistically significant (p<0.05) compared to the vehicle group at all three time points for the 2.0 and 6.0 mg/kg/day doses. Although reductions in serum total cholesterol ranged from 4% to 10% for the two lower doses of compound A-5, only those for 0.22 mg/kg/day at one and three weeks were determined to be statistically significant. The majority of the effect of compound A-5 on reducing serum total cholesterol was observed to occur within the first week of treatment and was maintained at approximately the same level throughout the following two week period of the study. Serum total cholesterol concentration was decreased by 9%, 7%, 12% and 19% following one week of treatment and by 10%, 6%, 13% and 24% following three weeks of treatment compared to vehicle at 0.22, 0.66, 2.0 and 6.0 mg/kg/day doses of compound A-5, respectively.

### EXAMPLE 2

### Monotherapeutic Treatment with Pravastatin

Beagle dogs also were administered pravastatin to evaluate the monotherapeutic effect of pravastatin on serum total cholesterol. The protocol described in Example 1 for determination of serum total cholesterol in dogs undergoing compound A-5 monotherapy was generally followed. Instead of receiving compound A-5, however, the dogs received one of the following daily dosages of pravastatin: (1) vehicle (containing no pravastatin), (2) 0.25 mg/kg/day pravastatin, (3) 1.0 mg/kg/day pravastatin, (4) 4.0 mg/kg/day pravastatin, and (5) 16.0 mg/kg/day pravastatin.

Table X-2 below reports the data measured on the effect of pravastatin monotherapy at four different dosages on serum total cholesterol.

TABLE X-2

| | SERUM TOTAL CHOLESTEROL | | | |
|---|---|---|---|---|
| PRAVASTATIN | (mg/dL) | | | |
| DOSAGE | Pretreatment | Week 1 | Week 2 | Week 3 |
| Vehicle | 154 ± 11¹ | 164 ± 13² | 166 ± 13² | 158 ± 12 |
| | | (+6)³ | (+8) | (+3) |
| 0.25 mg/kg/day | 154 ± 12 | 157 ± 10 | 159 ± 12 | 154 ± 13 |
| | | (+2) | (+3) | (0) |
| 1.0 mg/kg/day | 155 ± 12 | 153 ± 11 | 149 ± 11² | 143 ± 10² |
| | | (−1) | (−4) | (−8) |
| 4.0 mg/kg/day | 157 ± 11 | 148 ± 10² | 146 ± 8 | 139 ± 9² |
| | | (−6) | (−7) | (−11) |
| 16.0 mg/kg/day | 155 ± 11 | 142 ± 8 | 137 ± 12² | 134 ± 13² |
| | | (−8) | (−12) | (−14) |

¹All values shown are mean ± SEM, n = 6.
²P < 0.05 versus the pretreatment serum total cholesterol value for each group by a paired, two-tailed t-test without equal variance assumption.
³( ) = % change in serum total cholesterol compared to the pretreatment value of each group.

Results

Treatment with pravastatin reduced serum total cholesterol in both a dose- and time-related manner. Unlike treatment with compound A-5 in which the maximal effect was observed after one week, treatment with the three highest doses of pravastatin resulted in reductions of serum total cholesterol that consistently dropped throughout the three-week period of the study. The lowest dose of pravastatin tested did not appear to have a significant effect at any time during the study. Serum total cholesterol concentration was decreased compared to vehicle by 2%, 1%, 6% and 8% following one week of treatment, and by 0%, 8%, 11% and 14% following three weeks of treatment, at 0.25, 1.0, 4.0 and 16.0 mg/kg/day doses of pravastatin, respectively. Although the observed differences in serum total cholesterol values following one and three weeks of treatment were not statistically different within each dosing group, the effect observed for these data appear to indicate that pravastatin requires a longer treatment time than does compound A-5 to achieve a maximal effect on cholesterol reduction.

Pravastatin did not appear to have a significant affect on fecal bile acid concentration (see Table X-3D).

### EXAMPLE 3

### Combination Therapy with Compund A-5 and Pravastatin

Beagle dogs were co-treated with compound A-5 and pravastatin (1) to examine the effect on serum total cholesterol when a combination of compound A-5 and pravastatin was administered, (2) to determine if a more potent cholesterol lowering effect could be achieved than would result if, assuming arguendo, the combined effects of the two drugs resulted in an additive lowering effect on serum total cholesterol, and (3) to determine if there was a statistically significant difference between a.m. and p.m. dosing of pravastatin.

Male beagle dogs (9-10 kg) obtained from Marshall farms were fed once a day for two hours and given water ad libitum. Prior to initiating treatment, blood samples were drawn from the cephalic vein of each dog to evaluate pretreatment total serum cholesterol levels at the start of the study.

During an initial four week dose ranging study with pravastatin (weeks 1 to 4), it was established that 3, 10 and 30 mg/kg/day of pravastatin were statistically indistinguishable in lowering serum total cholesterol 16%, 18%, and 20%, respectively. It was also determined that there was no statistically significant difference between a.m. and p.m. dosing of 10 mg/kg/day pravastatin. For the dose ranging study, the dogs were assigned to one of five groups based on mean body weights and serum total cholesterol levels. Each group received one of the following dosages: (1) vehicle (empty capsule, afternoon dosing), (2) 3.0 mg/kg/day pravastatin (afternoon dosing), (3) 10 mg/kg/day pravastatin (afternoon dosing), (4) 30 mg/kg/day pravastatin (afternoon dosing), and (5) 10 mg/kg/day pravastatin (morning dosing). One capsule containing pravastatin was administered per os to each dog between 9:00-9:30 a.m. prior to feeding for the 10 mg/kg/day morning dosing group and between 2:30-3:00 p.m. for the afternoon dosing groups. All animals were fed between 9:30-10:00 a.m. and were allowed two hours to eat, at which time any remaining food was removed. Typically, all dogs consumed their entire meal within this time period. Animals were dosed daily for four weeks and blood samples were taken at the end of each week after an overnight fast for comparison with pretreatment serum total cholesterol levels.

Following this initial four week dose-ranging study, dogs from the two groups receiving 10 mg/kg/day pravastatin (a.m. and p.m. dosing groups) were randomized into two new treatment groups based on serum total cholesterol levels to initiate the combination treatment study. One group received an empty capsule (a.m. dosing) and 10 mg/kg/day pravastatin (p.m. dosing) and the other received 4.0 mg/kg/day compound A-5 (a.m. dosing) and 10 mg/kg/day pravastatin (p.m. dosing). A third group that had received empty capsules (vehicle) in the dose-ranging study was used for compound A-5 monotherapy and was administered 4.0 mg/kg/day compound A-5 (a.m. dosing) and an empty capsule (p.m. dosing). Dosing continued in this manner for an additional four weeks (Weeks 5-8) with blood and 48-hour fecal samples collected at the end of each week for serum lipid measurements and fecal bile acid determinations, respectively. After four weeks of treatment, all dosing with compound A-5 was terminated and, except for the compound A-5 monotherapy group, pravastatin monotherapy was continued for an additional three weeks (weeks 9-11). The compound A-5 monotherapy group continued to receive empty capsules during this three week period, and blood and 48-hour fecal samples were also collected weekly during this timeframe. At the end of this three-week period, pravastatin dosing and all empty capsule dosing were discontinued and blood samples were collected weekly for a final three weeks (weeks 12-14) to monitor the return of serum total cholesterol levels back to baseline.

Serum Lipid Measurements

Blood was collected from the cephalic vein of each dog into serum separator tubes. The blood was centrifuged at 900×g for 20 minutes at room temperature and the serum decanted. All analyses were performed on a Cobas Mira Clinical Analyzer System (Roche Diagnostic Systems, Branchburg, N.J.) using Roche Diagnostic reagents for enzymatic determinations of serum cholesterol and triglycerides. Commercial calibrator and quality control materials were analyzed with each run to verify assay accuracy and precision. A two-tailed, paired Students t-test was used to determine the statistical significance of changes in total serum cholesterol in treated animals compared to their pretreatment values. A two-sample, two-tailed unequal variance Students t-test was used to determine serum total cholesterol and triglyceride changes in the combination therapy group compared to the monotherapy groups.

Table X-3A below reports the data measured in the initial dose ranging study on the effect of pravastatin monotherapy at four different dosages on total serum cholesterol.

TABLE X-3A

| PRAVASTAT | TOTAL SERUM CHOLESTEROL | | | | |
|---|---|---|---|---|---|
| IN | (mg/dL) | | | | |
| DOSAGE | Pretreatment | Week 1 | Week 2 | Week 3 | Week 4 |
| Vehicle | 139 ± 14¹ | 145 ± 13 | 139 ± 14 | 142 ± 11 | 142 ± 11 |
| | | (+4)² | (0) | (+2) | (+2) |
| 3.0 mg/kg/day | 158 ± 11 | 157 ± 16 | 133 ± 11³ | 140 ± 12³ | 132 ± 10³ |
| pm dosing | | (−1) | (−16) | (−11) | (−16) |
| 10.0 mg/kg/day | 159 ± 8 | 146 ± 10 | 129 ± 10³ | 128 ± 9³ | 126 ± 8³ |
| pm dosing⁴ | | (−8) | (−19) | (−19) | (−21) |
| 10.0 mg/kg/day | 155 ± 13 | 164 ± 12 | 135 ± 15³ | 131 ± 10³ | 130 ± 7³ |
| am dosing⁴ | | (+6) | (−13) | (−15) | (−16) |
| 30.0 mg/kg/day | 161 ± 8 | 164 ± 10 | 132 ± 8³ | 132 ± 11³ | 128 ± 7³ |
| pm dosing | | (+2) | (−18) | (−18) | (−20) |

¹All values shown are mean ± SEM, n = 6.
²( ) = % change in serum total cholesterol compared to the pretreatment value of each group.
³P < 0.05 versus the pretreatment serum total cholesterol value for each group by a paired, two-tailed t-test without equal variance assumption.
⁴The average percent reduction in serum total cholesterol for the combined 10 mg/kg/day groups (a.m. and p.m., which were statistically indistinguishable) was 18 ± 2%.

Table X-3B below reports the data measured on the effect of the compound A-5/pravastatin combination therapy on total serum cholesterol.

TABLE X-3B

| | TOTAL SERUM CHOLESTEROL | | |
|---|---|---|---|
| Week | (mg/dL) | | |
| | | | Combination |
| | | | Therapy: |
| | Compound A-5 | Pravastatin | Administration of |
| | Monotherapy: | Monotherapy: | 4.0 mg/kg/day of |
| | Administration of | Administration of | compound A-5 and |
| | 4.0 mg/kg/day of | 10 mg/kg/day of | 10 mg/kg/day of |
| | compound A-5 | pravastatin | pravastatin |
| Week 0 | 139 ± 14² | 161 ± 14 | 153 ± 5 |
| (pre- | | | |
| treat- | | | |
| ment)¹ | | | |
| Week 1 | 145 ± 12 | 157 ± 10 | 154 ± 14 |
| | (4)³ | (−2) | (1) |
| Week 2 | 139 ± 14 | 135 ± 17 | 129 ± 4 [a] |
| | (0) | (−16) | (−16) |
| Week 3 | 142 ± 11 | 132 ± 13 | 127 ± 4 [a] |
| | (2) | (−18) | (−17) |
| Week 4 | 142 ± 11 | 130 ± 9 [a] | 126 ± 5 [a] |
| | (2) | (−19) | (−18) |
| | Compound A-5 | No Compound A-5 | Compound A-5 |
| | Dosing Initiated | Dosing | Dosing Initiated |
| Week 5 | 123 ± 9 | 127 ± 9 [a] | 112 ± 10 [a] |
| | (−12) | (−21) | (−27) |
| Week 6 | 115 ± 8 [a] | 117 ± 10 [a] | 85 ± 5 [a, b, c] |
| | (−18) | (−27) | (−44) |
| Week 7 | 114 ± 6 [a] | 121 ± 7 [a] | 84 ± 3 [a, b, c] |
| | (−18) | (−25) | (−45) |
| Week 8 | 107 ± 7 [a] | 113 ± 9 [a] | 77 ± 3 [a, b, c] |
| | (−23) | (−30) | (−50) |
| | Compound A-5 | No Compound A-5 | Compound A-5 |
| | Dosing Terminated | Dosing | Dosing Terminated |
| Week 9 | 132 ± 7 | 119 ± 8 [a] | 85 ± 3 [a, b, c] |
| | (−5) | (−26) | (−44) |
| Week | 133 ± 10 | 118 ± 9 [a] | 97 ± 4 [a, b] |
| 10 | (−4) | (−27) | (−37) |
| Week | 132 ± 9 | 118 ± 8 [a] | 102 ± 3 [a, b] |
| 11 | (−5) | (−27) | (−33) |
| | No Pravastatin | Pravastatin Dosing | Pravastatin Dosing |
| | Dosing | Terminated | Terminated |
| Week | 136 ± 9 | 131 ± 9 [a] | 119 ± 5 [a] |
| 12 | (−2) | (−19) | (−22) |
| Week | 137 ± 9 | 147 ± 9 | 136 ± 6 [a] |
| 13 | (−14) | (−9) | (−11) |
| Week | Not Measured | 144 ± 12 | 139 ± 7 |
| 14 | | (−11) | (−9) |

¹During Weeks 1-4, dogs in the compound A-5 monotherapy group were treated with vehicle and dogs in the compound A-5/pravastatin and pravastatin monotherapy groups were treated with 10 mg/kg/day pravastatin. All dosing with compound A-5 was initiated at Week 5 and was terminated after Week 8. Pravastatin monotherapy was continued for an additional three weeks (through Week 11). All dosing with pravastatin was terminated after Week 11.
²All values shown are mean ± SEM, n = 6.
³( ) = % change from Week 0.
a = P < 0.05 versus week 0 serum total cholesterol value for each group by a paired, two-tailed t-test without equal variance assumption.
b = P < 0.05 versus compound A-5 monotherapy serum total cholesterol value at each time point by a two-sample, two-tailed t-test without equal variance assumption.
c = P < 0.05 versus pravastatin monotherapy serum total cholesterol value at each time point by a two-sample, two-tailed t-test without equal variance assumption.

Table X-3C below reports the data measured on the effect of the compound A-5/pravastatin combination therapy on serum triglyceride.

TABLE X-3C

| | TOTAL SERUM TRIGLYCERIDE | | |
|---|---|---|---|
| WEEK | (mg/dL) | | |
| | | | Combination |
| | | | Therapy: |
| | Compound A-5 | Pravastatin | Administration of |
| | Monotherapy: | Monotherapy: | of 4.0 mg/kg/day |
| | Administration of | Administration of | compound A-5 and |
| | of 4.0 mg/kg/day | of 10 mg/kg/day | 10 mg/kg/day of |
| | compound A-5 | pravastatin | pravastatin |
| Week 1 | Not Measured | Not Measured | Not Measured |
| Week 2 | Not Measured | Not Measured | Not Measured |
| Week 3 | Not Measured | Not Measured | Not Measured |
| Week 4¹ | 42 ± 2² | 30 ± 4 | 38 ± 3 |
| | Compound A-5 | No Compound A-5 | Compound A-5 |
| | Dosing Initiated | Dosing | Dosing Initiated |
| Week 5 | 37 ± 3 | 34 ± 3 | 28 ± 5 |
| Week 6 | 32 ± 3 | 35 ± 4 | 26 ± 3 |
| Week 7 | 36 ± 3 | 37 ± 3 | 30 ± 3 |
| Week 8 | 34 ± 3 | 38 ± 5 | 30 ± 3 |
| | Compound A-5 | No Compound A-5 | Compound A-5 |
| | Dosing | Dosing | Dosing Terminated |
| | Terminated | | |
| Week 9 | 39 ± 3 | 34 ± 2 | 26 ± 2 [a, b] |
| Week 10 | 42 ± 4 | 43 ± 5 | 32 ± 2 |
| Week 11 | 34 ± 3 | 33 ± 3 | 30 ± 2 |
| | No Pravastatin | Pravastatin Dosing | Pravastatin Dosing |
| | Dosing | Terminated | Terminated |
| Week 12 | 51 ± 5 | 46 ± 4 | 46 ± 1 |
| Week 13 | 46 ± 3 | 44 ± 3 | 49 ± 3 |
| Week 14 | Not Measured | 40 ± 4 | 35 ± 3 |

¹During Weeks 1-4, dogs in the compound A-5 monotherapy group were treated with vehicle and dogs in the compound A-5/pravastatin and pravastatin monotherapy groups were treated with 10 mg/kg/day pravastatin. Serum triglyceride determinations were not made during weeks 1-3. All dosing with compound A-5 was initiated at Week 5 and was terminated after Week 8. Pravastatin monotherapy continued for an additional three weeks and was terminated after Week 11).
²All values shown are mean ± SEM, n = 6.
a = P < 0.05 versus compound A-5 monotherapy triglyceride value at each time point by a paired, two-tailed t-test without equal variance assumption.
b = P < 0.05 versus pravastatin monotherapy triglyceride value at each time point by a paired, two-tailed t-test without equal variance assumption.

Fecal Bile Acid Measurement

Fecal samples were collected to determine the fecal bile acid concentration for each animal. Fecal collections were made for two consecutive 24-hour periods between 9:00 a.m. and 10:00 a.m. each day, prior to dosing and feeding, during the final 48 hours of the study. The separate daily collections from each dog were weighed, combined and homogenized with distilled water in a food processor to generate a homogeneous slurry. A 1.4 g aliquot of each homogenate was extracted in a final concentration of 50% tertiary butanol/distilled water (2:0.6 v/v) for 45 minutes in a 37° C. water bath and centrifuged for 13 minutes at 2000×g. The concentration of bile acids (μmoles/gram homogenate) was determined using a 96-well enzymatic assay system described in van der Meer et al., “t-Butanol Extraction of Feces: A Rapid Procedure For Enzymic Determination Of Fecal Bile Acids”, Cholesterol, Metabolism in Health and Disease: Studies in the Netherlands, edited by Beynen, et al., Ponsen and Looyen, Wageningen, 1985; and Turley et al., “Re-evaluation of the 3 Alpha-Hydroxysteroid Dehydrogenase Assay For Total Bile Acids In Bile”, J. Lipid Research, 19:924-928, 1978.

A 20 μL aliquot of each fecal extract was added to each of two sets of triplicate wells in a 96-well assay plate. A standardized sodium taurocholate solution and a standardized fecal extract solution (previously made from pooled samples and characterized for its bile acid concentration) were also analyzed for assay quality control. A standard curve for sodium taurocholate containing 30-540 nmoles/well was generated by serial dilutions of an initial 20 μL aliquot of 90 mM sodium taurocholate. A 230 μL aliquot of a reaction mixture containing 1M hydrazine hydrate, 0.1 M pyrophosphate and 0.46 mg/mL NAD was added to each well. Subsequently, a 50 μL aliquot of either 3α-hydroxysteroid dehydrogenase (“HSD”) enzyme (0.8 units/mL) or assay buffer (0.1 M sodium pyrophosphate) was then added to one each of the two sets of triplicates. Following 60 minutes of incubation at room temperature, the optical density at 340 nm was measured and the mean of each set of triplicate samples was calculated. The difference in optical density with and without HSD enzyme was used to determine the bile acid concentration (mM) of each sample based on the sodium taurocholate standard curve. The bile acid concentration of the extract (μmoles/ gram homogenate), the total weight of the fecal homogenate (grams) and the body weight of the dogs (kg) were used to calculate the corresponding fecal bile acid concentration in μmoles/kg/day for each animal.

All reagents used for the assay were obtained from Sigma Chemical Co., St. Louis, Mo. (HSD—catalog # H-1506; NAD enzyme—catalog # N1636; sodium taurocholate—catalog # T4009). A one-tailed paired Students t-Test was used to determine the statistical significance of changes in fecal bile acid concentration in treated animals compared to vehicle.

Table X-3D below reports the data measured on the effect on fecal bile acids of the compound A-5/pravastatin combination therapy.

TABLE X-3D

| | FECAL BILE ACID CONCENTRATION | | |
|---|---|---|---|
| WEEK | (μmol/kg/day) | | |
| | | | Combination Therapy: |
| | ASBT | Statin | Administration of |
| | Monotherapy: | Monotherapy: | 4.0 mg/kg/day of |
| | Administration of | Administration of | compound A-5 and |
| | 4.0 mg/kg/day of | 10 mg/kg/day of | 10 mg/kg/day of |
| | compound A-5 | pravastatin | pravastatin |
| Week 0¹ | 27 ± 6² | 37 ± 6 | 27 ± 4 |
| (Pre- | | | |
| treat- | | | |
| ment) | | | |
| Week 1 | Not Measured | Not Measured | Not Measured |
| Week 2 | Not Measured | Not Measured | Not Measured |
| Week 3 | Not Measured | Not Measured | Not Measured |
| Week 4 | Not Measured | Not Measured | Not Measured |
| | Compound A-5 | No Compound A- | Compound A-5 |
| | Dosing Initiated | 5 Dosing | Dosing Initiated |
| Week 5 | 106 ± 13³ | 17 ± 5 | 93 ± 18³ |
| | (+293)⁴ | (−54) | (+244) |
| Week 6 | 128 ± 17³ | 23 ± 7 | 107 ± 11³ |
| | (+374) | (−38) | (+296) |
| Week 7 | 183 ± 39³ | 30 ± 6 | 109 ± 16³ |
| | (+578) | (−19) | (+303) |
| Week 8 | 109 ± 23³ | 26 ± 9 | 131 ± 17³ |
| | (+304) | (−30) | (+385) |
| | Compound A-5 | | |
| | Dosing | No Compound A- | Compound A-5 |
| | Terminated | 5 Dosing | Dosing Terminated |
| Week 9 | 40 ± 6 | 28 ± 5 | 24 ± 2 |
| | (+48) | (−24) | (−11) |
| Week 10 | 43 ± 4 | 28 ± 6 | 28 ± 4 |
| | (+59) | (−24) | (+4) |

¹During Weeks 1-4, dogs in the compound A-5 monotherapy group were treated with vehicle and dogs in the compound A-5/pravastatin and pravastatin monotherapy groups were treated with 10 mg/kg/day pravastatin. All dosing with compound A-5 was initiated at Week 5 and ended after Week 8.
²All values shown are mean ± SEM, n = 6.
³P < 0.01 versus pretreatment fecal bile acid concentration for each group by a paired, one-tailed t-test without equal variance assumption.
⁴( ) = % change in fecal bile acid concentration from Week 0.

Results

There were no significant changes in body or fecal weights, stool consistency or general animal health for any of the groups throughout this study. During the initial fourweek dose-ranging period, monotherapeutic treatment with 10 mg/kg/day of pravastatin reduced (P<0.05) serum total cholesterol levels from 155±13 to 130±7 mg/dL (16% decrease) and from 159±8 to 126±8 mg/dL (21% decrease) in the a.m. and p.m. dosing groups, respectively (See Table X-3A). Following randomization of the 10 mg/kg/day a.m. and p.m. groups, continued monotherapeutic treatment with pravastatin for an additional four weeks (beginning after Week 4) reduced serum total cholesterol to 113±9 mg/dL (a 30% total decrease) compared to the pretreatment value of 161±14 mg/dL for this group (see Table X-3B).

Monotherapeutic treatment with 4 mg/kg/day of compound A-5 for four weeks (beginning after Week 4) resulted in a final 23% decrease in serum total cholesterol compared to the initial value at Week 0 (139±14 to 107±7 mg/dL).

Combination treatment with 10 mg/kg/day pravastatin and 4 mg/kg/day compound A-5 for four weeks (beginning after Week 4), however, lowered serum total cholesterol from 153±5 to 77±3 mg/dL (50% decrease) by the end of that four week period (Table X-3B). The additional reduction in serum total cholesterol observed with the combination treatment was statistically significant when compared to the reduction in serum total cholesterol observed in either the compound A-5 (P<0.05) or the pravastatin (P<0.05) monotherapy groups. Following termination of all drug therapy (Week 12), the two pravastatin groups recovered in parallel during the final three weeks of the study, indicating there was no lasting effect of compound A-5 treatment up to four weeks after its withdrawal (Table X-3B).

Accordingly, compound A-5, when administered at a dose of 4 mg/kg/day in combination with a 10 mg/kg/day dose of pravastatin, achieved a statistically significant 50% reduction in serum total cholesterol. This reduction in serum total cholesterol was markedly better than the reduction achieved by either compound A-5 monotherapy or pravastatin monotherapy. Following termination of compound A-5 administration, bile acid excretion and serum total cholesterol recovered toward the vehicle values although this recovery occurred more quickly in the monotherapy group than in the co-therapy group. Following termination of pravastatin administration, the two pravastatin-treated groups recovered in parallel toward pretreatment values for serum total cholesterol. The data from this study indicates that combination treatment with compound A-5 and pravastatin is an effective approach to significantly lower serum total cholesterol values.

For the four week period during which compound A-5 was administered, there were statistically significant (P<0.01 vs. pretreatment) increases in fecal bile acid concentration for the compound A-5 monotherapy group and the compound A-5/pravastatin combination therapy group (304% and 385% increase in fecal bile acids, respectively; see Table X-3D), indicating that administration of pravastatin in combination with compound A-5 did not impair the inhibition of apical sodium co-dependent bile acid transport by compound A-5. Moreover, fecal bile acid levels returned to their pretreatment levels within one week of the last day of dosing in both groups that had received compound A-5.

In addition, during the four-week combination dosing period, serum triglyceride (mg/dL) values were reduced in the combination treatment group when compared to either compound A-5 monotherapy or pravastatin monotherapy (see Table X-3C).

Finally, morning and afternoon dosing of pravastatin caused changes in serum total cholesterol that were statistically indistinguishable. The monotherapy testing indicates that there was not a statistical difference in the reduction of serum total cholesterol after four weeks of treatment with pravastatin whether the animals were dosed at 3 p.m. (159±8 to 126±8 mg/dL; 21% reduction) or at 8:30 a.m. (155±13 to 130±7 mg/dL; 16% reduction. According to the product label and/or the Physician's Desk Reference, most statins should be administered in the evening. For example, the conventional recommended dosing of pravastatin (for example, PRAVACHOL®), simvastatin (for example, ZOCOR®), and lovastatin (for example, MEVACOR®) is one tablet per day taken in the evening or prior to bedtime. As higher daily doses are needed, the recommendation is for two to three doses per day, usually with a higher multiple taken in the evening (e.g., 20 mg in the morning and afternoon and 40 mg in the evening for Zocor). In the subsequent combination testing (see Example 4), the administration of each drug at 8:30 a.m., even at very low doses, was effective in lowering serum cholesterol.

### EXAMPLE 4

### Combination Therapy Dosing

Beagle dogs were co-treated with compound A-5 and pravastatin to determine the effect on serum total cholesterol reduction in dogs of co-administration of different dosages of compound A-5 and pravastatin. Male beagle dogs (9-10 kg) obtained from Marshall farms were fed once a day for two hours and given water ad libitum. Prior to the initiation of treatment, the dogs were weighed and overnight fasted blood samples were drawn from the cephalic vein of each dog. Dogs were assigned to five groups (n=12/group) having similar (within 5%) cholesterol values and body weights. Each group was treated with one of the following combinations: (1) vehicle (containing no compound A-5 or pravastation), (2) 0.375 mg/kg/day compound A-5 and 0.45 mg/kg/day pravastatin, (3) 0.75 mg/kg/day compound A-5 and 0.90 mg/kg/day pravastatin, (4) 1.5 mg/kg/day compound A-5 and 1.8 mg/kg/day pravastatin, and (5) 3.0 mg/kg/day compound A-5 and 3.6 mg/kg/day pravastatin. All doses were administered per os in gelatin capsules to each dog between 8:00-8:30 a.m. prior to feeding. Animals were fed between 8:30-9:00 a.m. and were allowed two hours to eat before any remaining food was removed. Typically, most dogs had consumed their entire meal within this time period. Animals were dosed dailv for three weeks and overnight fasted blood samples were taken at the end of each week for comparison with pre-treatment serum total cholesterol levels. Three consecutive 24 hour fecal samples were collected during the last 72 hour period of the last week of treatment and used to determine the concentration of fecal bile acids in treated dogs compared to controls.

Serum Lipid Measurements

Serum lipid measurements were obtained as described in Example 3 above except that blood was collected from either the cephalic or jugular vein of each dog into serum separator tubes and the blood was centrifuged at 2000 rpm for 20 minutes. Table X-4A below reports the data measured on the effect of the compound A-5/pravastatin combination therapy on serum total cholesterol.

TABLE X-4A

| | TOTAL SERUM CHOLESTEROL | | | |
|---|---|---|---|---|
| | (mg/dL) | | | |
| DOSAGE | Pretreatment | Week 1 | Week 2 | Week 3 |
| Vehicle | 162 ± 8¹ | 153 ± 7² | 156 ± 7 | 161 ± 7 |
| | | (−6)³ | (−4) | (−1) |
| 0.375 mg/kg/day | 160 ± 8 | 132 ± 6² | 130 ± 7² | 133 ± 7² |
| compound A-5; | | (−18) | (−19) | (−17) |
| 0.45 mg/kg/day | | | | |
| pravastatin | | | | |
| 0.75 mg/kg/day | 159 ± 7 | 121 ± 5² | 121 ± 6² | 123 ± 5² |
| compound A-5; | | (−24) | (−24) | (−23) |
| 0.90 mg/kg/day | | | | |
| pravastatin | | | | |
| 1.5 mg/kg/day | 160 ± 7 | 117 ± 9² | 114 ± 6² | 116 ± 6² |
| compound A-5; | | (−27) | (−29) | (−28) |
| 1.8 mg/kg/day | | | | |
| pravastatin | | | | |
| 3.0 mg/kg/day | 161 ± 10 | 106 ± 11² | 95 ± 9² | 104 ± 10² |
| compound A-5; | | (−34) | (−41) | (−35) |
| 3.6 mg/kg/day | | | | |
| pravastatin | | | | |

¹All values shown are mean ± SEM, n = 12.
²P < 0.01 versus pretreatment serum total cholesterol value for each group by a paired, two-tailed t-test without equal variance assumption.
³“( )” indicates the % change in serum total cholesterol compared to the pretreatment value of each group.

Fecal Bile Acid Measurement

Fecal bile acid measurements were obtained as described in Example 3 above except that fecal collections were made during the final 72 hours of the study, for three consecutive 24-hour periods between 9:00 am and 10:00 am each day, prior to dosing and feeding; the mean fecal bile acid concentration of the vehicle group was subtracted from the concentration of each treatment group to determine the increase (delta value) in fecal bile acid concentration as a result of the drug treatment; and a one-tailed paired Students t-Test was used to determine the statistical significance of changes in fecal bile acid concentration in treated animals compared to vehicle. Table X-4B below reports the data measured for the effect on fecal bile acids of the compound A-5/pravastatin combination therapy.

TABLE X-4B

| | |
|---|---|
| | FECAL BILE ACID |
| | CONCENTRATION |
| DOSAGE | (μmol/kg/day) |

| Vehicle | | 22 ± 3¹ |
|---|---|---|
| 0.375 | mg/kg/day compound A-5; | 71 ± 6² |
| 0.45 | mg/kg/day pravastatin | (+222)³ |
| 0.75 | mg/kg/day compound A-5; | 94 ± 9² |
| 0.90 | mg/kg/day pravastatin | (327) |
| 1.5 | mg/kg/day compound A-5; | 105 ± 6² |
| 1.8 | mg/kg/day pravastatin | (377) |
| 3.0 | mg/kg/day compound A-5; | 104 ± 20² |
| 3.6 | mg/kg/day pravastatin | (372) |

¹All values shown are mean ± SEM, n = 12.
²P < 0.01 versus fecal bile acid concentration of the vehicle group by a two-sample, one-tailed t-test without equal variance assumption.
³“( )” = % Increase in fecal bile acid concentration compared to the vehicle group.

Results

There were no significant changes in body or fecal weights, stool consistency or general animal health for any of the groups throughout this study. Three weeks of treatment with the fixed dose combinations of 0.375 mg/kg/day compound A-5 and 0.45 mg/kg/day pravastatin; 0.75 mg/kg/day compound A-5 and 0.90 mg/kg/day pravastatin; 1.5 mg/kg/day compound A-5 and 1.8 mg/kg/day pravastatin; and 3.0 mg/kg/day compound A-5 and 3.6 mg/kg/day pravastatin, resulted in statistically significant (p<0.01) increases in fecal bile acid concentration of 222%, 327%, 377% and 372%, respectively, compared to the vehicle group. Statistically significant (p<0.01) reductions in serum total cholesterol concentration compared to pretreatment values were also observed for all doses tested. Final reductions in serum total cholesterol concentration of 17%, 23%, 28% and 35% were measured for 0.375 mg/kg/day compound A-5 and 0.45 mg/kg/day pravastatin; 0.75 mg/kg/day compound A-5 and 0.90 mg/kg/day pravastatin; 1.5 mg/kg/day compound A-5 and 1.8 mg/kg/day pravastatin; and 3.0 mg/kg/day compound A-5 and 3.6 mg/kg/day pravastatin, respectively.

### EXAMPLE 5

### Lovastatin Study

The protocol described in Example 4 for evaluating the effect of combination therapy using different dosages of compound A-5 and pravastatin was carried out using lovastatin instead of pravastatin for the following daily dose-ratios of compound A-5 to lovastatin: (1) 0.375 mg/kg/day compound A-5 and 0.45 mg/kg/day lovastatin, and (2) 1.5 mg/kg/day compound A-5 and 1.8 mg/kg/day lovastatin.

Table X-5A below reports the data measured for the effect of compound A-5/lovastatin combination therapy at these two dosages on serum total cholesterol.

TABLE X-5A

| | TOTAL SERUM CHOLESTEROL | | | | |
|---|---|---|---|---|---|
| | (mg/dL) | | | | |
| | Pretreat- | | | | |
| DOSAGE | ment | Week 1 | Week 2 | Week 3 | Week 4 |
| 0.375 mg/kg/day | 168.8 | 134.8 | 127.5 | 121.7 | 121.3 |
| compound A-5; | | (−18) | (−22) | (−25) | (−26) |
| 0.45 mg/kg/day | | | | | |
| lovastatin | | | | | |
| 1.5 mg/kg/day | 162.4 | 119.4 | 114.0 | 108.2 | 103.9 |
| compound A-5; | | (−26) | (−30) | (−33) | (−36) |
| 1.8 mg/kg/day | | | | | |
| lovastatin | | | | | |

¹All values shown are mean ± SEM, n = 12.
²P < 0.01 versus pretreatment serum total cholesterol value for each group by a paired, two-tailed t-test without equal variance assumption.
³“( )” indicates the % change in serum total cholesterol compared to the pretreatment value of each group.

Table X-5B below reports the data measured for the effect of compound A-5/lovastatin combination therapy at these two dosages on serum triglyceride.

TABLE X-5B

| | SERUM TRIGLYCERIDE | | | | |
|---|---|---|---|---|---|
| | (mg/dL) | | | | |
| | Pretreat- | | | | |
| DOSAGE | ment | Week 1 | Week 2 | Week 3 | Week 4 |
| 0.375 mg/kg/day | 51.0 | 37.7 | 37.8 | 41.8 | 31.6 |
| compound A-5; | | (−23) | (−24) | (−14) | (−37) |
| 0.45 mg/kg/day | | | | | |
| lovastatin | | | | | |
| 1.5 mg/kg/day | 45.4 | 33.4 | 38.8 | 37.2 | 28.3 |
| compound A-5; | | (−26) | (−16) | (−19) | (−39) |
| 1.8 mg/kg/day | | | | | |
| lovastatin | | | | | |

¹All values shown are mean ± SEM, n = 12.
²P < 0.01 versus pretreatment serum total cholesterol value for each group by a paired, two-tailed t-test without equal variance assumption.
³“( )” indicates the % change in serum total cholesterol compared to the pretreatment value of each group.

### EXAMPLE 6

### Atorvastatin Study

The protocol described in Example 4 for evaluating the effect of combination therapy using different dosages of compound A-5 and pravastatin was carried out using atorvastatin instead of pravastatin for the following daily dose-ratios of compound A-5 and atorvastatin: (1) 0.375 mg/kg/day compound A-5 and 0.45 mg/kg/day atorvastatin, and (2) 1.5 mg/kg/day compound A-5 and 1.8 mg/kg/day atorvastatin.

Table X-6 below reports the data measured for the effect of compound A-5/atorvastatin combination therapy at these two dosages on serum total cholesterol.

TABLE X-6

| | TOTAL SERUM CHOLESTEROL | | | | |
|---|---|---|---|---|---|
| | (mg/dL) | | | | |
| | Pretreat- | | | | |
| TREATMENT | ment | Week 1 | Week 2 | Week 3 | Week 4 |
| 0.375 mg/kg/day | 160.6 | 138.6 | 134.2 | 133.6 | 130.0 |
| compound A-5; | | (−12) | (−16) | (−14) | (−16) |
| 0.45 mg/kg/day | | | | | |
| atorvastatin | | | | | |
| 1.5 mg/kg/day | 160.8 | 120.8 | 109.8 | 108.4 | 104.8 |
| compound A-5; | | (−23) | (−29) | (−32) | (−33) |
| 1.8 mg/kg/day | | | | | |
| atorvastatin | | | | | |

¹All values shown are mean ± SEM, n = 12.
²P < 0.01 versus pretreatment serum total cholesterol value for each group by a paired, two-tailed t-test without equal variance assumption.
³“( )” indicates the % change in serum total cholesterol compared to the pretreatment value of each group.

### EXAMPLE 7

### Pharmaceutical Compositions

100 mg tablets having the composition set forth in Table X-7 can be prepared using wet granulation techniques:

TABLE X-7

| INGREDIENT | WEIGHT (mg) |
|---|---|
| Compound A-5 | 5 |
| Pravastatin | 20 |
| Lactose | 54 |
| Microcrystalline Cellulose | 15 |
| Hydroxypropyl Methyl Cellulose | 3 |
| Croscarmellose Sodium | 2 |
| Magnesium Stearate | 1 |
| Total Tablet Weight | 100 |

### EXAMPLE 8

### Pharmaceutical Compositions

100 mg tablets having the composition set forth in Table X-8 can be prepared using direct compression techniques:

TABLE X-8

| | WEIGHT |
|---|---|
| INGREDIENT | FRACTION (mg) |
| Compound A-5 | 5 |
| Pravastatin | 5 |
| Lactose | 69.5 |
| Microcrystalline Cellulose | 15 |
| Colloidal Silicon Dioxide | 0.5 |
| Talc | 2.5 |
| Croscarmellose Sodium | 2 |
| Magnesium Stearate | 0.5 |
| Total Tablet Weight | 100 |

### EXAMPLE 9

### Pharmaceutical Compositions

100 mg tablets having the composition set forth in Table X-9 can be prepared using wet granulation techniques:

TABLE X-9

| INGREDIENT | WEIGHT (mg) |
|---|---|
| Compound A-5 | 5 |
| Simvastatin | 20 |
| Lactose | 54 |
| Microcrystalline Cellulose | 15 |
| Hydroxypropyl Methyl Cellulose | 3 |
| Croscarmellose Sodium | 2 |
| Magnesium Stearate | 1 |
| Total Tablet Weight | 100 |

### EXAMPLE 10

### Pharmaceutical Compositions

100 mg tablets having the composition set forth in Table X-10 can be prepared using direct compression techniques:

TABLE X-10

| | WEIGHT |
|---|---|
| INGREDIENT | FRACTION (mg) |
| Compound A-5 | 5 |
| Simvastatin | 5 |
| Lactose | 69.5 |
| Microcrystalline Cellulose | 15 |
| Colloidal Silicon Dioxide | 0.5 |
| Talc | 2.5 |
| Croscarmellose Sodium | 2 |
| Magnesium Stearate | 0.5 |
| Total Tablet Weight | 100 |

### EXAMPLE 11

### Pharmaceutical Compositions

100 mg tablets having the composition set forth in Table X-11 can be prepared using wet granulation techniques:

TABLE X-11

| INGREDIENT | WEIGHT (mg) |
|---|---|
| Compound A-5 | 5 |
| Atorvastatin | 10 |
| Lactose | 64 |
| Microcrystalline Cellulose | 15 |
| Hydroxypropyl Methyl Cellulose | 3 |
| Croscarmellose Sodium | 2 |
| Magnesium Stearate | 1 |
| Total Tablet Weight | 100 |

### EXAMPLE 12

### Pharmaceutical Compositions

100 mg tablets having the composition set forth in Table X-12 can be prepared using direct compression techniques:

TABLE X-12

| | WEIGHT |
|---|---|
| INGREDIENT | FRACTION (mg) |
| Compound A-5 | 5 |
| Atorvastatin | 2.5 |
| Lactose | 72 |
| Microcrystalline Cellulose | 15 |
| Colloidal Silicon Dioxide | 0.5 |
| Talc | 2.5 |
| Croscarmellose Sodium | 2 |
| Magnesium Stearate | 0.5 |
| Total Tablet Weight | 100 |

Examples 13-133 relate to methods for the preparation of Compounds A-1 and A-9 and intermediates useful in the preparation of Compounds A-1 and A-9.

### EXAMPLE 13

Preparation of Phenolic Intermediate

Step 1

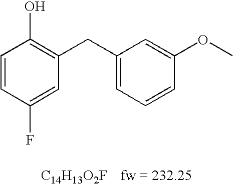

A 12-liter, 4-neck round-bottom flask was equipped with reflux condenser, N₂ gas adaptor, mechanical stirrer, and an addition funnel. The system was purged with N₂. A slurry of sodium hydride (126.0g/4.988mol) in toluene (2.5 L) was added, and the mixture was cooled to 6° C. A solution of 4-fluorophenol (560.5 g/5.000 mol) in toluene (2.5 L) was added via addition funnel over a period of 2.5 hours. The reaction mixture was heated to reflux (100 C) for Ihour. A solution of 3-methoxybenzyl chloride (783.0 g/5.000 mol) in toluene (750 mL) was added via addition funnel while maintaining reflux. After 15 hours refluxing, the mixture was cooled to room temperature and poured into H₂O (2.5 L). After 20 min. stirring, the layers were separated, and the organic layer was extracted with a solution of potassium hydroxide (720 g) in methanol (2.5 L). The methanol layer was added to 20% aqueous potassium hydroxide, and the mixture was stirred for 30 minute. The mixture was then washed 5 times with toluene. The toluene washes were extracted with 20% aqueous potassium hydroxide. All 20% aqueous potassium hydroxide solutions were combined and acidified with concentrated HCl. The acidic solution was extracted three times with ethyl ether, dried over MgSO₄, filtered and concentrated in vacuo. The crude product was purified by Kugelrohr distillation to give a clear, colorless oil (449.0 g/39% yield). b.p.: 120-130 C/50 mtorrHg. ¹H NMR and MS [(M+H)⁺=233]confirmed desired structure.

Step 2

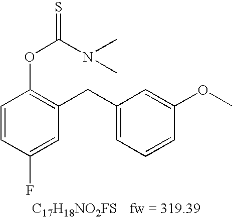

A 12-liter, 3-neck round-bottom flask was fitted with mechanical stirrer and N₂ gas adaptor. The system was purged with N₂. 4-Fluoro-2-(3-methoxybenzyl)-phenol (455.5 g/1.961 mol) and dimethylformamide were added. The solution was cooled to 6° C., and sodium hydride (55.5 g/2.197 mol) was added slowly. After warming to room temperature, dimethylthiocarbamoyl chloride (242.4 g/1.961 mol) was added. After 15 hours, the reaction mixture was poured into H₂O (4.0 L), and extracted two times with ethyl ether. The combined organic layers were washed with H₂O and saturated aqueous sodium chloride, dried over MgSO₄, filtered, and concentrated in vacuo to give the product (605.3 g, 97% yield). ¹H NMR and MS [(M+H)⁺=320] confirm desired structure.

Step 3

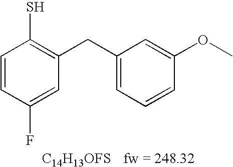

A 12-liter, round-bottom flask was equipped with N₂ gas adaptor, mechanical stirrer, and reflux condenser. The system was purged with N₂. 4-Fluoro-2-(3methoxybenzyl)-phenyldimethylthiocarbamate (605.3 g/1.895 mol) and phenyl ether (2.0kg) were added, and the solution was heated to reflux for 2 hours. The mixture was stirred for 64 hours at room temperature and then heated to reflux for 2 hours. After cooling to room temperature, methanol (2.0 L) and tetrahydrofuran (2.0 L) were added, and the solution was stirred for 15 hours. Potassium hydroxide (425.9 g/7.590 mol) was added, and the mixture was heated to reflux for 4 hours. After cooling to room temperature, the mixture was concentrated by rotavap, dissolved in ethyl ether (1.0 L), and extracted with H₂O. The aqueous extracts were combined, acidified with concentrated HCl, and extracted with ethyl ether. The ether extracts were dried (MgSO₄), filtered, and concentrated in vacuo to give an amber oil (463.0 g, 98% yield). ¹H NMR confirmed desired structure.

Step 4

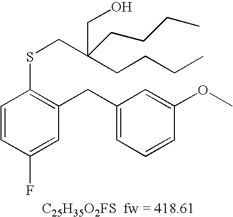

A 5-liter, 3-neck, round-bottom flask was equipped with N₂ gas adaptor and mechanical stirrer. The system was purged with N₂. 4-Fluoro-2-(3-methoxybenzyl)thiophenol (100.0 g/403.2mmol) and 2-methoxyethyl ether (1.0 L) were added and the solution was cooled to 0C. Sodium hydride (9.68 g/383.2mmol) was added slowly, and the mixture was allowed to warm to room temperature 2,2-Dibutylpropylene sulfate (110.89 g/443.6mmol) was added, and the mixture was stirred for 64 hours. The reaction mixture was concentrated by rotavap and dissolved in H₂O. The aqueous solution was washed with ethyl ether, and concentrated H₂SO₄ was added. The aqueous solution was heated to reflux for 30 minutes, cooled to room temperature, and extracted with ethyl ether. The ether solution was dried (MgSO₄), filtered, and concentrated in vacuo to give an amber oil (143.94 g/85% yield). ¹H NMR and MS [(M+H)⁺=419] confirm the desired structure.

Step 5

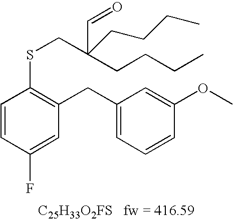

A 2-liter, 4-neck, round-bottom flask was equipped with N₂ gas adaptor, and mechanical stirrer. The system was purged with N₂. The corresponding alcohol (143.94 g/343.8 mmol) and methylene chloride (1.0 L) were added and cooled to 0° C. Pyridinium chlorochromate (140.53 g/651.6mmol) was added. After 6 hours, methylene chloride was added. After 20 minutes, the mixture was filtered through silica gel, washing with methylene chloride. The filtrate was concentrated in vacuo to give a dark yellow-red oil (1 10.6 g, 77% yield). ¹H NMR and MS [(M+H)⁺=417] confirm the desired structure.

Step 6

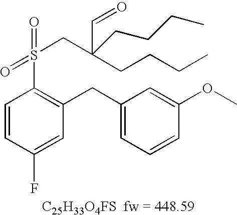

A 2-liter, 4-neck, round-bottom flask was equipped with N₂ gas adaptor and mechanical stirrer. The system was purged with N₂. The corresponding sulfide (110.6 g/265.5mmol) and methylene chloride (1.0 L) were added. The solution was cooled to 0C, and 3-chloroperbenzoic acid (158.21 g/531.7mmol) was added portionwise. After 30 minutes, the reaction mixture was allowed to warm to room temperature After 3.5 hours, the reaction mixture was cooled to 0° C. and filtered through a fine fritted funnel. The filtrate was washed with 10% aqueous K₂CO₃. An emulsion formed which was extracted with ethyl ether. The organic layers were combined, dried (MgSO₄), filtered, and concentrated in vacuo to give the product (93.2 g, 78% yield). ¹H NMR confirmed the desired structure.

Step 7

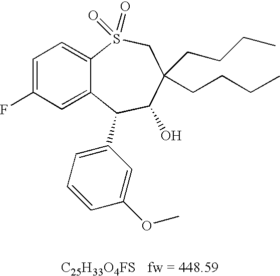

A 2-liter, 4-neck, round-bottom flask was equipped with N₂ gas adaptor, mechanical stirrer, and a powder addition funnel. The system was purged with N₂. The corresponding aldehyde (93.2 g/208mmol) and tetrahydrofuran (1.0 L) were added, and the mixture was cooled to 0° C. Potassium tert-butoxide (23.35 g/208.1 mmol) was added via addition funnel. After 1 hour, 10% aq/ HCl (1.0 L) was added. After 1 hour, the mixture was extracted three times with ethyl ether, dried (MgSO₄), filtered, and concentrated in vacuo. The crude product was purified by recrystallized from 80/20 hexane/ethyl acetate to give a white solid (32.18 g). The mother liquor was concentrated in vacuo and recrystallized from 95/5 toluene/ethyl acetate to give a white solid (33.60 g, combined yield: 71%). ¹H NMR confirmed the desired product.

Step 8

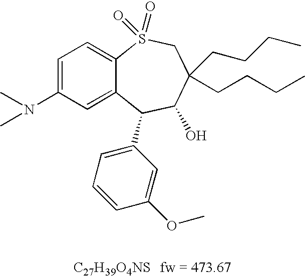

A Fisher porter bottle was fitted with N₂ line and magnetic stirrer. The system was purged with N₂. The corresponding fluoro-compound (28.1 g/62.6mmol) was added, and the vessel was sealed and cooled to −78° C. Dimethylamine (17.1 g/379mmol) was condensed via a CO₂/acetone bath and added to the reaction vessel. The mixture was allowed to warm to room temperature and was heated to 60° C. After 20 hours, the reaction mixture was allowed to cool and was dissolved in ethyl ether. The ether solution was washed with H₂O, saturated aqueous sodium chloride, dried over MgSO₄, filtered, and concentrated in vacuo to give a white solid (28.5 g/96% yield). ¹H NMR confirmed the desired structure.

Step 9

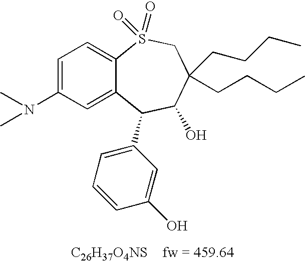

A 250-mL, 3-neck, round-bottom flask was equipped with N₂ gas adaptor and magnetic stirrer. The system was purged with N₂. The corresponding methoxy-compound (6.62 g/14.Ommol) and CHCl₃ (150 mL) were added. The reaction mixture was cooled to −78° C., and boron tribromide (10.50 g/41.9 mmol) was added. The mixture was allowed to warm to room temperature After 4 hours, the reaction mixture was cooled to 0° C. and was quenched with 10% K₂CO₃ (100 mL). After 10 minutes, the layers were separated, and the aqueous layer was extracted two times with ethyl ether. The CHCl₃ and ether extracts were combined, washed with saturated aqueous sodium chloride, dried over MgSO₄, filtered, and concentrated in vacuo to give the product (6.27 g/98% yield). ¹H NMR confirmed the desired structure.

### EXAMPLE 14

Preparation of Compound A-1

Step 1

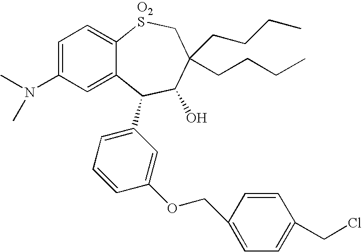

A solution of the phenol prepared in Step 9 of Example 13 (5.Og 10.89 mmol) in acetone (100 mL) at 25° C. under N₂ is treated with powdered K₂CO₃ (2.3 g, 16.3 mmoles, 1.5 equivalents) and α,α′-dichloro-p-xylene (6.7 g, 38.1 mmoles, 3.5 equivalents). The resulting solution is heated to 65° C. for 48 hours. The reaction mixture is cooled to 25° C. and concentrated. The residue is dissolved in ethyl acetate (150 mL) and washed with water (2×150 mL). The aqueous layer is extracted with ethyl acetate (2×150 mL) and the combined organic extracts are washed with saturated aqueous sodium chloride (2×150 mL). The combined extracts are dried over MgSO₄ and concentrated in vacuo. Purification by flash chromatography (SiO₂ 25%-40% ethyl acetate/hexane) affords the chlorobenzyl intermediate.

Step 2

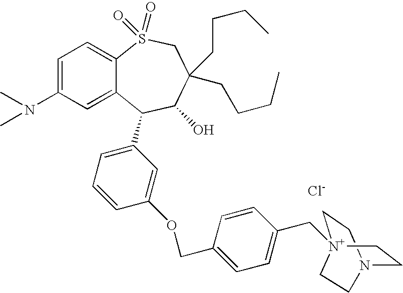

A solution of the chlorobenzyl intermediate prepared in Step 1 (4.6 g, 7.7 mmol) in acetonitrile at 25° C. under N₂ is treated with diazabicyclo[2.2.2]-octane (DABCO, 0.95 g, 8.5 mmoles, 1.1 equivalents) and stirred at 35° C. for 4 hours. After stripping off the solvent in vacuo and redissolving in minimum acetonitrile, a white solid product is obtained after precipitation.

### EXAMPLE 15

Preparation of Amine Intermediate

Step 1

Preparation of 2

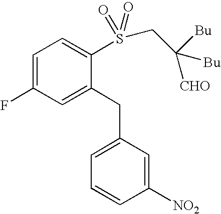

To a solution of 6.0 g of the dibutyl 4-fluorobenzene dialdehyde (14.3 mmol) prepared as described in Example 1395 of U.S. Pat. No. 5,994,391 in 72 mL of toluene and 54 mL of ethanol was added 4.7 g 3-nitrobenzeneboronic acid (28.6 mmol), 0.8 g of tetrakis (triphenylphosphine) palladium(0) (0.7 mmol) and 45 mL of a 2 M solution of sodium carbonate in water. This heterogeneous mixture was refluxed for three hours, then cooled to ambient temperature and partitioned between ethyl acetate and water. The organic layer was dried over MgSO₄ and concentrated in vacuo. Purification by silica gel chromatography (Waters Prep-2000) using ethyl acetate/hexanes (25/75) gave 4.8 g (73%) of the title compound as a yellow solid. ¹H NMR (CDCl₃) d 0.88 (t, J=7.45 Hz, 6H), 0.99-1.38 (m, 8H), 1.62-1.75 (m, 2H), 1.85-2.00 (m, 2H), 3.20 (s, 2H), 4.59 (s, 2H), 6.93 (dd, J=10.5 and 2.4 Hz, 1H), 7.15 (dt, J=8.4 and 2.85 Hz, 1H), 7.46-7.59 (m, 2H), 8.05-8.16 (m, 3H), 9.40 (s, 1H).

Step 3

Preparation of 3

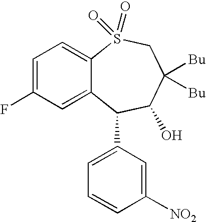

A solution of 4.8 g (10.4 mmol) of 2 in 500 mL tetrahydrofuran was cooled to 0° C. in an ice bath. A 1 M solution of potassium t-butoxide (20 mL) was added slowly, maintaining the temperature at <5° C. Stirring was continued for 30 minutes, then the reaction was quenched with 100 mL of saturated ammonium chloride. The mixture was partitioned between ethyl acetate and water; the organic layer was washed with brine, then dried (MgSO₄) and concentrated in vacuo. Purification by silica gel chromatography through a 100 ml plug using methylene chloride as eluent yielded 4.3 g (90%) of 3 as a pale yellow foam. ¹H NMR (CDCl₃) d 0.93 (t, J=7.25 Hz, 6H), 1.00-1.55 (m, 8H), 1.59-1.74 (m, 3H), 2.15-2.95 (m, 1H), 3.16 (q_{AB}, J_{AB}=15.0 Hz, ΔV=33.2 Hz, 2H), 4.17 (d, J=6.0 Hz, 1H), 5.67 (s, 1H), 6.34 (dd, J=9.6 and 3.0 Hz, 1H), 7.08 (dt, J=8.5 and 2.9 Hz, 1H), 7.64 (t, J=8.1 Hz, 1H), 7.81 (d, J=8.7 Hz, 1H), 8.13 (dd, J=9.9 and 3.6 Hz, 1H), 8.23-8.30 (m, 1H), 8.44 (s, 1H). MS(FABH⁺) m/e (relative intensity) 464.5 (100), 446.6 (65). HRMS calculated for M+H 464.1907. Found 464.1905.

Step 4

Preparation of 4

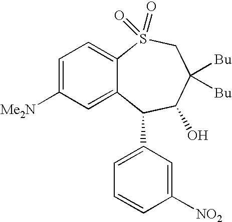

To a cooled (0° C.) solution of 4.3 g (9.3 mmol) of 3 in 30 ml tetrahydrofuran contained in a stainless steel reaction vessel was added 8.2 g dimethyl amine (182 mmol). The vessel was sealed and heated to 110 ° C. for 16 hours. The reaction vessel was cooled to ambient temperature and the contents concentrated in vacuo. Purification by silica gel chromatography (Waters Prep-2000) using an ethyl acetate/hexanes gradient (1040% ethyl acetate) gave 4.0 g (88%) of 4 as a yellow solid. ¹H NMR (CDCl₃) d 0.80-0.95 (m, 6H), 0.96-1.53 (m, 8H), 1.60-1.69 (m, 3H), 2.11-2.28 (m, 1H), 2.79 (s, 6H), 3.09 (q_{AB}, J_{AB}=15.0 Hz, DV=45.6 Hz, 2H), 4.90 (d, J=9.0 Hz, 1H), 5.65 (s, 1H), 5.75 (d, J=2.1 Hz, 1H), 6.52 (dd, J=9.6 and 2.7 Hz, 1H), 7.59 (t, J=8.4 Hz, 1H), 7.85 (d, J=7.80 Hz, 1H), 7.89 (d, J=9.0 Hz, 1H), 8.20 (dd, J=8.4 and 1.2 Hz, 1H), 8.43 (s, 1H ). MS(FABH⁺) m/e (relative intensity) 489.6 (100), 471.5 (25). HRMS calculated for M+H 489.2423. Found 489.2456.

Step 5

Preparation of 5

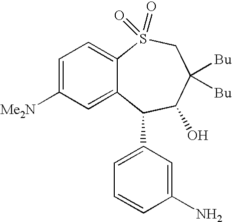

To a suspension of 1.0 g (2.1 mmol) of 4 in 100 ml ethanol in a stainless steel Parr reactor was added 1 g 10% palladium on carbon. The reaction vessel was sealed, purged twice with H₂, then charged with H₂ (100 psi) and heated to 45° C. for six hours. The reaction vessel was cooled to ambient temperature and the contents filtered to remove the catalyst. The filtrate was concentrated in vacuo to give 0.9 g (96%) of 5. ¹H NMR (CDCl₃) d 0.80-0.98 (m, 6H), 1.00-1.52 (m, 10H), 1.52-1.69 (m, 1H), 2.15-2.29 (m, 1H ), 2.83 (s, 6H), 3.07 (q_{AB}, J_{AB}=15.1 Hz, DV =44.2 Hz, 2H), 3.70 (s, 2H), 4.14 (s, 1H), 5.43 (s, 1H), 6.09 (d, J=2.4 Hz, 1H), 6.52 (dd, J=12.2 and 2.6 Hz, 1H), 6.65 (dd, J=7.8 and 1.8 Hz, 1H), 6.83 (s, 1H), 6.93 (d, J=7.50 Hz, 1H), 7.19 (t, J=7.6 Hz, 1H), 7.89 (d, J=8.9 Hz, 1H ). MS(FABH⁺) m/e (relative intensity) 459.7 (100). HRMS calculated for M+H 459.2681. Found 459.2670.

### EXAMPLE 16

Preparation of Compound A-14

Step 1

Preparation of 6

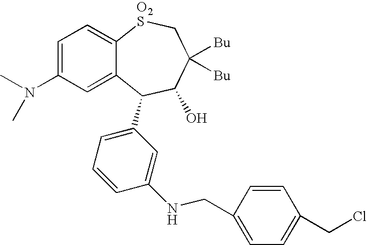

A solution of 5 prepared in Step 5 of Example 15 (5.0 g, 10.89 mmol) in acetonitrile (100 mL) at 25° C. under N₂ is treated with powdered K₂CO₃ (2.3 g, 16.3 mmoles, 1.5 equivalents) and α,α′-dichloro-p-xylene (1.9 g, 10.88 mmoles, 1.0 equivalents) and the resulting solution is heated to 65° C. for 48 hours. The reaction mixture is cooled to 25° C. and concentrated. The residue is dissolved in ethyl acetate (150 mL) and washed with water (2x150 mL). The aqueous layer is extracted with ethyl acetate (2×150 mL) and the combined organic extracts are washed with saturated aqueous sodium chloride (2x150 mL). The combined extracts are dried over MgSO₄ and concentrated in vacuo. Purification by flash chromatography (SiO₂ 25%40% ethyl acetate/hexane) affords the chlorobenzyl intermediate 6.

Step 2

Preparation of 7

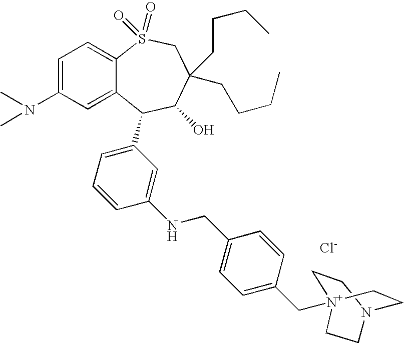

A solution of the chlorobenzyl intermediate 6 prepared in Step 1 (4.6 g, 7.7 mmol) in acetonitrile at 25° C. under N₂ is treated with diazabicyclo[2.2.2]-octane (DABCO, 0.95 g, 8.5 mmoles, 1.1 equivalents) and stirred at 35° C. for 4 hours. After stripping off the solvent in vacuo and redisolving in minimum acetonitrile, a white solid product is obtained after precipitation.

### EXAMPLE 17

### Preparation of 1-chloro-2-(4-methoxyphenyl)methyl-4-nitrobenzene, 33

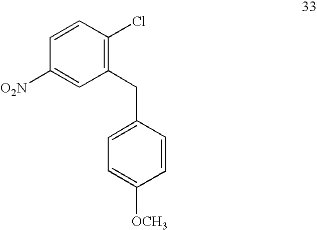

Step A. Preparation of 2-chloro-5-nitrophenyl-4′-methoxyphenyl ketone 34.

Method 1.

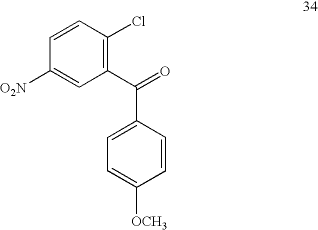

In an inert atmosphere, weigh out 68.3 g of phosphorus pentachloride (0.328 mole, Aldrich) into a 2-necked 500 mL round bottom flask. Fit the flask with a N₂ inlet adapter and suba seal. Remove from the inert atmosphere and begin N₂ purge. Add 50 mL of anhydrous chlorobenzene (Aldrich) to the PCl₅ via syringe and begin stirring with a magnetic stir bar.

Weigh out 60 g of 2-chloro-5-nitrobenzoic acid (0.298 mole, Aldrich). Slowly add the 2-chloro-5-nitrobenzoic acid to the chlorobenzene solution while under N₂ purge. Stir at room temperature overnight. After stirring at room temperature for about 20 hours, place in an oil bath and heat at 50° C. for 1 hour. Remove chlorobenzene under high vacuum. Wash the residue with anhydrous hexane. Dry the acid chloride (wt =61.95 g). Store in inert and dry atmosphere.

In an inert atmosphere, dissolve the acid chloride in 105 mL of anhydrous anisole (0.97 mole, Aldrich). Place solution in a 2-neck 500 mL round bottom flask.

Weigh out 45.1 g of aluminum trichloride (0.34 moles, Aldrich) and place in a solid addition funnel. Fit the reaction flask with an addition funnel and a N₂ inlet adapter. Remove from inert atmosphere. Chill the reaction solution with an ice bath an begin the N₂ purge. Slowly add the AlCl₃ to the chilled solution. After addition is complete, allow to warm to room temperature. Stir overnight.

Quench the reaction by pouring into a solution of 300 mL 1N HCl and ice. Stir for 15 minutes. Extract twice with ether. Combine the organic layers and extract twice with 2% NaOH, then twice with deionized H₂O. Dry over MgSO₄, filter, and rotovap to dryness. Remove the anisole under high vacuum. Crystallize the product from 90% ethanol/10% ethyl acetate. Dry on a vacuum line. Wt=35.2 g. yield 41%. Mass spec (m/z=292).

Method 2.

Change 230 kg of 2-chloro-5-nitrobenzoic acid (CNBA) to a clean dry reactor flushed with N₂. Seal the reactor and flush with N₂. To the reactor charge 460 kg of anisole. Start agitation and heat the mixture to 90° C., dissolving most of the CNBA. To the reactor charge 785 kg of polyphosphoric acid (PPA). PPA containers are warmed in a hot box (70° C.) prior to charging in order to lower viscosity. Two phases result. The upper phase contains the majority of the CNBA and anisole. The lower phase contains most of the PPA. The reaction conditions are maintained for 5 hour at which time sampling begins to determine residual CNBA. Analysis of samples is by gas chromatography. The reaction is quenched when 1.0% residual CNBA is achieved. The reaction is quenched into 796 kg H₂0. The temperature of the quenched mass is adjusted to 60° C. and maintained at this temperature until isolation. Agitation is stopped and the phases are split. The lower spent acid phase is sent to waste disposal. The upper product phase is washed with 18 kg of sodium bicarbonate in 203 kg of water, then washed with 114 kg of potable water. Agitation is stopped and the phases are split. The upper aqueous phase is sent to waste disposal. The lower product phase is cooled to about 0° C. and 312 kg of heptane is added. A mixture of ortho- and para-substituted product (total 10 kg) precipitates out of solution and is recovered by pressure filtration. To the product phase is added another 134 kg of heptane causing another 317 kg of a mixture of ortho- and para-substituted product to precipitate. The precipitate is recovered by pressure filtration. The wetcake is washed with heptane to remove residual anisole. The wetcake is dried in a rotary vacuum dryer at 60° C. Final yield of 34 is 65.1% (30.3% yield of the ortho-substituted product).

Step B. Preparation of 1-chloro-2-(4-methoxyphenyl)methyl-4-nitrobenzene, 33.

To a clean dry nitrogen purged 500 mL round bottom flask was charged 60.0 g (0.206 moles) of 34. Trifluoroacetic acid (100 grams, ca. 67 mL) was added to the reactor and the resulting suspension was heated to 30° C. to give a homogeneous wine colored solution. Next, 71.0 g (0.611 moles) of triethylsilane was placed in an addition 5 funnel and 1.7 g (0.011 moles) of trifluoromethanesulfonic acid (triflic acid) was added to reactor. The color changed from burgundy to greenish brown. Triethylsilane was added dropwise to the solution at 30° C. The batch color changed to a grass green and an exothermic reaction ensued. The exotherm was allowed to raise the batch temperature to 45° C. with minimal cooling in a water bath. The reaction temperature was controlled between 45-50° C. for the duration of addition. Addition of triethylsilane was complete in 1 hour. The batch color became greenish brown at completion. The batch was stirred for three more hours at 40° C., then allowed to cool. When the batch temperature reached ca. 30° C., product started to crystallize. The batch was further cooled to 1-2° C. in a water/ice bath, and after stirring for another half hour at 1-2° C., the slurry was filtered. The crystalline solid was washed with two 60 mL portions of hexane, the first as a displacement wash and the second as a reslurry on the filter. The solids were vacuum filtered until dry on the filter under a stream of nitrogen and the solids were then transferred to a clean container. A total of 49.9 grams of material was isolated. Mp 87.5-90.5° C. and HNMR identical with known samples of 33. GC (HP-5 25 meter column, 1 mL N₂/minute at 100° C., FID detection at 300° C., split 50:1) of the product showed homogeneous material. The isolated yield was 88% of 33.

### EXAMPLE 18

### Preparation of 2,2-dibutyl-1,3-propanediol, 54

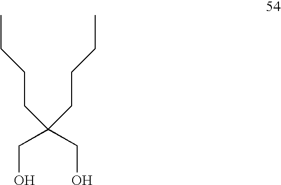

(This method is essentially the same as that described in U.S. Pat. No. 5,994,391, Example Corresponding to Scheme XI, Step 1, column 264.) Lithium aluminum hydride (662 mL, 1.2 equivalents, 0.66 mol) in 662 mL of 1M THF was added dropwise to a stirred solution of dibutyl-diethylmalonate (150 g, 0.55 mol) (Aldrich) in dry THF (700mL) while maintaining the temperature of the reaction mixture at between about −20 □C to about 0 □C using an acetone/dry ice bath. The reaction mixture was then stirred at room temperature overnight. The reaction was cooled to −20 □C and 40 mL of water, 80 mL of 10% NaOH and 80 mL of water were successively added dropwise. The resulting suspension was filtered. The filtrate was dried over sodium sulfate and concentrated under vacuum to give 98.4 g (yield 95%) of the diol as an oil. Proton NMR, carbon NMR and MS confirmed the product.

Alternate reducing agents which will be useful in this preparation of compound 54 include diisobutylaluminum hydride (DIBAL-H) or sodium bis(2-methoxyethyxy)aluminum hydride (for example, Red-Al supplied by Aldrich).

### EXAMPLE 19

### Preparation of 1 -bromo-2-butyl-2-(hydroxymethyl)hexane, 52

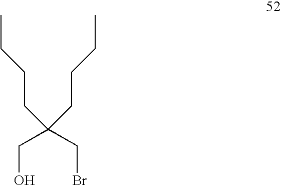

A 250 mL 3-necked round-bottomed flask was fitted with a mechanical stirrer, a nitrogen inlet, an addition funnel or condenser or distilling head with receiver, a thermocouple connected to a J-Kem temperature controller and a thermocouple connected to analog data acquisition software, and a heating mantle. The flask was purged with nitrogen and charged with 20 grams of 54. To this was added 57 grams of a 30 wt. % solution of HBr in acetic acid. The mixture was heated to 80° C. for 4 hours. The solvents were distilled off to a pot temperature of 125° C. over 20 minutes. This removes most of the residual HBr. The mixture was cooled to 80° C. and 100 mL of Ethanol 2B (source: Aaper) was added at once. Next 1.0 mL of concentrated sulfuric acid was added. The solvent was distilled off (10 to 15 mL solvent at 79-80° C.). And the mixture was refluxed for 2 h. An additional 10 to 15 mL of solvent was distilled off and the mixture was again held at reflux temperature for 2h. Further solvent was distilled off to a pot temperature of 125° C. and then the flask contents were cooled to 25.0° C. To the flask was added 100 mL of ethyl acetate and 100 mL of 2.5N sodium hydroxide. The mixture was agitated for 15 minutes and the aqueous layer was separated. Another 100 mL of water was added to the pot and the contents were agitated 15 minutes. The aqueous layer was separated and solvent was distilled off to a pot temperature of 125° C. During this process water is removed by azeotropic distillation with ethyl acetate. The product was concentrated under reduced pressure to afford 26.8 g of a brown oil containing the product 52 (96.81% by GC: HP1 column; initial temp. 50° C., hold for 2.5 minutes, Ramp 10° C./minute to ending temp. 275° C., final time 15 minutes).

### EXAMPLE 20

### Alternate Preparation of 1-bromo-2-butyl-2-hydroxymethyl)hexane, 52

A 250 mL 3-necked round-bottomed flask is fitted with a mechanical stirrer, a nitrogen inlet, an addition funnel or condenser or distilling head with receiver, a thermocouple connected to a J-Kem temperature controller and a thermocouple connected to analog data acquisition software, and a heating mantle. The flask is purged with nitrogen and charged with 20 grams of 54. To this is added 57 grams of a 30 wt. % solution of HBr in acetic acid. The mixture is heated to 80° C. for 4 hours. The solvents are vacuum distilled off to a pot temperature of 90° C. over 20 minutes. This removes most of the residual HBr. The mixture is cooled to 80° C. and 100 mL of Ethanol 2B (source: Aaper) is added at once. Next 1.0 mL of concentrated sulfuric acid is added. The solvent is distilled off (10 to 15 mL solvent at 79-80° C.). And the mixture is refluxed for 2 h. An additional 10 to 15 mL of solvent is distilled off and the mixture is again held at reflux temperature for 2h. Further solvent is distilled off to a pot temperature of 85° C. and then the flask contents are cooled to 25.0° C. To the flask is added 100 mL of ethyl acetate and 100 mL of 2.5N sodium hydroxide. The mixture is agitated for 15 minutes and the aqueous layer is separated. Another 100 mL of water is added to the pot and the contents are agitated 15 minutes. The aqueous layer is separated and solvent is distilled off to a pot temperature of 85° C. During this process water is removed by azeotropic distillation with ethyl acetate. The material is concentrated under reduced pressure to afford the product 52.

### EXAMPLE 21

### Preparation of 2-(bromomethyl)-2-butylhexanal, 53

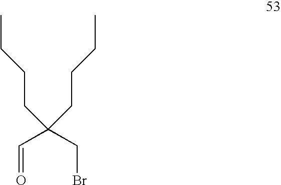

A 500 mL 3-necked round-bottom flask was fitted with a mechanical stirrer, a nitrogen inlet, an addition funnel or condenser or distilling head with receiver, a thermocouple connected to a J-Kem temperature controller and a thermocouple connected to analog data acquisition software, and a heating mantle. The flask was purged with nitrogen gas and charged with 26.0 grams of 52 and 15.6 grams of triethylamine. In a 250 mL flask was slurried 37.6 grams of sulfur trioxide-pyridine in 50 mL of DMSO. The DMSO slurry was added to the round-bottom flask by addition funnel over 15 minutes. The addition temperature started at 22° C. and reached a maximum of 41.0° C. (Addition of the slurry at temperatures below 18.0° C. will result in a very slow reaction, building up sulfur trioxide with will react rapidly when the temperature rises above 25° C.) The mixture was stirred for 15 minutes. To the mixture was added 100 mL of 2.5M HCl over 5 minutes. The temperature was maintained below 35° C. Next, 100 mL of ethyl acetate was added and the mixture was stirred 15 minutes. The mixture was then cooled to ambient and the aqueous layer was separated. To the pot was added 100 mL of water and the mixture was agitated for 15 minutes. The aqueous layer was separated. The solvent was distilled to a pot temperature of 115° C. and the remaining material was concentrated under reduce pressure to afford 21.8 g of a brown oil containing the product 53 (95.1% by GC: HP1 column; initial temp. 50° C., hold for 2.5 minutes, Ramp 10° C./minute to ending temp. 275° C., final time 15 minutes).

### EXAMPLE 22

### Alternate Preparation and Purification of 2-(Bromomethyl)-2-butylhexanal, 53

a. Preparation of Compound 52

To the reactor is charged 2,2-dibutyl-1,3-propanediol followed by 30 wt % HBr in acetic acid. The vessel is sealed and heated at an internal temperature of ca. 80° C. and held for a period of ca. 7 hours, pressure maintained below 25 psia. A GC of the reaction mixture is taken to determine reaction completion (i.e., conversion of 2,2-dibutyl-1,3-propanediol into 3-acetoxy-2,2-dibutyl-1-propanol). If the reaction is not complete at this point, the mixture may be heated for an additional period of time to complete the conversion. Acetic acid/HBr is then removed using house vacuum (ca. 25 mmHg) up to a maximum internal temperature of ca. 90° C. Ethanol is then added followed by sulfuric acid. A portion of the ethanol is removed (ca. one-quarter of the ethanol added) via atmospheric distillation. Ethanol is then added back (ca. the amount removed during the distillation) to the reactor containing the 3-acetoxy-2,2-dibutyl-1-propanol and the contents are heated to reflux (ca. 80° C. with a jacket temperature of 95° C.) and then held at reflux for ca. 8 hours. Ethanol is then removed via atmospheric distillation up to a maximum internal temperature of 85° C., using a jacket temperature of 95° C. A GC is taken to determine reaction completion (i.e., conversion of 3-acetoxy-2,2-dibutyl-1-propanol to compound 52). If the reaction is not complete, ethanol is added back to the reactor and the contents are heated to reflux and then held at reflux for an additional 4 hours (ca. 80° C., with a jacket of 95° C.). Ethanol is then removed via atmospheric distillation up to a maximum internal temperature of 85° C., using a jacket temperature of 95° C. A GC is taken to determine reaction completion (i.e., conversion of 3-acetoxy-2,2-dibutyl-1-propanol to compound 52). Once the reaction is deemed to be complete, the remaining ethanol is removed via atmospheric distillation up to a maximum internal temperature of 125° C. Methyl t-butyl ether is then added followed by a 5% sodium bicarbonate solution. The layers are separated, the aqueous layer is extracted once with MTBE, the organic extracts are combined, washed once with water, dried over MgSO₄, and concentrated under house vacuum (ca. 25 mmHg) to a maximum internal temperature of 60° C. The resultant oil is stored in the cooler until it is needed for further processing.

b. Preparation of Compound 53.

Methyl sulfoxide is charged to the reactor followed by compound 52 and triethylamine. Pyridine-sulfur trioxide complex is then added portion-wise to the reactor while maintaining an internal temperature of <35° C. Once the pyridine-sulfur trioxide complex addition is complete, a GC of the reaction mixture is taken to determine reaction completion (i.e., conversion of 52 into 53). If the reaction is not complete at this point, the mixture may be stirred for an additional period of time to complete the conversion. The reaction is quenched with an 11 wt % aqueous HCl solution. Ethyl acetate is added and the layers are separated, the aqueous layer is extracted once with ethyl acetate, the organic extracts are combined, washed once with water, dried over MgSO₄, and concentrated under house vacuum (ca. 25 mm/Hg) to a maximum internal temperature of 30° C. The resultant oil is stored in the cooler until it is needed for further processing.

c. Alternate Preparation of Compound 53.

Compound 52 and methylene chloride are charged to the reactor followed by TEMPO. The solution is cooled to ca. 0-5° C. Potassium bromide and sodium bicarbonate are dissolved in a separate reactor and added to the solution of 52 and TEMPO at 0-5° C. The biphasic mixture is cooled to 0-5° C. and sodium hypochlorite is added at such a rate to maintain an internal temperature of 0-5° C. When the add is complete a GC of the reaction mixture is performed to determine reaction completion. If the reaction is not complete (>1% 52 remaining), additional sodium hypochlorite may be added to drive the reaction to completion. Immediately after the reaction is determined to be complete, an aqueous solution of sodium sulfite is added to quench the remaining sodium hypochlorite. The layers are separated, the aqueous layer is back-extracted with methylene chloride, the combined organic fractions are washed and dried over sodium sulfate. Compound 53 is then concentrated via a vacuum distillation, up to a maximum internal temperature of ca. 30° C. The crude aldehyde is stored in the cooler until it is required for further processing.

d. Purification of Compound 53.

A Wiped Film Evaporated (WFE) apparatus is set up with the following conditions: evaporator temperature of 90° C., vacuum of ca. 0.2 mmHg and a wiper speed of 800 rpm's. The crude compound 53 is fed at a rate of 1.0-1.5 kilograms of crude per hour. The approximate ratio of product to residue during distillation is 90:10.

### EXAMPLE 23

### Preparation of 1-(2,2-dibutyl-S,S-dioxido-3-oxopropylthio)-2-((4-Methoxyphenyl)methyl)4-nitrobenzene, 30

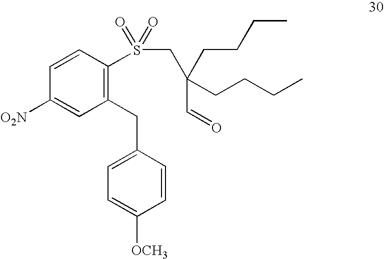

A 1000 mL 4 neck jacketed Ace flask was fitted with a mechanical stirrer, a nitrogen inlet, an addition funnel or condenser or distilling head with receiver, a thermocouple, four internal baffles and a 28 mm Teflon turbine agitator. The flask was purged with nitrogen and charged with 75.0 grams of 33. Next, the flask was charged with 315.0 grams of dimethylacetamide (DMAC), agitation was started and the mixture was heated to 30° C. Sodium sulfide (39.2 grams) was dissolved in 90 mL water in a separate flask. The aqueous sodium sulfide solution was charged into the flask over a 25 minute period. Temperature reached 37° C. at completion of addition. The solution turned dark red immediately and appeared to form a small amount of foam-like globules that adhered to the wall of the reactor. The temperature was held for two hours at 40° C. To the flask was charged 77.9 grams of 53 all at once. The reaction mixture was heated to 65° C. and held for 2 hours. Next 270 mL water was added at 65° C. The mixture was agitated 15 minutes. To the flask was then charge 315 mL of benzotrifluoride and the mixture was agitated 15 minutes. The aqueous layer was separated at 50° C. The organic layer was washed with 315 mL of 3% sodium chloride solution. The aqueous layer was separated at 50° C. The solvent was distilled to a pot temperature of 63° C. at 195 to 200 mmHg. The flask contents were cooled to 60° C. and to it was charged 87.7 grams of trimethyl orthoformate, and 5.2 grams of p-toluenesulfonic acid dissolved in 164.1 mL of methanol. The mixture was heated to reflux, 60 to 65° C. for 2 hours. The solvent was distilled to a pot temperature of 63° C. at 195 to 200 mmHg to remove methanol and methylformate. The flask was then charged with 252 mL benzotrifluoride and then cooled to 15° C. Next 22.2 grams sodium acetate as a slurry in 30 mL water was added to the flask. The flask was then charged with 256.7 grams of commercial peracetic acid (nominally 30-35% assay) over 20 minutes, starting at 15° C. and allowing the exotherm to reach 30 to 35° C. The addition was slow at first to control initial exotherm. After the first equivalent was charged the exotherm subsided. The mixture was heated to 30° C. and held for 3 hours. The aqueous layer was separated at 30° C. The organic layer was washed with 315 mL 6% sodium sulfite. The aqueous layer was separated. The flask was then charged with 40% by wt. sulfuric acid and heated to 75° C. for 2 hours. The aqueous layer was separated from the bottom at 40 to 50° C. To the flask was added 315 mL saturated sodium bicarbonate and the contents were stirred for 15 minutes. The aqueous layer was separated. The solvent was distilled to a reactor temperature of 63° C. at 195 to 200 mmHg. Next, 600 mL isopropyl alcohol was charged over 10 minutes and the temperature was maintained at 50° C. The reactor was cooled to 38° C. and held for I hour. (The product may oil slightly at first then crystallize during the hold period. If product oils out at 38° C. or does not crystallize it should be seeded to promote crystallization before cooling.) The reactor was cooled to 15° C. over 30 minutes then held for 60 minutes. The solids were filtered and dried to yield 102.1 grams of a crystalline yellow solid. Wash with 150 mL 10° C. IPA. Analysis by HPLC (Zorbax RX-C8 column, 0.1% aqueous TFA/acetonitrile gradient mobile phase, UV detection at 225 nm) showed 97.7% by weight of 30, 79.4% isolated molar corrected yield.

### EXAMPLE 23A

Alternate Preparation of 1-(2,2-dibutyl-S,S-dioxido-3-oxopropylthio)-2-((4-methoxyphenyl)methyl)4-nitrobenzene, 30

Step 1. Preparation of sulfide aldehyde compound 69.

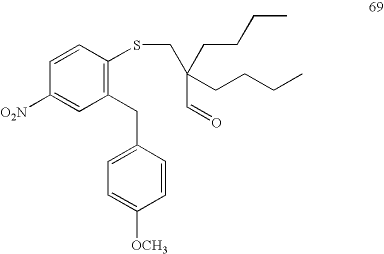

A 1000 mL 4 neck jacketed Ace reator is fitted with a mechanical stirrer, nitrogen inlet, additional funnel, a thermocouple, four internal baffles, and a 28 mm Teflon turbine agitator. The flask is purged with nitrogen gas and charged with 145 g of compound 33 and 609 mL of N,N-dimethylacetamide (DMAC). Agitation is started and the mixture is heated to 30° C. In a separate flask 72.3 g of Na₂S (Spectrum) is dissolved in 166.3 mL of water. The aqueous Na₂S is charged to the flask over a period of about 90 minutes. Addition rate should be adjusted to maintain the reaction temperature below 35° C. The mixture is stirred at 35° C. for 2 hours and then 150.7 g of compound 53 is added all at once. The mixture is heated to 70° C. and held for 2 hours. To the mixture is adjusted to 50° C., to it is added 442.7 mL water and the mixture is agitated for 15 minutes. To the reactor is then charged 609 mL of benzotrifluoride followed by 15 minutes of agitation. The aqueous layer is separated at 50° C. The organic layer is washed with 3% aqueous NaCl. The aqueous layer is separated at 50° C. The organic layer contains compound 69. The organic layer is stable and can be held indefinitely.

Step 2. Preparation of Compound 70.

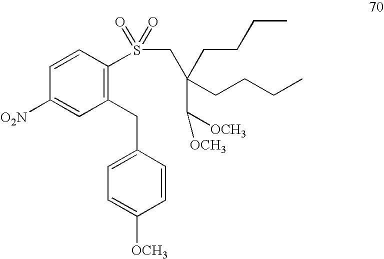

The solvent is distilled at about 63° C. to 66° C. and 195 to 200 mmHg from the organic layer resulting from Step 1 until a third to a half of the benzotrifluoride volume is distilled. The mixture is cooled to about 60° C. and charged with 169.6 g of trimethylorthoformate and about 10 g of p-toluenesulfonic acid dissolved in 317.2 mL of methanol. (Note: alternate orthoformates, for example triethylorthoformate, can be used in place of trimethylorthoformate to obtain other acetals.) The reactor is fitted with a condenser and a distillation head. The mixture is heated to boiling and from it is distilled 5 mL of methanol to remove residual water from the condenser and the mixture is held at reflux at 60° C. to 65° C. for about 2 hours. Solvent is then distilled to a pot temperature of 60° C. to 66° C. at 195 to 200 mm Hg to remove methanol and methylformate. To the mixture is added 355.4 mL benzotrifluoride and the mixture is cooled to 15° C. To the reactor is charged 32.1 g sodium acetate slurried in 77.2 mL water. The reaction is held for 72 hours. To the reactor is then charged 340.4 g of peracetic acid over a 2 hour period starting at 1 5° C. Addition was adjusted to keep the temperature at or below 20° C. The mixture was then heated to 25° C. for 4 hours. The aqueous (top) layer was separated at 25° C. and the organic layer was washed with 190 mL of 10% sodium sulfite. The organic layer contains compound 70 and can be stored indefinitely.

Step 3. Preparation of Compound 30.

To the organic layer of Step 2 is added 383.8 g of concentrated sulfuric acid. The mixture is heated at 75° C. for 2 hours and the aqueous (bottom) layer is separated at 40 to 50° C. To the reactor is charged 609 mL of 10% sodium bicarbonate and the mixture is stirred for 15 minutes. The aqueous (top) layer is separated. Solvent is distilled from the organic layer at 63 to 66° C. at 195 to 200 mm Hg. To the reactor is charged 1160 mL of isopropyl alcohol over 10 minutes at 50° C. The reactor is cooled to 38° C. and held for 1 hour. Some crystallization occurs. The reactor is cooled to 15° C. over 30 minutes and held for 120 minutes, causing further crystallization of 30. The crystals are filtered and dried to yield 200.0 g of a crystalline yellow solid. The crystals of 30 are washed with 290 mL of 10C isopropyl alcohol.

### EXAMPLE 24

### Preparation of 1-(2,2-dibutyl-S,S-dioxido-3-oxopropylthio)-2-((4-methoxyphenyl)methyl)-4-dimethylaminobenzene, 29.

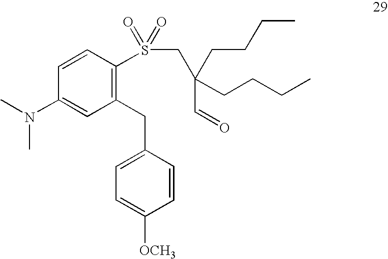

A 300 mL autoclave was fitted with a Stirmix hollow shaft gas mixing agitator, an automatic cooling and heating temperature control, and an in-reactor sampling line with sintered metal filter. At 20° C. the autoclave was charged with 15.0 grams of LO, 2.5 grams of Pd/C catalyst, 60 grams of ethanol, 10.0 grams of formaldehyde (36% aqueous solution), and 0.55 grams of concentrated sulfuric acid. The reactor was closed and pressurized the reactor to 60 psig (515 kPa) with nitrogen to check for leakage. The pressure was then reduced to 1-2 psig (108 - 115 kPa). The purge was repeated three times. The autoclave was then pressurized with H₂ to 60 psig (515 kPa) while the reactor temperature was held at 22° C. The agitator was started and set to 800-1000 rpm and the reactor temperature control is set at 30-40° C. When the cooling capacity was not enough to control the temperature, the agitator rpm or the reactor pressure was reduced to maintain the set temperature. After about 45 minutes when the heat release was slowing down (about 70% of hydrogen usage was reacted), the temperature was raised to 60° C. Hydrogen was then released and the autoclave was purged with nitrogen three times. The content of the reactor was pressure filtered through a sintered metal filter at 60° C. The filtrate was stirred to cool to the room temperature over 1-2 hours and 50 grams of water was added over 1 hour. The mixture was stirred slowly at 4° C. overnight and filtered through a Buche type filter. The cake was air dried to give 13.0 grams of 29 with 99+% assay. The isolated yield was 89%.

### EXAMPLE 25

### Preparation of syn-3,3-dibutyl-7-(dimethylamino)-1,1-dioxido4-hydroxy-5-(4-methoxyphenyl)-2,3,4,5-tetrahydrobenzothiepine, syn-24

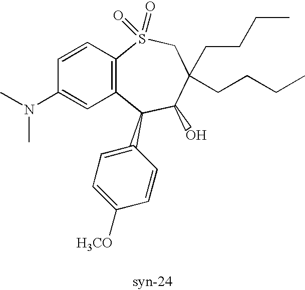

A 250 mL round bottom glass reactor fitted with mechanical agitator and a heating/cooling bath was purged with nitrogen. Forty-five grams of potassium tbutoxide/THF solution were charged to the reactor and agitation was started. In a separate container 18 grams of 29 was dissolved in 25 grams of THF. The 29/THF solution was charged into the reactor through a addition funnel over about 2.0 hours. The reactor temperature was controlled between about 16-20° C. Salt precipitated after about half of 29 was added. The slurry was stirred at 16-20° C. for an hour. The reaction was quenched with 54 grams of 7.4% ammonium chloride aqueous solution over a period of about 30 minutes while keeping the reactor temperature at 16-24° C. The mixture was gently stirred until all salt is dissolved (about 10 minutes). Agitation was stopped and the phases were allowed to separate. The aqueous layer was drained. The organic layer was charged with 50 mL water and 25 grams of isopropyl alcohol. The agitator was started and crystallization was allowed to take place. The THF was distilled under the ambient pressure, with b.p. from 60 to 65° C. and pot temperature from 70 to 77° C. The crystals dissolved as the pot gets heated and reappeared when the THF started to distill. After distillation was complete, the slurry was slowly cooled to 4° C. over 2-3 hours and stirred slowly for several hours. The slurry was filtered with a 150 mL Buche filter and the cake was washed with 10 grams of cold 2:1 water/isopropyl alcohol solution. Filtration was complete in about 5 minutes. The cake was air dried to give 16.7 grams of syn-24 with 99+% assay and a 50/50 mixture of R,R and S,S isomers.

### EXAMPLE 26A

### Conditions for Optical Resolution of Compound (4R,5R)-24

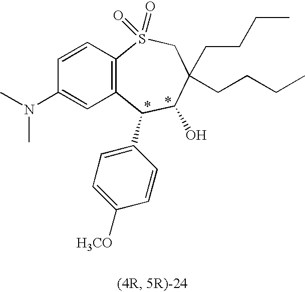

The following simulated moving bed chromatography (SMB) conditions are used to separate the (4R,5R) and (4S,5S) enantiomers of compound syn-24.

| Column (CSP): | Daicel Chiralpak AS | |
|---|---|---|
| Mobile Phase: | acetonitrile (100%) | |
| Column Length: | 11 | cm (9 cm for column 6) |
| Column I.D.: | 20.2 | cm |
| Number of Columns: | 6 | columns |
| Feed Concentration: | 39 | grams/liter |
| Eluent Flowrate: | 182 | L/hour |
| Feed Flowrate: | 55 | L/hour |
| Extract Flowrate: | 129.4 | L/hour |
| Raffinate Flowrate: | 107.8 | L/hour |
| Recycling Flowrate: | 480.3 | L/hour |
| Period: | 0.6 | minute |

| | |
|---|---|
| Temperature: | ambient |

SMB performance:
Less retained enantiomer purity (%): 92.8%
Less retained enantiomer concentration: 10 g/L
More retained enantiomer recovery yield (%): 99.3%
More retained enantiomer concentration: 7 g/L

### EXAMPLE 26B

### Alternate Conditions for Optical Resolution of Compound (4R,5R)-24

The following simulated moving bed chromatography (SMB) conditions are used to separate the (4R,5R) and (4S,5S) enantiomers of compound syn-24.

| Column (CSP): | di-methyl phenyl derivative of | |
|---|---|---|
| | tartaric acid (Kromasil DMB) | |
| Mobile Phase: | toluene/methyl tert- | |
| | butyl ether (70/30) | |
| Column Length: | 6.5 | cm |
| Column I.D.: | 2.12 | cm |
| Number of Columns: | 8 | columns |
| Zones: | 2-3-2-1 | |
| Feed Concentration: | 6.4 | weight percent |
| Eluent Flowrate: | 20.3 | g/minute |
| Feed Flowrate: | 0.7 | g/minute |
| Extract Flowrate: | 5.0 | g/minute |
| Raffinate Flowrate: | 16.0 | g/minute |
| Period: | 8 | minute |

| | |
|---|---|
| Temperature: | ambient |

SMB performance: Less retained enantiomer purity (%): >98%
Less retained enantiomer recovery yield (%): >95%

### EXAMPLE 26C

### Alternate Conditions for Optical Resolution of Compound (4R,5R)-24

The following simulated moving bed chromatography (SMB) conditions are used to separate the (4R,5R) and (4S,5S) enantiomers of compound syn-24.

| Column (CSP): | di-methyl phenyl derivative of | |
|---|---|---|
| | tartaric acid (Kromasil DMB) | |
| Mobile Phase: | toluene (100%) | |
| Column Length: | 6.5 | cm |
| Column I.D.: | 2.12 | cm |
| Number of Columns: | 8 | columns |
| Zones: | 2-3-2-1 | |
| Feed Concentration: | 64 | weight percent |
| Eluent Flowrate: | 20.3 | g/minute |
| Feed Flowrate: | 0.5 | g/minute |
| Extract Flowrate: | 4.9 | g/minute |
| Raffinate Flowrate: | 15.9 | g/minute |
| Period: | 8 | minute |

| | |
|---|---|
| Temperature: | ambient |

SMB performance: Less retained enantiomer purity (%): >98%
Less retained enantiomer recovery yield (%): >95%

### EXAMPLE 26D

### Racemization of Compound (4S,5S)-24

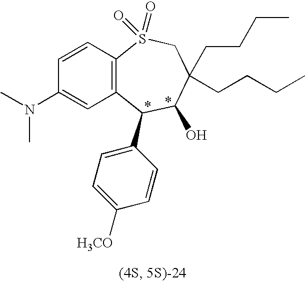

A 250 mL round bottom glass reactor with mechanical agitator and a heating/cooling bath is purged with nitrogen gas. In a flask, 18 g of (4S,5S)-24 (obtained as the more retained enantiomer in Examples 26A-26C) is dissolved in 50 g of dry THF. This solution is charged into the reactor and brought to about 23-25° C. with agitation. To the reactor is charged 45 g of potassium t-butoxide/THF solution (1 M, Aldrich) through an addition funnel over about 0.5 hour. A slurry forms. Stir the slurry at about 24-26° C. for about 1-1.5 hours. The reaction is quenched with 54 g of 7.5% aqueous ammonium chloride while keeping the reactor temperature at about 23-26° C. The first ca. 20% of the ammonium chloride solution is charged slowly until the slurry turns thin and the rest of the ammonium chloride solution is charged over about 0.5 hour. The mixture is stirred gently until all the salt is dissolved. The agitation is stopped and the phases are allowed to separate. The aqueous layer is removed. To the organic layer is charged 50 mL of water and 25 g of isopropyl alcohol. The agitator is started and crystallization is allowed to take place. THF is removed by distillation at ambient pressure. The crystals dissolve as the pot warms and then reappear when the THF starts to distill. The resulting slurry is cooled slowly to 4° C. within 2-3 hours and slowly stirred for 1-2 hours. The slurry is filtered with a 150 mL Buche filter and washed with 20 g of 0-4° C. isopropyl alcohol. The cake is air dried at about 50-60° C. under vacuum to give 16.7 g of racemic 24.

### EXAMPLE 27

### Preparation of (4R,5R)-3,3-dibutyl-7-(dimethylamino)-1,1-dioxido4-hydroxy-5-(4-hydroxyphenyl)-2,3,4,5-tetrahydrobenzothiepine, (4R,5R)-28

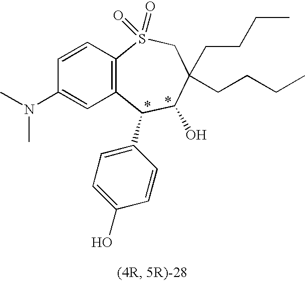

A 1000 mL 4 neck Reliance jacketed reactor flask was fitted with a mechanical stirrer, a nitrogen inlet, an addition funnel, condenser or distillation head with receiver, a thermocouple, and a Teflon paddle agitator. The flask was purged with nitrogen gas and was charged with 41.3 grams of (4R,5R)-24 and 18.7 grams of methionine followed by 240 grams of methanesulfonic acid. The mixture was heated to 75° C. and stirred for 8 hours. The mixture was then cooled to 25° C. and charged with 480 mL of 3-pentanone. The solution was homogeneous. Next, the flask was charged with 320 mL of dilution water and was stirred for 15 minutes. The aqueous layer was separated and to the organic layer was added 250 mL of saturated sodium bicarbonate. The mixture was stirred for 15 minutes and the aqueous layer was separated. Solvent was distilled to approximately one-half volume under vacuum at 50° C. The flask was charged with 480 nL of toluene, forming a clear solution. Approximately half the volume of solvent was removed at 100 mmHg. The mixture was cooled to 10° C. and stirred overnight. Crystals were filtered and washed with 150 mL cold toluene and allowed to dry under vacuum. Yielded 29.9 g with a 96.4 wt % assay. The filtrate was concentrated and toluene was added to give a second crop of 2.5 grams of crystals. A total of 32.1 g of dry off white crystalline (4R,5R)-28 was obtained.

The examples below illustrate the use of the (4R,5R)-28 product in the preparation of the (4R,5R)-configuration of Compound A-5. This (4R,5R)-28 product likewise could be used as an intermediate in the preparation of, for example, Compounds A-2, A-3, A-4, A-7, A-12 and A-13.

### EXAMPLE 27A

### Alternate Preparation of (4R,5R)-3,3-dibutyl-7-(dimethylamino)- 1,1 -dioxido4-hydroxy-5-(4-hydroxyphenyl)-2,3,4,5-tetrahydrobenzothiepine,(4R,5R)-28

A 1000 mL 4 neck Ace jacketed reactor flask is fitted with a mechanical stirrer, a nitrogen inlet, an addition funnel, condenser or distillation head with receiver, a thermocouple, and a Teflon paddle agitator. The flask is purged with nitrogen gas and is charged with 40.0 grams of (4R,5R)-24 and 17.8 grams of methionine followed by 178.6 grams of methanesulfonic acid. The mixture is heated to 80° C. and stirred for 12 hours. The mixture is then cooled to 15° C. and charged with 241.1 mL of water over 30 minutes. The reactor is then charged with 361.7 mL of 3-pentanone. Next, the flask is stirred for 15 minutes. The aqueous layer is separated and to the organic layer is added 361.7 mL of saturated sodium bicarbonate. The mixture is stirred for 15 minutes and the aqueous layer was separated. Solvent is distilled to approximately one-half volume under vacuum at 50° C. Crystals start to form at this time. The flask is charged with 361.7 nL of toluene and the mixture is cooled to 0° C. Crystals are allowed to form. Crystals are filtered and washed with 150 mL cold toluene and allowed to dry under vacuum at 50° C. Yield 34.1g of off-white crystalline (4R,5R)-28.

### EXAMPLE 27B

### Alternate preparation of (4R,5R)-3,3-dibutyl-7-(dimethylamino)- 1,1-dioxido-4-hydroxy-5-(4-hydroxyphenyl)-2,3,4,5-tetrahydrobenzothiepine, (4R,5R)-28

A first 45 L reactor is purged with nitrogen gas. To it is charged 2.5 kg of (4R,5R)-24 followed by 1.1 kg of methionine and 11.1 kg of methanesulfonic acid. The reaction mixture is heated to 85° C. with agitation for 7 hours. The reaction mixture is then cooled to 5° C. and 17.5 L of water is slowly charged to the first reactor. The reaction temperature will reach about 57° C. Next, 17.5 L of methyl isobutyl ketone (MBK) are charged to the first reactor and the reaction mixture is stirred for 30 minutes. The mixture is allowed to stand for 30 minutes and the layers are separated. The aqueous phase is transferred to a second 45 L reactor and 10 L of MIBK is charged to the second reactor. The second reactor and its contents are stirred for 30 minutes and then allowed to stand for 30 minutes while the layers separate. The organic phase is separated from the second reactor and the two organic phases are combined in the first reactor. To the first reactor is carefully charged 1.4 kg of aqueous sodium bicarbonate. The mixture is stirred for 30 minutes and then allowed to stand for 30 minutes. The phases are separated. If the pH of the aqueous phase is less than 6 then a second bicarbonate wash is performed. After the bicarbonate wash, 15 L of water is charged to the first reactor and the mixture is heated to 40° C. The mixture is stirred for 30 minutes and then allowed to stand for 30 minutes. The phases are separated. The organic phase is concentrated by vacuum distillation so that approximately 5 L of MIBK remain in the concentrate. The distillation starts when the batch temperature is at 35° C. at 1 psia. The distillation is complete when the batch temperature reaches about 47.8° C. The batch temperature is then adjusted to 45° C. and 20 L of heptane is charged to the product mixture over 20 minutes. The resulting slurry is cooled to 20° C. The product slurry is filtered (10 micron cloth filter) and washed with 8 L of 20% MIBK/heptane solution. The product is dried on the filter at 80° C. for 21 hours under vacuum. A total of 2.16 kg of white crystalline (4R,5R)-28 is isolated.

### EXAMPLE 27C

### Batch Isolation of Compound (4R,5R)-28 (or Compound (4S,5S)-28) from Acetonitrile Solution

A 1 L reactor is equipped with baffles and a 4-blade radial flow turbine. The reactor is purged with 1 L of nigrogen gas and charged with 300 mL of water. The water is stirred at a minimum rate of 300 rpm at 5° C. The reactor is charged with 125-185 mL of (4R,5R)-28 in acetonitrile solution (20% w/w) at a rate of 1.4/minute. Upon addition, crystals start to form. After addition of the acetonitrile solution, crystals are filtered through a Buchner funnel. The cake is washed with 3 volumes of water and/or followed by 1-2 volumes of ice cold isopropyl alcohol before drying. Alternatively, this procedure can be used on an acetonitrile solution of (4S,5S)-28 to isolate (4S,5S)-28.

### EXAMPLE 27D

### Continuous Isolation of Compound (4R,5R)-28 (or Compound (4S,5S)-28) from Acetonitrile Solution

A 1 L reactor is equipped with baffles and a 4-blade radial flow turbine. The reactor is purged with IL of nigrogen gas and charged with 60 grams of water and 30 grams of acetonitrile. The mixture is stirred at 300 rpm and 5° C. Into the reactor are fed 300 mL of water and 125 mL of 20% (w/w) (4R,5R)-28 in acetonitrile solution at rates of 1.7 mUminute and 1 mUminute, respectively. When the contents of the reactor reach 70-80% of the volume of the reactor, the slurry can be drained to a filter down to aminimum stirring level in the reactor and followed by more feeding. Alternatively, the reactor can be drained continuously as the feeds continue. The water/acetonitrile ratio can be in the range of about 2:1 to about 3:1. Filtered cake can be handled as described in Example 27C. Alternatively, this procedure can be used on an acetonitrile solution of (4S,5S)-28 to isolate (4S,5S)-28.

### EXAMPLE 28

Preparation of 1-(chloromethyl)-4-(hydroxymethyl)benzene, 55

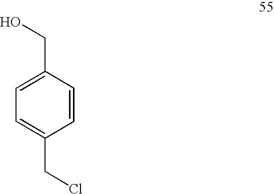

A reaction flask fitted with a nitrogen inlet and outlet, a reflux condenser, and a magnetic stirrer was purged with nitrogen. The flask was charged with 25 g of 4-(chloromethyl)benzoic acid. The flask was charged with 75 mL of THF at ambient temperature. Stirring caused a suspension to form. An endothermic reaction ensued in which the temperature of the reaction mixture dropped 22° C. to 14° C. To the reaction mixture 175 mL of borane-TBF adduct was added via a dropping funnel over about 30 minutes. During this exothemic addition, an ice-bath was used for external cooling to keep the temperature below 30° C. The reaction mixture was stirred at 20° C. for 1 hour and it was then cooled to 0° C. The reaction mixture was quenched by slow addition of 1M sulfuric acid. The resulting reaction mixture was diluted with 150 mL of t-butyl methyl ether (TBME) and stirred for at least 20 minutes to destroy boric acid esters. The layers were separated and the aqueous layer was washed with another portion of 50 mL of TBME. The combined organic layers were washed twice with 100 mL of saturated sodium bicarbonate solution. The organic layer was dried over 11 g of anhydrous sodium sulfate and filtered. The solvents were evaporated on a rotary evaporator at 45° C. (bath temperature) and <350 mbar yielding a colorless oil. The oil was seeded with crystals and the resulting solid 55 was dried under vacuum. Yield: 19.7 g/86%). Assay by GC (HP-5 25 meter column, 1 mL N₂/minute at 100° C., FID detection at 300° C., split 50:1).

### EXAMPLE 29

### Preparation of (4R,5R)-1-((4-(4-(3,3-dibutyl-7-(dimethylamino)-2,3,4,5-tetrahydro-4-hydroxy-1,1-dioxido-1-benzithiepin-5-yl)phenoxy)methyl)phenyl)methyl-4-aza-1-azoniabicyclo[2.2.2]octane chloride, 41

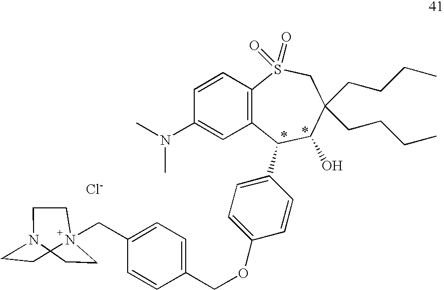

Step 1. Preparation of (4R,5R)-26.

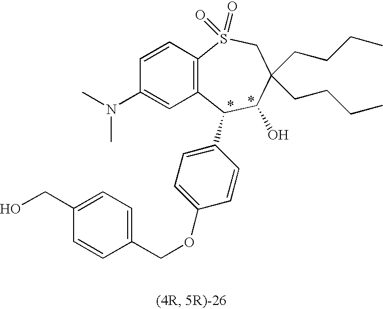

A 1000 mL 4 neck jacketed Ace reactor flask was fitted with a mechanical stirrer, a nitrogen inlet, an addition funnel or condenser or distilling head with receiver, a thermocouple, four internal baffles and a 28 mm Teflon turbine agitator. The flask was purged with nitrogen gas and charged with 25.0 grams of (4R,5R)-28 and 125 mL of N,N-dimethylacetamide (DMAC). To this was added 4.2 grams of 50% sodium hydroxide. The mixture was heated to 50° C. and stirred for 15 minutes. To the flask was added 8.3 grams of 55 dissolved in 10 mL of DMAC, all at once. The temperature was held at 50° C. for 24 hours. To the flask was added 250 mL of toluene followed by 125 mL of dilution water. The mixture was stirred for 15 minutes and the layers were then allowed to separate at 50° C. The flask was then charged with 125 mL of saturated sodium chloride solution and stirred 15 minutes. Layers separated cleanly in 30 seconds at 50° C. Approximately half of the solvent was distilled off under vacuum at 50° C. The residual reaction mixture contained (4R,5R)-26.

Step 2. Preparation of (4R,5R)-27.

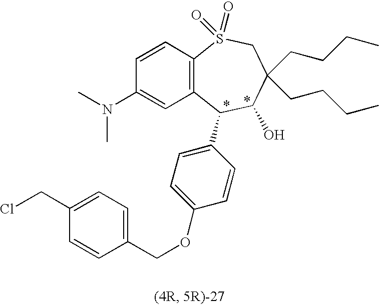

Toluene was charged back to the reaction mixture of Step 1 and the mixture was cooled to 35° C. To the mixture was then added 7.0 grams of thionyl chloride over 5 minutes. The reaction was exothermic and reached 39° C. The reaction turned cloudy on first addition of thionyl chloride, partially cleared then finally remained cloudy. The mixture was stirred for 0.5 hour and was then washed with 0.25N NaOH. The mixture appeared to form a small amount of solids which diminished on stirring, and the layers cleanly separated. The solvent was distilled to a minimum stir volume under vacuum at 50° C. The residual reaction mixture contained (4R,5R)-27.

Step 3. Preparation of 41.

To the reaction mixture of Step 2 was charged with 350 mL of methyl ethyl ketone (MEK) followed by 10.5 mL water and 6.4 grams of diazabicyclo[2.2.2]octane (DABCO) dissolved in 10 mL of MEK. The mixture was heated to reflux, and HPLC showed <0.5% of (4R,5R)-27. The reaction remained homogenous initially then crystallized at the completion of the reaction. An additional 5.3 mL of water was charged to the flask to redissolve product. Approximately 160 mL of solvent was then distilled off at atmospheric pressure. The mixture started to form crystals after 70 mL of solvent was distilled. Water separated out of distillate indicating a ternary azeotrope between toluene, water and methyl ethyl ketone (MEK). The mixture was then cooled to 25° C. The solids were filtered and washed with 150 mL MEK, and let dry under vacuum at 60° C. Isolated 29.8.0 g of off-white crystalline 41.

### EXAMPLE 29A

### Alternate Preparation of (4R,5R)-1-((4-(4-(3,3-dibutyl-7-(dimethylamino)-2,3,4,5-tetrahydro4-hydroxy-1,1-dioxido-1-benzithiepin-5-yl)phenoxy)methyl)phenyl)methyl-4aza-1-azoniabicyclo[2.2.2]octane chloride, Form II of 41

A 1000 mL 4 neck jacketed Ace reactor flask is fitted with a mechanical stirrer, a nitrogen inlet, an addition funnel or condenser or distilling head with receiver, a thermocouple, four internal baffles and a 28 mm Teflon turbine agitator. The flask is purged with nitrogen gas and charged with 25.0 grams of (4R,5R)-28 and 100 mL of N,N-dimethylacetamide (DMAC). The mixture is heated to 50° C. and to it is added 4.02 grams of 50% sodium hydroxide. The mixture is stirred for 30 minutes. To the flask is added 8.7 grams of 55 dissolved in 12.5 mL of DMAC, all at once. The charge vessel is washed with 12.5 mL DMAC and the wash is added to the reactor. The reactor is stirred for 3 hours. To the reactor is added 0.19 mL of 49.4% aqueous NaOH and the mixture is stirred for 2 hours. To the mixture is added 0.9 g DABCO dissolved in 12.5 mL DMAC. The mixture is stirred 30 to 60 minutes at 50° C. To the flask is added 225 mL of toluene followed by 125 mL of dilution water. The mixture is stirred for 15 minutes and the layers are then allowed to separate at 50° C. The bottom aqueous layer is removed but any rag layer is retained. The flask is then charged with 175 mL of 5% hydrochloric acid solution and stirred 15 minutes. Layers are separated at 50° C. to remove the bottom aqueous layer, discarding any rag layer with the aqueous layer. Approximately half of the solvent is distilled off under vacuum at a maximum pot temperature of 80° C. The residual reaction mixture contains (4R,5R)-26.

Step 2. Preparation of (4R,5R)-27.

Toluene (225 mL) is charged back to the reaction mixture of Step 1 and the mixture is cooled to 30° C. To the mixture is then added 6.7 grams of thionyl chloride over 30 to 45 minutes. The temperature is maintained below 35° C. The reaction turns cloudy on first addition of thionyl chloride, then at about 30 minutes the layers go back together and form a clear mixture. The mixture is stirred for 0.5 hour and is then charged with 156.6 mL of 4% NaOH wash over a 30 minute period. The addition of the wash is stopped when the pH of the mixture reaches 8.0 to 10.0. The bottom aqueous layer is removed at 30° C. and any rag layer is retained with the organic layer. To the mixture is charged 175 mL of saturated NaCl wash with agitation. The layers are separated at 30° C. and the bottom aqueous layer is removed, discarding any rag layer with the aqueous layer. The solvent is distilled to a minimum stir volume under vacuum at 80° C. The residual reaction mixture contains (4R,5R)-27.

Step 3. Preparation of 41.

To the reaction mixture of Step 2 is charged 325 mL of methyl ethyl ketone (MEK) and 13 mL water. Next, the reactor is charged 6.2 grams of diazabicyclo[2.2.2]octane (DABCO) dissolved in 25 mL of MEK. The mixture is heated to reflux and held for 30 minutes. Approximately 10% of solvent volume is then distilled off. The mixture starts to form crystals during distillation. The mixture is then cooled to 20° C. for 1 hour. The off-white crystalline 41 (Form II) is filtered and washed with 50 mL MEK, and let dry under vacuum at 100° C.

### EXAMPLE 29B

### Alternate Preparation of (4R,5R)-1-((4-(4-(3,3-dibutyl-7-(dimethylamino)-2,3,4,5-tetrahydro-4-hydroxy-1,1-dioxido-1 -benzithiepin-5-yl)phenoxy)methyl)phenyl)methyl-4aza-1-azoniabicyclo[2.2.2]octane chloride, Form II of 41

A 1000 mL 4 neck jacketed Ace reactor flask is fitted with a mechanical stirrer, a nitrogen inlet, an addition funnel or condenser or distilling head with receiver, a thermocouple, four internal baffles and a Teflon turbine agitator. The flask is purged with nitrogen gas and charged with 25.0 grams of (4R,5R)-28 and 125 mL of N,N-dimethylacetamide (DMAC). The mixture is heated to 50° C. and to it is added 7.11 grams of 30% sodium hydroxide over a period of 15 to 30 minutes with agitation. The mixture is stirred for 30 minutes. To the flask is added 9.5 grams of solid 55. The reactor is stirred for 3 hours. To the mixture is added 1.2 g of solid DABCO. The mixture is stirred 30 to 60 minutes at 50° C. To the flask is added 225 mL of toluene followed by 125 mL of water. The mixture is stirred for 15 minutes and the layers are then allowed to separate at 50° C. The bottom aqueous layer is removed but any rag layer is retained with the organic layer. The flask is then charged with 175 mL of 5% hydrochloric acid solution and stirred 15 minutes. Layers are separated at 50° C. to remove the bottom aqueous layer, discarding any rag layer with the aqueous layer. The flask is then charged with 225 mL of water and stirred 15 minutes. The layers are allowed to separate at 50° C. The bottom aqueous layer is removed, discarding any rag layer with the aqueous layer. Approximately half of the solvent is distilled off under vacuum at a maximum pot temperature of 80° C. The residual reaction mixture contains (4R,5R)-26.

Step 2. Preparation of (4R,5R)-27.

Toluene (112.5 mL) is charged back to the reaction mixture of Step 1 and the mixture is cooled to 25° C. To the mixture is then added 7.3 grams of thionyl chloride over 15 to 45 minutes. The temperature of the mixture is maintained above 20° C. and below 40° C. The reaction turns cloudy on first addition of thionyl chloride, then at about 30 minutes the layers go back together and form a clear mixture. The mixture is then charged with 179.5 mL of 4% NaOH wash over a 30 minute period. The mixture is maintained above 20° C. and below 40° C. during this time. The addition of the wash is stopped when the pH of the mixture reaches 8.0 to 10.0. The mixture is then allowed to separate at 40° C. for at least one hour. The bottom aqueous layer is removed and any rag layer is retained with the organic layer. To the mixture is charged 200 mL of dilution water. The mixture is stirred for 15 minutes and then allowed to separate at 40° C. for at least one hour. The bottom aqueous layer is removed, discarding any rag layer with the aqueous layer. The solvent is distilled to a minimum stir volume under vacuum at 80° C. The residual reaction mixture contains (4R,5R)-27.

Step 3. Preparation of 41.

To the reaction mixture of Step 2 is charged 350 mL of methyl ethyl ketone (MEK) and 7 mL water. The mixture is stirred for 15 minutes and the temperature of the mixture is adjusted to 25° C. Next, the reactor is charged with 6.7 grams of solid diazabicyclo[2.2.2]octane (DABCO). The mixture is maintained at 25° C. for three to four hours. It is then heated to 65° C. and maintained at that temperature for 30 minutes. The mixture is then cooled to 25° C. for 1 hour. The off-white crystalline 41 (Form II) is filtered and washed with 50 mL MEK, and let dry under vacuum at 100° C.

### EXAMPLE 30

### Alternate preparation of (4R,5R)-1-((4-(4-(3,3-dibutyl-7-(dimethylamino)-2,3,4,5-tetrahydro-4-hydroxy-1,1-dioxido-1-benzithiepin-5-yl)phenoxy)methyl)phenyl)methyl-4aza-1-azoniabicyclo[2.2.2]octane chloride, Form I of 41

(4R,5R)-27 (2.82 kg dry basis, 4.7 mol) was dissolved in MTBE (9.4 L). The solution of (4R,5R)-27 was passed through a 0.2 mm filter cartridge into the feeding vessel. The flask and was rinsed with MTBE (2×2.5 L). The obtained solution as passed through the cartridge filter and added to the solution of (4R,5R)-27 in the feeding vessel. DABCO (diazabicyclo[2.2.2]octane, 0.784 kg, 7.0 mol) was dissolved in methanol (14.2 L). The DABCO solution was passed through the filter cartridge into the 100 L nitrogen-flushed reactor. The Pyrex bottle and the cartridge filter were rinsed with methanol (7.5 L) and the solution was added to the reactor. The (4R,5R)-27 solution was added from the feeding vessel into the reactor at 37° C. over a period of 10 minutes, while stirring. Methanol (6.5 L) was added to the Pyrex bottle and via the cartridge filter added to the feeding vessel to rinse the remaining (4R,5R)-27 into the reactor. The reaction mixture was brought to 50-60° C. over 10-20 minutes and stirred at that temperature for about 1 hour. The mixture was cooled to 20-25° C. over a period of 1 hour. To the reaction mixture, methyl t-butyl ether (MTBE) (42 L) was added over a period of 1 hour and stirred for a minimum of 1 hour at 20-25° C. The suspension was filtered through a Büchner funnel. The reactor and the filter cake were washed with MTBE (2×14 L). The solids were dried on a rotary evaporator in a 20 L flask at 400−12 mbar, 40° C., for 22 hours. A white crystalline solid was obtained. The yield of 41 (Form I) was 3.08 kg (2.97 kg dry, 93.8 %) and the purity 99.7 area % (HPLC; Kromasil C 4, 250×4.6 mm column; 0.05% TFA in H₂O/0.05% TFA in ACN gradient, UV detection at 215 nm).

### EXAMPLE 30A

### Conversion of Form I of Compound 41 into Form II of Compound 41

To 10.0 grams of Form I of 41 in a 400 mL jacketed reactor is added 140 mL of MEK. The reactor is stirred (358 rpm) for 10 minutes at 23° C. for 10 minutes and the stirring rate is then changed to 178 rpm. The suspension is heated to reflux over 1 hour using a programmed temperature ramp (0.95° C./minute) using batch temperature control (cascade mode). The delta Tmax is set to 5° C. The mixture is held at reflux for 1 hour. The mixture is cooled to 25° C. After 3 hours at 25° C., a sample of the mixture is collected by filtration. Filtration is rapid (seconds) and the filtrate is clear and colorless. The white solid is dried in a vacuum oven (80° C., 25 in. Hg) to give a white solid. The remainder of the suspension is stirred at 25° C. for 18 hours. The mixture is filtered and the cake starts to shrink as the mother liauor reaches the top of the cake. The filtration is stopped and the reactor is rinsed with 14 mL of MEK. The reactor stirrer speed is increased from 100 to 300 rpm to rinse the reactor. The rinse is added to the filter and the solid is dried with a rapid air flow for 5 minutes. The solid is dried in a vacuum oven at 25 in. Hg for 84 hours to give Form II of 41.

### EXAMPLE 31

### Preparation of 2-(phenylthiomethyl)hexanal

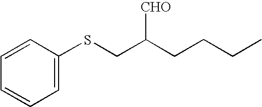

To a stirred mixture of n-butylacrolein (9.5 mL, 71.3 mmol) and Et₃N (0.5 rnL, 3.6 mmol) at 0° C. under nitrogen is added thiophenol (7.3 mL, 71.3 mmol) in 5 minutes. The mixture is allowed to warm to room temperature in 30 minutes. ¹H NMR of the reaction mixture sample will show quantitative conversion. Et₃N is removed under reduced pressure.

### EXAMPLE 32

### Preparation of 2-((4-methoxyphenylthio)methyl)hexanal

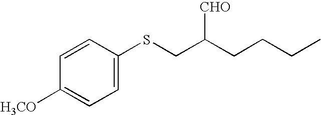

To a stirred mixture of n-butylacrolein (2.66 mL, 20 mmol) and Et₃N (0.14 mL, 1 mmol) at 0C under nitrogen is added 4-methoxythiophenol (2.46 mL, 20 mmol) in 5 minutes. The mixture is allowed to warm to room temperature in 30 minutes. 1HNMR of the reaction mixture sample will show quantitative conversion. Et₃N is then removed under reduced pressure.

### EXAMPLE 33

### Preparation of 2-((4-chlorophenylthio)methyl)hexanal

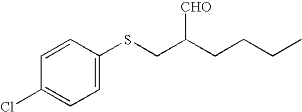

To a stirred mixture of n-butylacrolein (5.32 mL, 40 mmol) and Et₃N (0.28 mL, 2 mmol) at 0° C. under nitrogen is added 4-chlorothiophenol (5.78 g, 40 mmol) in 5 minutes. The mixture is allowed to warm to room temperature in 30 minutes. ¹HNMR of the reaction mixture sample will show quantitative conversion. Et₃N is then removed under reduced pressure.

### EXAMPLE 34

### Preparation of 2-(acetylthiomethyl)hexanal

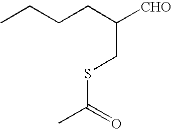

To a stirred mixture of n-butylacrolein (13.3 mL, 100 mmol) and Et₃N (0.7 mL, 5 mmol) at 0° C. under nitrogen is added thioacetic acid (7.2 mL, 100 mmol) in 5 minutes. The mixture is allowed to warm to room temperature in 30 minutes. ¹HNMR of the reaction mixture sample will show quantitative conversion. Et₃N is then removed under reduced pressure.

### EXAMPLE 35

### Preparation of 2-methyl-3-phenylthiopropanal

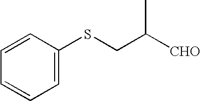

To a stirred mixture of 51.4 g (0.733 mole) of methacrolein and 2 g (0.018 mole) of triethylamine at 0-5° C. is added 80.8 g (0.733 mole) of benzenethiol slowly. The addition rate is such that the temperature was under 10° C. The reaction mixture is stirred at 0-5° C. for one hour. The mixture is placed on a rotary evaporator to remove triethylamine.

### EXAMPLE 36

### Preparation of 2-(((4-chlorophenyl)-sulfonyl)methyl)hexanal

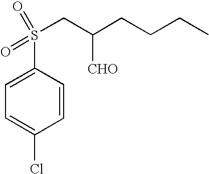

To a stirred solution of 4-chlorobenzosulfinate sodium salt (4.10 g, 20.81 mmol) in 20 mL of acetic acid at 60° C. is added 2-butylacrolein (3.8 mL, 28.56 mmol) slowly.

The reaction mixture us kept at 50° C. for 3.5 hours. The mixture us diluted with 10 mL of water and extracted with ethyl acetate (2×10 mL). The combined extract is washed with saturated NaHCO₃, water, brine, and dried with MgSO₄. After removing solvents, the product is obtained as a yellowish slightly viscous oil in 94% yield.

### EXAMPLE 37

### Preparation of 2-(((4-methylphenyl)sulfonyl)- methyl)hexanal

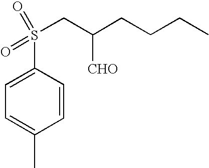

To a stirred solution of 4-toluenesulfinate sodium salt (10.10 g, 56.68 mmol) in 35 mL of acetic acid at 50° C. is added 2-butylacrolein (10.6 mL, 79.66 mmol) slowly. The reaction mixture is kept at 50° C. for 3 hours. After cooling to room temperature, the mixture is diluted with 50 mL of water and extracted with ethyl acetate (2×25 mL). The combined extract is washed with saturated NaHCO₃, water, brine, and dried with MgSO₄. After removing solvents, the product is obtained as a yellow liquid in 75% yield.

### EXAMPLE 38

### Preparation of (4E)-2-(acetylthiomethyl)-2-butylhex4-enal

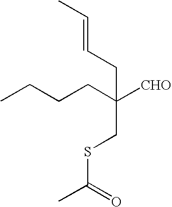

To a stirred solution of 2-(acetylthiomethyl)hexanal (32.6 g, 0.173 mole) in 325 mL of xylenes in a 500-mL RBF fitted with a Dean-Stark trap is added 2-hydroxy-3-butene (22.5 mL, 0.259 mole), followed by pyridinium p-toluenesulfonate (4.34 g, 0.017 mole) at room temperature under nitrogen. The mixture is heated to reflux overnight. After cooling to room temperature, the xylenes solution is washed with 300 mL of saturated NaHCO₃ solution. The aqueous phase is extracted with 300 mL of ethyl acetate. The combined organic extract is washed with 200 mL of brine and 200 mL of water. After removing solvents, the product is obtained by vacuum distillation (157-160° C./1.5 mmHg) in 80.5% yield.

### EXAMPLE 39

### Preparation of (4E)-2-butyl-2-(phenylthiomethyl)hex4-enal

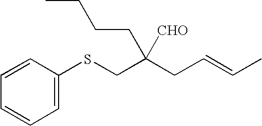

2-(Phenylthiomethyl)hexanal (2.67 g, 12 mmol), 3-buten-2-ol (5 mL, 58 mmol), and p-toluenesulfonic acid (0.05 g, 0.26 mmol) are added to 25 mL of xylenes. The reaction mixture is heated to reflux using a Dean-Stark trap to collect water. After 3 hours, the mixture is cooled to room temperature and diluted with ethyl acetate, which is washed saturated NaHCO₃ solution, brine, and dried with MgSO₄. After removing solvents, the crude product is purified by chromatography. The product is obtained in 78.6% as a colorless oil.

### EXAMPLE 40

### Preparation of (4E)-2-methyl-2-(phenylthiomethyl)-hept4-enal

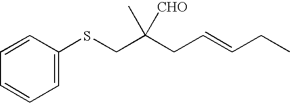

2-Methyl-3-phenylthiopropanal (9.07 g, 0.05 mole), 1-penten-3-ol (21.67 g, 0.25 mole), and p-toluenesulfonic acid (0.24 g, 0.0013 mole) are added to 90 mL of xylenes. The reaction mixture is heated to reflux using a Dean-Stark trap to collect water. After 3 hours, the mixture is cooled to room temperature and quenched with 30 mL of saturated NaHCO₃ solution. The two phases are separated and the aqueous phase is extracted with 30 mL of ethyl acetate. The combined organic extracts is washed with 30 mL of brine and dried with Na₂SO₄. After removing solvents, the crude product is purified by chromatography. The product is obtained in 77% as a colorless oil.

### EXAMPLE 41

### Preparation of (4E)-2-methyl-2-(phenylthiomethyl)-hex-4-enal

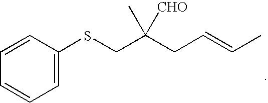

2-Methyl-3-phenylthiopropanal (9.07 g, 0.05 mole), 3-buten-2-ol (18.04 g, 0.25 mole), and p-toluenesulfonic acid (0.24 g, 0.0013 mole) are added to 90 mL of xylenes. The reaction mixture is heated to reflux using a Dean-Stark trap to collect water. After 3 hours, the mixture is cooled to room temperature and quenched with 30 mL of saturated NaHCO₃ solution. The two phases are separated and the aqueous phase is extracted with 30 mL of ethyl acetate. The combined organic extracts is washed with 20 mL of brine and dried with Na₂SO₄. After removing solvents, the crude product is purified by chromatography. The product is obtained in 74.3% as a colorless oil.

### EXAMPLE 42

### Preparation of (4E)-2-butyl-2-(((4-chlorophenyl)-sulfonyl)methyl)hex4-enal

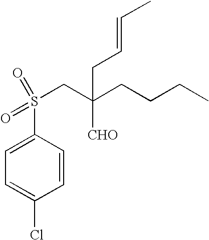

To a stirred solution of 2-(((4-chlorophenyl)-sulfonyl)methyl)hexanal (3.38 g, 11.73 mmol) in 30 mL of toluene in a RBF fitted with a Dean-Stark trap is added 2-hydroxy-3-butene (5 mL, 57.73 mmol), followed by p-toluenesulfonic acid (0.13 g) at room temperature under nitrogen. The mixture is heated to reflux for 20 hours. After cooling to room temperature, the toluene solution is diluted with 10 mL of ethyl acetate and washed with 10 mL of saturated NaHCO₃ solution. The aqueous phase is extracted with ethyl acetate. The combined organic extract is washed with water (2×10 mL), brine (1×10 mL), and dried with MgSO₄. After removing solvents, the product is obtained as a brownish oil in 98% yield.

### EXAMPLE 43

### Preparation of (4E)-2-butyl-2-(((4-methylphenyl)-sulfonyl)methyl)hex-4-enal

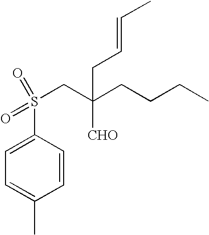

To a stirred solution of 2-(((4-methylphenyl)-sulfonyl)methyl)hexanal (5.63 g, 21 mmol) in 35 mL of toluene in a RBF fitted with a Dean-Stark trap is added 2-hydroxy-3-butene (10 mL, 115 mmol), followed by p-toluenesulfonic acid (0.13 g) at room temperature under nitrogen. The mixture is heated to reflux overnight. After cooling to room temperature, the toluene solution is washed with saturated NaHCO₃ solution (2×10 mL), water (2×20 mL), brine (1×20 mL), and dried with MgSO₄. After removing solvents, the product is obtained as a brownish oil in quantitative yield with a GC purity of 89%.

### EXAMPLE 44

### Preparation of 2-butyl-2-(((4-methylphenyl)-sulfonyl)methyl)hexanal

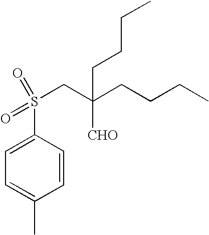

To a solution of 0.5 g of 2-butyl-2-(((4-ethyl-phenyl)sulfonyl )methyl)hexanal in 30 mL of toluene is added 5 mL of 37% formaldehyde and 220 mg of 20% Pd(OH)₂/C catalyst. The reaction mixture is purged with dry nitrogen gas (3×) and hydrogen gas (3×) and hydrogenated at 60 psi H2 and 60° C. for 15 hours. The catalyst is removed by filtration and washed with ethanol (2×20 mL). Solvents of the combined washes and filtrate are removed under vacuum to yield the crude product.

For the following examples ¹H and ¹³C NMR spectra were recorded on a Varian 300 spectrometer at 300 and 75 MHz respectively. The ¹H chemical shifts are reported in ppm downfield from tetramethylsilane. The ¹³C chemical shifts are reported in ppm relative to the center line of CDCl₃ (77.0 ppm). Melting points were recorded on a Buchi 510 melting point apparatus and are uncorrected. HPLC data was obtained on a Spectra Physics 8800 Chromatograph using a Beckman Ultrasphere C18 250×4.6 mm column. HPLC conditions: detector wavelength=254 nm, sample size=10 μL, flowrate=1.0 mL/minute, mobile phase=(A) 0.1% aqueous trifluoroacetic acid: (B) acetonitrile. Quantitative HPLC analysis was determined by running samples of known concentration of the crude product and of purified product, adjusting the peak areas for concentration differences, and dividing the peak area of the crude sample by the peak area of the purified sample.

HPLC Gradient:

| Time | % A | % B |
|---|---|---|
| 0 min | 50 | 50 |
| 5 min | 50 | 50 |
| 30 min | 0 | 100 |
| 40 min | 0 | 100 |

### EXAMPLE 45

### Preparation of compound 32

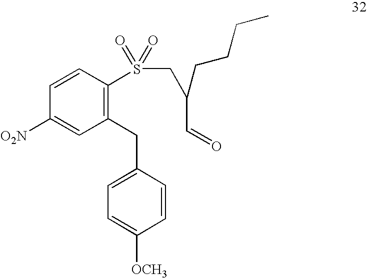

Procedure A:

Na₂S.9H₂O (8.64 g, 36.0 mmol) and sulfur (1.16 g, 36.0 mmol) were combined in a 50 mL round-bottom flask. The mixture was heated to 50° C. until homogeneous, and water (10.0 rnL) was added. Compound 33 (10.00 g, 36.0 mmol) and S ethanol (100 mL) were combined in a 500 mL round-bottom flask. The reaction flask was purged with N₂ and equipped with mechanical stirrer. The reaction mixture was heated to 65° C. until homogeneous, and then increased to 74° C. The disulfide solution was added to the 500 mL reaction flask over 10 minutes. After 1.5 hours at reflux, analysis of an aliquot by HPLC indicated complete conversion of 33. Aqueous 18% NAOH (20.0 g, 90.0 mmol) was added over 5 minutes (endothermic). After 15 minutes, the reaction mixture was cooled to 0C, and 30% H₂O₂ (16.00 g, 140.0 mmol) was added dropwise keeping temp below 20° C. After 1.5 hours at <20° C., analysis of an aliquot by HPLC indicated total oxidation of the sodium thiophenolate intermediate. The ethanol was removed under reduced pressure at <65° C. Water (100 mL) was added, and the mixture was washed with CH₂Cl₂ (100 mL). 10% HCl (˜40 mL) was added until pH =1, and the reaction mixture was extracted with CH₂Cl₂ (100.0 mL). 2-Butylacrolein (5.20 mL, 39.2 mmol) was added to the organic extract, and the mixture was stirred for 1 hour. Analysis of an aliquot by HPLC indicated very little sulfinic acid intermediate. The organic layer was concentrated in vacuo to give an amber solid (14.19 g). Analysis by quantitative HPLC indicated 84% purity, which corresponds to 11.92 g Michael adduct (79% yield of 32 based on 33.

Procedure B:

Compound 33 (4.994 g, 17.98 mmol) and dimethylacetamide (21.0 mL) were combined in a dry 250 mL round-bottom flask. The reaction flask was purged with N₂, equipped with magnetic stirrer, and heated to 40° C. until the mixture became homogeneous. Na₂S.3H₂O (2.91 g, 22.37 mmol) and water (4.0 mL) were combined in a separate flask and heated to 55° C. until homogeneous. The Na₂S solution was then added portion-wise to the reaction flask over 25 minutes. After 2.5 hours at 40° C., analysis of an aliquot by HPLC indicated complete conversion of 33. After 2 hours more, the reaction mixture was cooled to 30° C., and aqueous 18% NaOH (10.02 g, 44.90 mmol) was added. After 20 minutes, the reaction mixture was cooled to 0° C., and 30% H₂O₂ (8.02 g, 70.6 mmol) was added dropwise over 30 minutes while maintaining a temperature of less than 15° C. After 10 minutes, an aliquot was removed and analyzed by HPLC, which indicated >93% oxidation of the sodium thiophenolate intermediate. After 1 hour, Na₂SO₃ (6.05 g, 48.0 mmol) and water (50.0 mL) were added, and the cooling bath was removed. After 20 minutes, the mixture was washed with toluene (or CH₂Cl₂) (2×50.0 mL). Toluene (or CH₂Cl₂) (50.0 mL), 2-butylacrolein (2.60 mL, 19.6 mmol), and n-Bu₄NI (0.032 g, 0.087 mmol) were added, and the reaction mixture was cooled to 0° C. To this, 10% HCl (˜30 mL) was added until pH=1. The cooling bath was removed, and the reaction mixture was stirred for 30 minutes. Analysis of an aliquot of the aqueous layer by HPLC indicated very little sulfinic acid intermediate. After 30 minutes more, the aqueous layer was separated and discarded. The organic layer was kept at −10° C. overnight, stirred at room temperature for 5 hours. Analysis of the toluene solution by quantitative HPLC indicated 6.444 g Michael adduct, (85% yield of 32 based on 33).

For characterization, a portion of the crude product was concentrated in vacuo and precipitated from ethyl ether to afford a yellow solid: mp 62.0-76.0° C.; HPLC (CH₃CN/H₂O): rt=22.4 minutes. ¹H NMR (CDCl₃) □□□t, J=6.0 Hz, 3H), 1.24 (m, 4H), 1.53 (m, 1H), 1.70 (m, 1H), 2.83 (dd, J=14.1, 4.2 Hz, 1H), 2.98 (m, 1H), 3.56 (dd, J=(4.4, 7.3 Hz, 1H), 3.79 (s, 3H), 4.53, s, 2H), 6.87 (dd, J=.6, 2.4 Hz, 2H), 7.13 (d, J=8.7 Hz, 2H), 8.12 (s, 1H), 8.20 (d, J=1.2 Hz, 2H), 9.53 (d, J=0.9 Hz, 1H). ¹³C NMR (CDCl₃) □13.6, 22.4, 28.1, 28.5, 37.4, 45.4, 53.9, 55.2, 114.4, 121.7, 127.3, 129.6, 130.3, 132.1, 142.7, 144.1, 150.7, 158.7, 199.5. HRMS (ES+) calcd for C₂₁H₂₅NO₆S+NH₄: 437.1731, found: 437.1746. Anal. (C₂₁H₂₅NO₆S): C, 60.13; H, 6.01; N, 3.34; O, 22.88; S, 7.64. Found: C, 60.22; H, 5.98; N, 3.32; O, 22.77; S, 7.73.

### EXAMPLE 46

### Preparation of Compound 18a

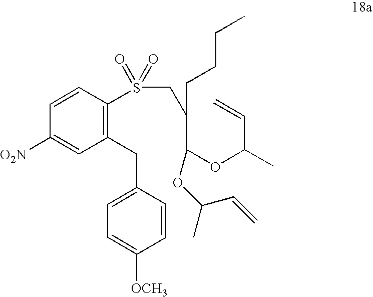

Procedure A:

Compound 32 (11.577 g, 27.598 mmol), p-toluenesulfonic acid monohydrate (0.6115 g, 3.21mmol), CH₂Cl₂ (70nL) and 3-buten-2-ol (13.91 mL, 160.5mmol) were combined in a dry 250 mL round-bottom flask. The reaction flask was purged with N₂ and equipped with magnetic stirrer, Dean Stark trap, and reflux condenser. The reaction mixture was heated to reflux. After 10.25 hours, analysis of an aliquot by HPLC indicated 78.6% 18a, 13.3% pre-Claisen enol ether, 3.7% 32 and approximately 4% byproducts. K₂CO₃ (1.50 g, 10.8 mmol) was added to the reaction flask. After 2.5 hours, CH₂Cl₂ (50.0 mL) was added, and the mixture was filtered (15.73 g). Quantitative HPLC was performed using a sample of purified 18a. The total peak area of the crude product was determined by summing the pre-Claisen enol ether and 18a peaks. It was assumed that they have the same HPLC response factors. Analysis by quantitative HPLC indicated 90% purity, which corresponds to 14.20g 18a and pre-Claisen enol ether 47, (94% yield of 18a based on 22).

Procedure B:

Compound 32 (5.43 g, 12.9 mmol), 3-buten-2-ol (76.16 g, 85.4 mmol), p-toluenesulfonic acid monohydrate (0.258 g, 1.36 mmol) and toluene (51.0 mL) were combined in a 100 mL round-bottom flask. The reaction flask was purged with N₂ and equipped with magnetic stirrer, Dean Stark trap, condenser, and vacuum line. The condenser was cooled to −10° C. via a Cryocool bath, and the Dean Stark trap was filled with 3-buten-2-ol (about 11 mL). The reaction flask was evacuated to 107.5 mmHg via a pressure controller and heated to 49° C. After 4 hours, the reaction flask was cooled to room temperature and concentrated in vacuo at 30° C. The crude product was collected as an amber oil (8.154g). Quantitative HPLC was performed using a sample of purified 18a. The total peak area of the crude product was determined by summing the pre-Claisen enol ether and 18a peaks. It was assumed that they have the same HPLC response factors. Analysis by quantitative HPLC indicated 69% purity, which corresponds to 5.626g 18a and pre-Claisen enol ether 47, (80% yield of 18a based on 32)):

HPLC (CH₃CN/H₂O): 18a: rt=32.56, 32.99, 33.09 minutes, pre-Claisen enol ether: rt=30.7 minutes. ¹H NMR (CDCl₃) □0.84-0.93 (m, 3H), 1.09-1.34 (m, 10H), 1.40-1.70 (m, 2H), 2.16-2.35 (m, 1H), 2.88-2.98 (m, 1H), 3.52-3.63 (m, 1H), 3.80 (m, 3H), 3.84-4.10 (m, 2H), 4.49 (s, 1H), 4.50 (s, 1H), 4.59 (d, J=3.0 Hz, 0.25H), 4.60 (d, J=2.7 Hz, 0.25H), 4.65 (d, J=2.4 Hz, 0.25H), 4.70 (d, J=2.4 Hz, 0.25H), 5.00-5.18 (m, 4H), 5.42-5.84 (m, 2H), 6.87 (d, J=8.7 Hz, 1H), 6.88 (d, J=8.4 Hz, 1H), 7.12-7.17 (m, 2H), 8.02 (t, J=2.4 Hz, 1H ), 8.14-8.17 (m, 1H), 8.23-8.27 (m, 1H); ¹³C NMR (CDCl₃) □13.8, 20.1, 20.9, 21.0, 21.4, 21.51, 21.57, 21.6, 22.53, 22.55, 22.57, 28.7, 28.8, 28.94, 28.99, 29.0, 29.3, 29.4, 29.8, 37.1, 37.2, 37.3, 38.73, 38.75, 53.3, 55.2, 55.60, 55.66, 55.7, 55.9, 73.4, 73.5, 73.8, 73.9, 74.3, 75.1, 75.9, 97.7, 98.3, 98.4, 99.5, 113.6, 114.4, 114.5, 114.9, 115.7, 115.9, 116.1, 116.3, 116.7, 116.9, 121.22, 121.26, 121.31, 121.34, 126.70, 126.75, 126.8, 129.73, 129.77, 130.45, 130.48. 130.5, 131.51, 131.51, 131.57, 139.6, 139.8, 139.9, 140.1, 140.2, 140.3, 143.6, 143.70, 143.71, 143.81, 143.84, 144.26, 144.29, 144.34, 144.35, 144.37, 150.5, 158.6; HRMS (ES+) calcd for C₂₉H₃₉NO₇S +NH₄: 563.2791, found: 563.2804.

### EXAMPLE 47

### Preparation of compound 31

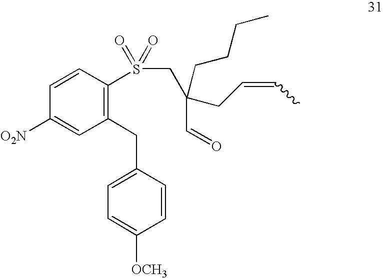

Procedure A:

A crude mixture of 18a and pre-Claisen enol ether 47 (13.636 g, 24.989 mmol), o-xylene (75.0 mL), and calcium hydride (0.334 g, 7.93 mmol) were combined in a dry 250 mL round-bottom flask. The reaction flask was purged with N₂, equipped with magnetic stirrer, and heated to 145° C. After 3 hours, an aliquot was removed and analyzed by HPLC, which indicated 93% 31, 1% 32, 3% pre-Claisen enol ether 47, and 4% byproducts. The reaction mixture was cooled to RT and filtered through celite washing with o-xylene (50.0 mL). The crude product was concentrated in vacuo and collected as an amber oil (11.525g). Analysis by quantitative HPLC indicated 86% purity, which corresponds to 9.9115 g Claisen product (80% yield based on the mixture of 31and pre-Claisen enol ether 47).

Procedure B:

A crude mixture of 18a and pre-Claisen enol ether 47(2,700 g, 4.948 mmol), toluene (15.0 mL) and calcium hydride (0.0704 g, 1.67 mmol) were combined in a dry Fischer-Porter bottle. The reaction flask was purged with N₂, equipped with magnetic stirrer, and heated to 145° C. After 10 hours, analysis of an aliquot by HPLC indicated 90.9% Claisen product 31), 2.8% pre-Claisen enol ether 47, 1.3% 18a and 5% byproducts. Toluene (30.0 mL) was then added, and the mixture was filtered through celite. Concentration in vacuo of the filtrate afforded the crude product as an amber oil (2.6563 g). Analysis by quantitative HPLC indicated 82% purity, which corresponds to 2.1782 g Claisen product 31, (93% yield based on the mixture of 18a and pre-Claisen enol ether 47).

Procedure C:

Purified 18a (0.228 g, 0.417 mmol) was placed in a 100 mL round-bottom flask. The reaction flask was placed in a Kugelrohr apparatus and evacuated to 100 mtorr. After 1 hour, the apparatus was heated to 40° C. After 15 minutes more, the apparatus was heated to 145° C. After 1 hour, the apparatus was cooled to room temperature to afford an dark oil (0.171 g). Analysis by HPLC indicated 88% Claisen product 31, 3% pre-Claisen enol ether 47, 3% 18a and 6% byproducts. This corresponds to an 81% yield based on 18a. Quantitative HPLC was not performed.

For characterization, a portion of the residue was purified by flash column chromatography on silica gel (eluting with EtOAc/hexanes), concentrated in vacuo, and the desired product was collected as an amber oil: HPLC(CH₃CN/H₂O): rt=29.1 minutes. ¹H NMR (CDCl₃) □0.88 (t, J=6.9Hz, 3H), 1.06 (m, 1H), 1.17-1.34 (m, 3H), 1.61 (d, J=6.3 Hz, 3H), 1.68 (m, 1H), 1.83-1.93 (m, 1H), 2.42 (dd, J=14.4, 6.6 Hz, 1H), 2.63 (dd, J=14.7, 8.1 Hz, 1H), 3.12 (s, 2H), 3.80 (s, 3H), 4.52 (ABq, 2H), 5.16-5.26 (m, 1H), 5.52-5.64 (m, 1H), 6.88 (d, J=8.4 Hz, 2H), 7.11 (d, J=8.7 Hz, 2H), 8.09 (s, 1H), 8.21 (s, 1H), 8.22 (s, 1H), 9.40 (s, 1H)□ ¹³C NMR (CDCl₃) □13.7, 17.9, 22.8, 25.6, 32.6, 35.9, 37.2, 52.6, 55.1, 57.2, 114.4, 121.7, 123.4, 127.1, 129.8, 130.2, 131.2, 131.5, 143.7, 144.5, 150.5, 158.7, 202.5. HRMS (ES+) calcd for C₂₅H₃₁NO₆S+NH₄: 491.2216. found: 491.2192. Anal. (C₂₅H₃₁NO₆S): C, 63.40; H, 6.60; N, 2.96; O, 20.27; S, 6.77. Found: C, 63.36; H, 6.39; N, 3.05; O, 20.59; S, 6.71.

Other reactions to form Claisen product 31

General procedure for other reactions of acetal to: In a typical reaction, the purified acetal 18a is combined with solvent, base and water removing agent (if indicated) and heated. The zeolites and molecular sieves are activated at 300° C. The reported conversion is based on the peak area of 31 vs. 18a in the HPLC data. The reported yield is based on the peak area of the products vs. byproducts in the HPLC data. The results are summarized below.

| Example No. | Base/Conditions | Results |
|---|---|---|
| 48 | 100° C. | 95% conv./32% yield @ 4 hrs. |
| 49 | 4 A sieves/o-xylene/145° C. | 6% conv./39% yield @ 5 hrs. |
| 50 | o-xylene/120° C. | 100% conv./58% yield @ 2.5 |
| 51 | o-xylene/145° C. | 100% conv./70% yield @ 2 hrs. |
| 52 | CH₃CN/140° C. | 0% conv. @ 6 hrs. |
| 53 | PPTS(0.1 eq.)/pyr.(0.15 eq.)/o- | 84% conv./74% yield @ 3 hrs. |
| | xylene/120° C. | |
| 54 | PPTS(0.13 eq.)/4 A sieves/o- | 21% conv./74% yield @ 1 hrs. |
| | xylene/120° C. | |
| 55 | pyr.(9.0 eq.)/CH₃CN/140° C. | 0% conv. @ 2.5 hrs. |
| 56 | pyr.(12.3 eq.)/xylenes/140° C. | 1% conv./100% yield @ 2 hrs. |
| 57 | Et₃N(0.3 eq.)/o-xylene/145° C. | 19% conv./78% yield @ 6 hrs. |
| 58 | CaH₂(0.46 eq.)/4 A sieves/o- | 97% conv./92% yield @ 5 hrs. |
| | xylene/145° C. | |
| 59 | CaH₂(0.3 eq.)/PhCH₃/145° C. | 96% conv./95% yield @ 10 hrs. |
| 60 | CaH₂(0.43 eq.)/PTSA(0.07 eq.)/4 A | 100% conv./34% yield @ 1 hrs. |
| | sieves/o-xylene/145° C. | |
| 61 | CaH₂(0.42 eq.)/4 A | 0.2% conv./11% yield @ 8 hrs. |
| | sieves/CH₂Cl₂/145° C. | |
| 62 | PhCH₃/prefilter through basic | 98% conv./79% yield @ 3.5 hrs. |
| | alumina/145° C. | |
| 63 | AlCl₃(2.0 eq.)/Et₃N(4.1 | 0% conv. @ 4 hrs. |
| | eq.)/THF/25° C. | |
| 64 | Pd(PhCN)₂Cl₂ (0.1 eq.)/THF/25° C. | reversion to 32. |
| 65 | BF₃ .OEt₂(1.2 eq.)/CH₂Cl₂/-50° C. | reversion to 32. |
| 66 | HMDS/TMSI/CH₂Cl₂/25° C. | 0% conv. @ 5 hrs. |

Other rections to form acetal 18a and the Pre-Claisen enol Ether 47

General Procedure:

In a typical reaction, the sulfone aldehyde 32 is combined with 3-buten-2-ol (about 5 to about 50 eq.), solvent and acid source indicated. If indicated, 4 A molecular sieves (50 wt %), and trimethyl orthoformate TMOF (1.2 eq.) are added to the reaction flask. If no solvent is indicated, 3-buten-2-ol is the solvent. The zeolites and molecular sieves are activated at 300° C. The observed products are a mixture of the acetal 18a and the pre-Claisen enol ether, as determined by LCMS and NMR. The reported conversion is based on the peak area of product(s) vs. 32 in the HPLC data. The reported yield is based on the peak area of the products vs. byproducts in the HPLC data. The results are summarized below.

| Example | | |
|---|---|---|
| No. | Acid/Conditions | Results |
| 67 | TFA(0.24 eq.)/CH₃CN/4 Å | 2.5% conv./50% yield @ 18 hrs. |
| | sieves/25° C. | |
| 68 | TFA(3.5 eq.)/4 Å sieves/50° C. | 42% conv./74% yield @ 4.5 hrs. |
| 69 | TFA(3.8 eq.)/Isopropenyl acetate(3.3 | 44% conv./95% yield @ |
| | eq.)/50° C. | 2 hrs. |
| 70 | TFA(3.5 eq.)/65° C. | 68% conv./86% yield @ 5.5 hrs. |
| 71 | TFA(3.0 eq.)/90° C. | 73% conv./75% yield @ 5.5 hrs. |
| 72 | TFA(3.0 eq.)/PhCH₃/4 Å | 90% conv./53% yield @ 58 hrs. |
| | sieves/TMOF/120° C. | |
| 73 | TFA(3.0 eq.)/CH₃CN/4 Å | 92% conv./58% yield @ 41 hrs. |
| | sieves/TMOF/120° C. | |
| 74 | PTSA(0.1 eq.)/25° C. | 78% conv./100% yield @ 16 hrs. |
| 75 | PTSA(0.1 eq.)/4 Å sieves/50° C. | 87% conv./99% yield @ |
| | | 2 hrs. |
| 76 | PTSA(0.1 eq.)/4 Å sieves/70° C. | 95% conv./92% yield @ 5.75 hrs. |
| 77 | PTSA(0.1 eq.)/4 Å sieves/90° C. | 87% conv./74% yield @ |
| | | 2 hrs. |
| 78 | PTSA(0.1 eq.)/Isopropenyl acetate (3.3 | 63% conv./94% yield @ 2.5 hrs. |
| | eq.)/50° C. | |
| 79 | PTSA(0.12 eq.)/Isopropenyl acetate | 83% conv./91% yield @ |
| | (3.2 eq.)/90° C. | 1 hrs. |
| 80 | PTSA(0.1 eq.)/PhCH₃/4 Å | 29% conv./70% yield @ 18 hrs. |
| | sieves/TMOF/90° C. | |
| 81 | PTSA(0.3 eq.)/PhCH₃/4 Å | 37% conv./70% yield @ 70 hrs. |
| | sieves/TMOF/120° C. | |
| 82 | PTSA(0.1 eq.)/PhCH₃/49° C. @ | 95% conv./93% yield @ 3.5 hrs. |
| | 107.5 mmHg | |
| 83 | PTSA(0.1 eq.)/o-xylene/4 Å | 92% conv./96% yield @ 3.5 hrs. |
| | sieves/50° C. | |
| 84 | PTSA(0.1 eq.)/o-xylene/50° C. | 59% conv./58% yield @ 7.5 hrs. |
| 85 | PTSA(0.1 eq.)/CH₂Cl₂/4 Å | 95% conv./100% yield @ 3.5 hrs. |
| | sieves/47° C. | |
| 86 | PTSA(0.05 eq.)/CH₂Cl₂/4 Å | 95% conv./99% yield @ |
| | sieves/47° C. | 5 hrs. |
| 87 | PTSA(0.025 eq.)/CH₂Cl₂/4 Å | 15% conv./91% yield @ 6.5 hrs. |
| | sieves/47° C. | |
| 88 | PTSA(0.1 eq.)/CH₂Cl₂/47° C. | 100% conv./96% yield @ 1 hrs. |
| 89 | PTSA(0.1 eq.)/EtOAc/90° C. | 75% conv./85% yield @ |
| | | 5 hrs. |
| 90 | PTSA(0.1 eq.)/EtOAc/4 Å sieves/50° C. | 44% conv./85% yield @ |
| | | 1.5 hrs. |
| 91 | PTSA(0.1 eq.)/iPrOAc/4 Å sieves/50° C. | 62% conv./93% yield @ |
| | | 6 hrs. |
| 92 | PTSA(0.1 eq.)/BuOAc/4 Å sieves/50° C. | 72% conv./69% yield @ |
| | | 6 hrs. |
| 93 | PTSA(0.1 eq.)/THF/4 Å | 63% conv./94% yield @ |
| | sieves/50° C. | 7 hrs. |
| 94 | PTSA(0.24 eq.)/CH₃CN/4 Å sieves/25° C. | 85% conv./100% yield @ |
| | | 19 hrs. |
| 95 | PTSA(0.1 eq.)/MIBK/4 Å sieves/50° C. | 59% conv./95% yield @ |
| | | 3 hrs. |
| 96 | PTSA(0.1 eq.)/PhCF₃/50° C. | 55% conv./65% yield @ |
| | | 4 hrs. |
| 97 | PTSA(0.15 eq.)/Pd(PhCN)₂Cl₂ | 100% conv./97% yield @ |
| | (0.09 eq.)/4 Å sieves/25° C. | 23 hrs. |
| 98 | PPTS(0.$$ eq.)/4 Å sieves/ | 65% conv./87% yield @ |
| | 90° C. | 7.5 hrs. |
| 99 | CBV 5020 zeolites(25 wt %)/CH₃CN/25 | 30% conv./97% yield @ |
| | | 22 hrs. |
| 100 | CBV 5020 zeolites(25 wt %)/ | 81% conv./99% yield @ |
| | 4 Å sieves/50° C. | 2 hrs. |
| 101 | CBV 5020 zeolites(25 wt %)/ | 66% conv./94% yield @ |
| | 4 Å sieves/70° C. | 24 hrs. |
| 102 | CBV 5020 zeolites(25 wt %)/ | 81% conv./98% yield @ |
| | 4 Å sieves/90° C. | 1 hrs. |
| 103 | CBV 5020 zeolites(25 wt %)/ | 71% conv./93% yield @ |
| | 90° C. | 2 hrs. |
| 104 | CBV 5020 zeolites(25 wt %)/Isopropenyl | 79% conv./91% yield @ |
| | acetate | 1.5 hrs. |
| | (3.0 eq.)/90° C. | |
| 105 | CBV 5020 zeolites(10 wt %)/PhCH₃/4 Å | 40% conv./53% yield @ |
| | sieves/TMOF/ | 21 hrs. |
| | 120° C. | |
| 106 | 300WN0030g zeolites(10 wt %)/PhCH₃$$ | 22% conv./57% yield @ |
| | sieves/ | 21 hrs. |
| | TMOF/120° C. | |
| 107 | Montmorillonite K10(10 wt. %)/PhCH₃/4 | 70% conv./64% yield @ |
| | sieves/TMOF/120° C. | 57 hrs. |
| 108 | Montmorillonite K10(20 wt %)/ | 4% conv./99% yield @ |
| | 4 Å sieves/25° C. | 18 hrs. |
| 109 | Montmorillonite K10(20 wt %)/CH₃CN/4 | 4% conv./99% yield @ |
| | sieves/25° C. | 21 hrs. |
| 110 | Amberlyst 15(20 wt. %)/ | 49% conv./96% yield @ |
| | CH₂Cl₂/4 Å sieves/47° C. | 2 hrs. |
| 111 | Acetic acid(0.24 eq.)/ | 0% conv./0% yield @ |
| | CH₃CN/4 Å sieves/25° C. | 22 hrs. |
| 112 | Acetic acid(3.0 eq.)/90° C. | 15% conv./78% yield @ |
| | | 2.5 hrs. |
| 113 | Acetic acid (3.0 eq.)/4 Å sieves/90° C. | 79% conv./84% yield @ |
| | | 6.5 hrs. |
| 114 | HCl (0.20 eq.)/25° C. | 3% conv./6% yield @ |
| | | 1 hrs. |
| 115 | HCl (4.1 eq.)/4 Å sieves/ | 87% conv./98% yield @ |
| | 25° C. | 2.5 hrs. |
| 116 | HCl (1.1 eq.)/dioxane/4 Å sieves/25° C. | 67% conv./100% yield @ |
| | | 1 hrs. |
| 117 | HCl (1.1 eq.)/CH₂Cl₂/4 Å sieves/47° C. | 69% conv./100% yield @ |
| | | 1 hrs. |
| 118 | AlClEt₂/0.16 eq.)/4 Å sieves/25° C. | 80% conv./59% yield @ |
| | | 47 hrs. |
| 119 | Pd(PPh₃)₄ (0.10 eq.)/4 Å sieves/25° C. | retro-Michael reaction |
| | | only |
| 120 | Pd(PhCN)₂Cl₂ (0.10 eq.)/ | 5% conv./47% yield @ |
| | THF/4 Å sieves/25° C. | 4.5 hrs. |
| 121 | Pd(PhCN)₂Cl₂ (0.12 eq.)/ | 63% conv./100% yield @ |
| | 4 Å sieves/25° C. | 2 hrs. |

### EXAMPLE 122

### Preparation of Compound 29.

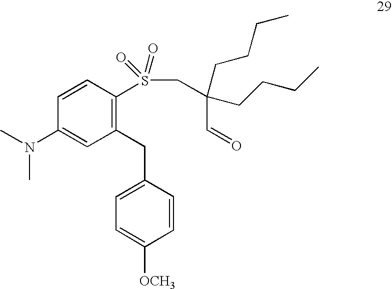

To a solution of 0.434 g of compound 31 in 30 mL of hot ethanol was added 5 mL of 37% formaldehyde and 220 mg of 20% Pd(OH)₂/C catalyst. The reaction mixture was purged with nitrogen gas (3×) and H₂ (3×) and hydrogenated at 60 psi and 60° C. for 15 hours. The catalyst was removed by filtration and washed with ethanol (2×20 mL). Solvents of the combined washes and filtrate were removed to yield 370 mg of crude 29 (85%). An analytical sample was obtained by recrystallization from ethanol and water.

### EXAMPLE 123

### Preparation Compound 12c

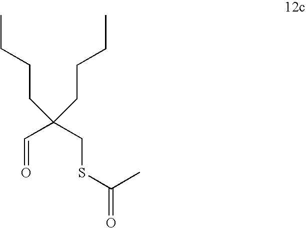

A 1 L 3-neck jacked flask is fitted with baffles, a bottom valve, an overhead stirred, an addition funnel, and a Neslab cooling bath. To the reactor is charged 35 grams of potassium thioacetate. The reactor is flushed with nitrogen gas and to it is charged 85 mL of dimethylformamide (D)M. Mixing is started at 180 rpm and the bath is cooled to 18° C. The reactor is again flushed with nitrogen gas and to it is added 73.9 grams of compound 53 over 20 minutes via a dropping funnel. The pot temperature is maintained at 23° C. during the addition. The mixture is stirred for 1 hour at about 23° C. to 27° C. To the mixture is then added 80 mL of water followed by 100 mL of ethyl acetate. The mixture is stirred for 20 minutes. The layers are allowed to separate and the aqueous layer is drained off. To the pot is added another 50 mL of water and the mixture is stirred for 15 minutes. The layers are separated and the aqueous layer is drained off. Then to the pot is added 50 mL of brine and the mixture is stirred for another 15 minutes. The layers are separated and the aqueous layer is removed. The organic layer is concentrated under reduced pressure (water aspirator pressure) at 47° C. to obtain 68.0 grams of orange oily compound 12c.

### EXAMPLE 124

### Preparation of Diethyl Acetal Compound 12d

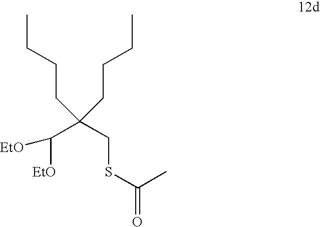

A 250 mL 3-neck round bottom flask is fitted with an overhead stirrer, a Teflon coated temperature probe, and a separatory funnel. To the flask is charged 78 g of compound 12c and 200 mL of ethanol. The reactor is flushed with nitrogen gas and to it is charged 60 mL of triethylorthoformate. Then to the flask is added 4 grams of p-toluenesulfonic acid. The mixture is stirred at room temperature for 16 hours. The mixture is then concentrated under reduced pressure and to the flask is added 100 mL of ethyl acetate. Next is added 1.7 grams of sodium bicarbonate in 50 mL of water. The mixture is stirred for 3 minutes. The layers are allowed to separate and the aqueous layer is drained. The organic layer is filtered through a pad of sodium sulfate and the organic layer is concentrated under reduced pressure (water aspirator pressure) to afford 96.42 grams of orange oily compound 12d.

### EXAMPLE 125

### Preparation of Diethyl Acetal Compound 67.

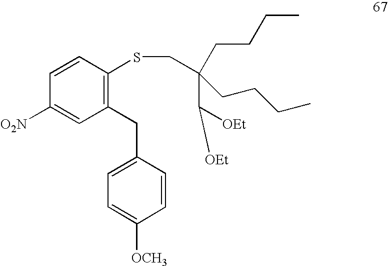

A 0.5 L 3-neck jacked flask is fitted with baffles, a bottom valve, an overhead stirrer, an addition funnel, a nitrogen inlet, a silicon oil bubbler, a Teflon-coated temperature probe, and a PolyScience cooling/heating bath. To the flask is charged 48.85 grams of compound 33. The flask is flushed with nitrogen gas and to it is charged 75 mL of DMSO. The mixture is again flushed with nitrogen and agitation is begun. The jacket temperature is set at 40° C. and to the flask is added 56.13 grams of compound 12d. Stirring is continued for 30 minutes and to the mixture is slowly added 28 mL of 50% aqueous NaOH over 120 minutes via a dropping funnel. The mixture is stirred for 3 hours while maintaining the jacket temperature at 40° C. The reaction is allowed to cool to ambient temperature and the mixture is stirred for 15 hours (overnight). The jacket temperature is then adjusted to 5° C. and to the mixture is slowly added 300 mL of water. The reaction is exothermic. The biphasic mixture is transferred to a separatory funnel and the mixture is extracted with 2×150 mL of ethyl acetate. The layers were allowed to separate for 30 minutes and the aqueous layer was drained off. The ethyl acetate layers are combined. The combined ethyl acetate mixture is extracted successively with 400 mL and 100 mL of water. If the layers do not readily separate within 30 minutes, 50 mL of brine may be added to the mixture to aid in separation of the layers. The aqueous layer is drained off. The ethyl acetate layer is then extracted with 100 mL of brine. The ethyl acetate layer is then dried over anhydrous magnesium sulfate and the solids are filtered off through a plug of activated charcoal/Supercel Hyflow. The filtrate is concentrated under reduced pressure and dried under vacuum for 18 hours to obtain 91.98 grams of an orange-brown, viscous oil (compound 67).

### EXAMPLE 126

### Conversion of Diethyl Acetal Compound 67 to 1-(2,2-Dibutyl-3-oxopropylsulfonyl)-2-((4-methoxyphenyl)methyl)benzene (29)

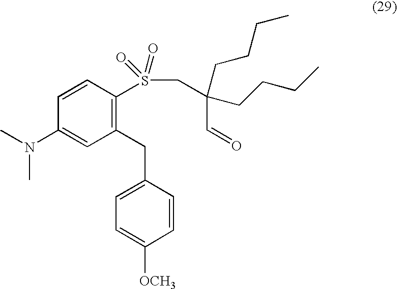

Compound 67 (36 grams dissolved in 122 mL of ethyl acetate), 300 mL acetic acid, 27.3 g of 37 wt % formaldehyde, and 50 mL of water are charged into a 500 mL 1-neck round bottom flask in a Parr Shaker. To the mixture is added 7.4 grams of 5% Pd/C (dry basis, Johnson Mathey). The reactor is purged three times with nitrogen gas and then purged three times with hydrogen gas. The reactor is pressurized to 60 psi and heated to 60° C. The temperature and pressure are held for 16 hours after which time the reactor is allowed to cool to room temperature. The reaction mixture is filtered through a pad of solka flock on a course fritted glass filter. The cake is washed twice with 40 mL of acetic acid and concentrated to dryness under reduced pressure. The solid is mixed with 100 mL ethanol and heated to 80° C. until all the solid is dissolved. To this is added 20 mL of tap water to form a homogeneous solution. The mixture is cooled to room temperature and to it is added 3 mL of ethyl acetate. A white slurry forms. The slurry is heated to 60° C. until a homogeneous solution forms. The mixture is cooled to room temperature and held for two hours. During this time compound 29 crystallizes. The solids are filtered through a coarse fritted glass filter. The cake is washed twice with 40 mL of a 20% (V/V) ethanol in water solution. The cake is dried at 40-50° C. in a vacuum oven until no weight loss is observed.

### EXAMPLE 127

### Preparation of 2-(Acetylthiomethyl)-2-butyl-4-hexenal ethylene glycol acetal, 74

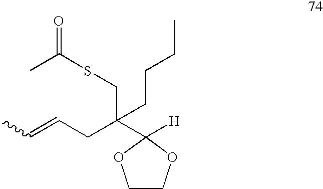

Step 1. Preparation of 2-(Acetylthiomethyl)hexanal, 72.

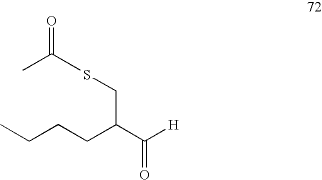

A 1 L 3-neck round bottom flask is fitted with a magnetic stir bar, a nitrogen inlet, a thermometer probe connected to a temperature monitor, a 50 mL addition funnel, and an ice-water bath. Into the flask is charged 37.0 mL of thiolacetic acid and the flask contents are cooled to 0-5° C. in the ice-water bath. To the flask is then charged 69.0 nL of butylacrolein via the addition funnel over 2 minutes. The temperature increases to a maximum of about 21° C. The reaction is cooled then to about 10° C. and the flask is charged with 0.72 mL of triethylamine. The temperature increases to about 57° C. within about one minute. Stirring continues until the temperature drops to about 15° C. The resulting product mixture contains compound 72.

Step 2. Preparation of 2-(Acetylthiomethyl)-2-butyl4-hexenal, 73.

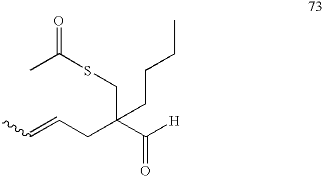

The apparatus of Step 1 of this example is further fitted with a Dean-Stark trap and a cold water condenser. The reaction flask, containing the product mixture of Step 1, is further charged with 50.0 mL of 3-buten-2-ol, 1.987 g of p-toluenesulfonic acid monohydrate, and 600 mL of toluene. The mixture is heated to about 105-110° C. with stirring for about 24 hours. During this time water, as well as some 3-buten-2-ol and toluene collect in the Dean-Stark trap. The reaction is complete when no more water distills over. If desired, an additional 0.5 equivalents of 3-buten-2-01 can be added to the flask to make up for loss from distillation. The mixture is allowed to cool to ambient temperature. The resulting aldehyde mixture contains compound 73.

Step 3. Preparation of 2-(Acetylthiomethyl)-2-butyl-4-hexenal Ethylene Glycol Acetal, 74.

The apparatus and resulting aldehyde mixture of Step 2 of this example are further charged with 31.0 mL of ethylene glycol. The mixture is heated with stirring to 105-110° C. for 2 hours. Water and toluene collect in the Dean-Stark trap during this time. The reaction is complete when no more water distills over. The mixture is cooled to ambient temperature and the reaction mixture is washed successively with 100 mL of saturated sodium bicarbonate aqueous solution, 100 mL of water, and 100 mL of brine. The solvent is removed by evaporation in a rotary evaporator. The yield is 149 grams of compound 74.

### EXAMPLE 128

### Preparation of Compound 67

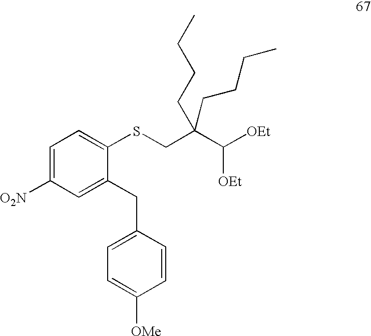

Step 1. Preparation of 2-(Acetylthiomethyl)-2-butyl-4-hexenal Diethyl Acetal 75.

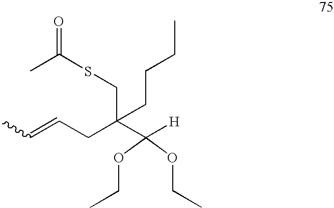

A 250 mL 3-neck round bottom flask is fitted with an overhead stirrer, a Teflon coated temperature probe, and a separatory funnel. To the flask is charged 78 g of compound 74 and 200 rnL of ethanol. The reactor is flushed with nitrogen gas and to it is charged 60 mL of triethylorthoformate. Then to the flask is added 4 grams of p-toluenesulfonic acid. The mixture is stirred at room temperature for 16 hours. The mixture is then concentrated under reduced pressure and to the flask is added 100 mL of ethyl acetate. Next is added 1.7 grams of sodium bicarbonate in 50 mL of water. The mixture is stirred for 3 minutes. The layers are allowed to separate and the aqueous layer is drained. The organic layer is filtered through a pad of sodium sulfate and the organic layer is concentrated under reduced pressure (water aspirator pressure) to afford compound 75.

Step 2. Preparation of 2-butyl-2-(thiomethyl)hexanal Diethyl Acetal, 76.

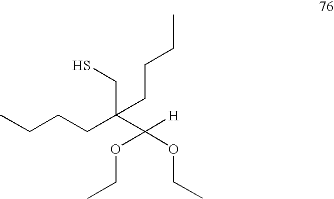

A 500 mL 3-neck round bottom flask is fitted with a condenser, a magnetic stir bar, a nitrogen inlet, a thermocouple connected to a temperature controller, and a heating mantle. The flask is purged with nitrogen gas and charged with 19.2 grams of compound 75, 96 mL of N-methyl pyrrolidone (NMP), 28.3 grams (2.5 equiv.) of p-toluenesulfonyl hydrazide, and 18 mL (3.0 equiv.) of piperidine. While stirring, the mixture is warmed to about 100° C. for 2 hours. The temperature is kept below 107° C. by removing the heat, if necessary. The mixture is cooled to ambient temperature. The product mixture contains compound 76. If desired, this reaction can be run using 2.5 equiv. of p-toluenesulfonyl hydrazide and 2.5 equiv. of piperidine.

Step 3. Preparation of Compound 67.

The equipment and product mixture of Step 2 of this example are used in this step. To the flask containing the product mixture of Step 2 is charged 13.46 grams of compound 33 and 11.2 mL of 50% (w/w) aqueous NaOH. The mixture is heated to 100° C. with mixing and held at that temperature for 2.5 hours. The mixture is cooled to ambient temperature and to the flask is added 100 mL of ethyl acetate. This mixture is washed with 100 mL of water. The aqueous layer is separated and washed with 100 mL of ethyl acetate. The ethyl acetate layers are combined and washed in succession with 3×100 mL of water and with 2×50 mL of brine. The organic layer is dried over magnesium sulfate and the solvent is removed under vacuum in a rotary evaporator. The yield is 26 grams of compound 67 as a reddish brown oil.

### EXAMPLE 129

### Differential Scanning Calorimetry (DSC)

DSC experiments are performed either on a Perkin Elmer Pyris 7 Differential Scanning Calorimeter or on a TA Instruments Differential Scanning Calorimeter with 5-10 mg samples hermetically sealed in a standard aluminum pan (40 microliters) with a single hole punched in the lid. An empty pan of the same type is used as a reference. The heating rate is 10° C./minute with dry nitrogen purge. FIG. 4 shows typical DSC thermograms for Form I (plot(a)) and Form II (plot(b)) of compound 41.

### EXAMPLE 130

### X-Ray Powder Diffraction Patterns

X-ray powder diffraction experiments are conducted on an Inel theta/theta diffraction system equipped with a 2 kW normal focus X-ray tube (copper). X-ray scatter data are collected from 0 to 80° 2 theta. Samples are run in bulk configuration. Data are collected and analyzed on a Dell computer running Inel's software. In at least one case, samples are placed in a glass capillary tube and ends are sealed to prevent loss of solvent. The capillary is mounted on a special adapter in the path of the X-ray beam and data were collected.

Alternatively, the X-ray diffraction experiments are conducted on a system comprising a Siemens D5000 diffraction system equipped with a 2 kW normal focus X-ray tube (copper). The system is equipped with an autosampler system with a theta-theta sample orientation. Data collection and analysis is performed on a MS-Windows computer with Siemens' proprietary software.

FIG. 1 shows typical X-ray powder diffraction patterns for Form I (plot (a)) and Form II (plot(b)) of compound 41. Table x-130 shows a summary comparison of prominent X-ray powder diffraction peaks for Form I and Form II.

TABLE X-130

| | |
|---|---|
| Form I | Form II |

| | Relative | | Relative |
|---|---|---|---|
| | Peak | | Peak |
| 2-Theta | Intensity | 2-Theta | Intensity |
| Value | (%) | Value | (%) |
| 7.203 | 15.0665 | 9.1962 | 18.6166 |
| 8.45 | 29.0688 | 12.277 | 29.2318 |
| 9.726 | 37.1457 | 12.584 | 8.39048 |
| 11.205 | 49.0207 | 12.833 | 7.67902 |
| 11.786 | 10.8439 | 13.872 | 100 |
| 12.51 | 15.9267 | 14.286 | 77.5682 |
| 13.342 | 11.0306 | 15.168 | 7.54978 |
| 14.25 | 16.3005 | 15.641 | 16.0194 |
| 14.859 | 16.1351 | 15.935 | 11.4935 |
| 15.526 | 43.0987 | 16.138 | 16.6656 |
| 15.874 | 25.424 | 16.399 | 36.1255 |
| 16.309 | 14.278 | 16.544 | 77.6935 |
| 17.121 | 14.1898 | 17.094 | 13.1102 |
| 17.498 | 13.173 | 17.645 | 38.4531 |
| 18.542 | 99.3626 | 18.511 | 33.0226 |
| 19.354 | 85.1982 | 18.826 | 91.0787 |
| 19.789 | 16.7251 | 19.128 | 25.2644 |
| 20.34 | 39.3083 | 19.327 | 18.8639 |
| 20.891 | 27.5965 | 19.906 | 38.7122 |
| 21.297 | 16.2266 | 20.085 | 12.7865 |
| 22.022 | 26.6845 | 20.23 | 10.2004 |
| 23.304 | 42.0171 | 21.00 | 8.58433 |
| 25.125 | 17.2159 | 21.48 | 47.6981 |
| 25.734 | 18.2944 | 21.729 | 33.6048 |
| 27.503 | 25.8376 | 22.089 | 12.1403 |
| 32.056 | 12.7407 | 22.4 | 10.0712 |
| 35.188 | 22.4211 | 22.748 | 13.3041 |
| 40.166 | 16.7913 | 22.959 | 14.5971 |
| | | 23.22 | 13.498 |
| | | 23.472 | 17.8224 |
| | | 23.965 | 16.9247 |
| | | 24.553 | 16.8594 |
| | | 25.038 | 9.6835 |
| | | 25.299 | 13.0904 |
| | | 25.626 | 13.9503 |
| | | 25.767 | 14.9202 |
| | | 25.887 | 11.2996 |
| | | 26.343 | 18.1531 |
| | | 26.873 | 9.87736 |
| | | 27.941 | 15.1787 |
| | | 28.228 | 15.4437 |
| | | 28.815 | 11.2996 |
| | | 29.475 | 13.7532 |
| | | 34.758 | 21.773 |
| | | 40.176 | 21.0731 |

### EXAMPLE 131

### Fourier Transform Infrared Spectra

The Fourier transform infrared (FTIR) spectra for Form I and Form II of compound 41 are obtained using a Bio-Rad FTS45 Fourier-transform infrared spectrometer equipped with a micro-ATR (attenuated total reflectance) beam condensing accessory (IBM Corporation) mounted in the sample compartment of the instrument. The sample compartment and optical bench of the spectrometer is under a nitrogen purge. The software used for operating the instrument and collecting the spectrum is Bio-Rad's Windows 98-based Win-IR software. The spectra are obtained using an 8-wavenumber resolution and 16 scans.

A small amount of sample is placed onto one side of a 5×10×1 mm KRS5 (a type of infrared transmitting material commonly used in the IR world) ATR crystal, and lightly tamped with a stainless steel micro spatula in order to ensure good contact of the sample with the face of the crystal. The crystal is mounted into the ATR beam-condensing accessory, and the sample compartment allowed to purge for a few minutes to remove water vapor and carbon dioxide (their presence reduces the quality of the spectrum). This can be monitored on the screen of the operating console, and when down to an acceptable level, the 16 scans are collected to produce an interferogram. Prior to analyzing the sample, a clean KRS5 crystal is mounted in the ATR accessory and a background interferogram collected. The purge time and number of scans for collecting the background shouid be the same as will be used for analyzing the sample.

The Fourier-transform of the resulting interferogram is automatically done and the spectrum appears on the screen. The resulting spectrum is then smoothed and baseline corrected, if necessary, then ATR corrected to obtain a spectrum that is comparable to an absorption or transmission spectrum.

FIG. 2 shows typical FTIR spectra for Form I (plot (a)) and Form II (plot (b)) of compound 41. Table X-131 shows a summary comparison of prominent FTIR peaks for Form I and Form II.

TABLE X-131

| Form I Peaks | Form II Peaks |
|---|---|
| (cm⁻¹) | (cm⁻¹) |
| 3163 | 3250 |
| 2870 | 2885 |
| 1596 | 1600 |
| 1300 | 1288 |
| 1239 | 1225 |
| 1182 | 1172 |
| 1055 | 1050 |
| 986 | 990 |
| 855 | 858 |
| 825 | 837 |
| 627 | 620 |

### EXAMPLE 132

### Solid-State Carbon-13 NMR Analysis

Solid-state NMR.

Cross-polarization magic-angle spinning (CPMAS) ¹³C NMR spectra were collected on a Monsanto-built spectrometer operating at a proton resonance frequency of 127.0 MHz. Samples were spun at the magic angle with respect to the magnetic field in a double-bearing rotor system at a rate of 3 kHz. CPMAS ¹³C NMR spectra were obtained at 31.9 MHz following 2-ms matched, 50-kHz 1H-¹³C cross-polarization contacts. High-power proton dipolar decoupling (H₁(H)=65-75 kHz) was used during data acquisition. Residual spinning sidebands were suppressed using the Total Suppression of Sidebands (TOSS) method. In each experiment, approximately 219 mg of Form I and approximately 142 mg Form II are used.

FIG. 3 shows typical solid-state ¹³C nuclear magnetic resonance (NMR) spectra for Form I (plot (a)) and Form II (plot (b)) of compound 41. Table X-132 shows a summary comparison of prominent solid-state ¹³C NMR peaks for Form I and Form II.

TABLE X-132

| Form I (ppm) | Form II (ppm) |
|---|---|
| 158.55 | 157.971 |
| 151.712 | 142.325 |
| 145.986 | 137.172 |
| 140.852 | 134.043 |
| 136.628 | 127.232 |
| 133.489 | 125.390 |
| 128.151 | 118.212 |
| 120.052 | 113.057 |
| 115.266 | 106.615 |
| 113.241 | 76.795 |
| 109.928 | 68.512 |
| 76.795 | 57.100 |
| 68.860 | 47.712 |
| 54.523 | 43.661 |
| 46.239 | 37.951 |
| 43.847 | 21.942 |
| 40.901 | 14.763 |
| 24.519 | 13.281 |
| 14.395 | |
| 3.351 | |

### EXAMPLE 133

### Water Uptake Experiments

Water sorption experiments are performed on a Dynamic Vapor Sorption (DVS) apparatus (DVS-1000 manufactured by Surface Measurements Systems, Inc.). Experiments are performed at 25° C. by initially drying the material of interest (about 10 mg sample) from 30% relative humidity (RH) (ambient room condition) to about 9% RH in a stepwise fashion (10% RH step) by purging with dry nitrogen until no further weight change was observed. The samples are then exposed to a stepwise (10% RH steps) increase in RH from about 0 to about 90% RH. Each successive step is initiated when the change in weight over time at the relative humidity was less than 0.0003% ((dm/dt)/m₀×100, where m is mass in mg, mo is initial mass, and t is time in minutes). The sample is then taken through the reverse of the stepwise % RH increase. The data are collected on a computer and analyzed using SMS' proprietary MS-Excel macro interface software. 10 FIG. 5 shows typical water sorption isotherm results for Form I (plot (a)) and Form II (plot (b)) of compound 41. Table X-133 shows a summary comparison of water sorption and desorption isotherms for Form I and Form II at 25° C.

TABLE X-133

| | | |
|---|---|---|
| | | Desorption |
| | Sorption % | % Weight |
| % RH at 25° C. | Weight Change | Change |

Form I

| | | |
|---|---|---|
| 0.45 | 0.057 | 0.057 |
| 9.2 | 0.9575 | 0.997 |
| 20.05 | 2.016 | 2.1025 |
| 29.75 | 3.4105 | 3.599 |
| 39.4 | 4.282 | 4.743 |
| 49.55 | 4.928 | 5.321 |
| 59.4 | 5.356 | 5.726 |
| 69.05 | 5.706 | 6.054 |
| 78.8 | 6.109 | 6.357 |
| 88.5 | 6.734 | 6.734 |

Form II

| | | |
|---|---|---|
| 1.3 | −0.02695 | −0.02695 |
| 9.35 | 0.04715 | 0.04235 |
| 20.25 | 0.10585 | 0.09715 |
| 29.75 | 0.13755 | 0.14435 |
| 39.55 | 0.1809 | 0.1866 |
| 49.7 | 0.2386 | 0.2636 |
| 59.5 | 0.304 | 0.331 |
| 59.1 | 0.3945 | 0.3983 |
| 78.65 | 0.4695 | 0.4849 |
| 88.5 | 0.6446 | 0.6446 |

### EXAMPLE 134

Tale X-134 illustrates specific examples of the combinations of the present invention wherein the combination comprises a first amount of an ASBT inhibitor and a second amount of an HMG Co-A reductase inhibitor, and wherein the first and second amounts together comprise an anti-hyperlipidemic condition effective amount or an anti-atherosclerotic condition effective amount of the compounds.

TABLE X-134

| Combination | ASBT | |
|---|---|---|
| Number | Inhibitor | Statin |
| 1 | A-1 | B-1 |
| 2 | A-1 | B-2 |
| 3 | A-1 | B-3 |
| 4 | A-1 | B-4 |
| 5 | A-1 | B-5 |
| 6 | A-1 | B-6 |
| 7 | A-1 | B-7 |
| 8 | A-1 | B-8 |
| 9 | A-1 | B-9 |
| 10 | A-2 | B-1 |
| 11 | A-2 | B-2 |
| 12 | A-2 | B-3 |
| 13 | A-2 | B-4 |
| 14 | A-2 | B-5 |
| 15 | A-2 | B-6 |
| 16 | A-2 | B-7 |
| 17 | A-2 | B-8 |
| 18 | A-2 | B-9 |
| 19 | A-3 | B-1 |
| 20 | A-3 | B-2 |
| 21 | A-3 | B-3 |
| 22 | A-3 | B-4 |
| 23 | A-3 | B-5 |
| 24 | A-3 | B-6 |
| 25 | A-3 | B-7 |
| 26 | A-3 | B-8 |
| 27 | A-3 | B-9 |
| 28 | A-4 | B-1 |
| 29 | A-4 | B-2 |
| 30 | A-4 | B-3 |
| 31 | A-4 | B-4 |
| 32 | A-4 | B-5 |
| 33 | A-4 | B-6 |
| 34 | A-4 | B-7 |
| 35 | A-4 | B-8 |
| 36 | A-4 | B-9 |
| 37 | A-5 | B-1 |
| 38 | A-5 | B-2 |
| 39 | A-5 | B-3 |
| 40 | A-5 | B-4 |
| 41 | A-5 | B-5 |
| 42 | A-5 | B-6 |
| 43 | A-5 | B-7 |
| 44 | A-5 | B-8 |
| 45 | A-5 | B-9 |
| 46 | A-7 | B-1 |
| 47 | A-7 | B-2 |
| 48 | A-7 | B-3 |
| 49 | A-7 | B-4 |
| 50 | A-7 | B-5 |
| 51 | A-7 | B-6 |
| 52 | A-7 | B-7 |
| 53 | A-7 | B-8 |
| 54 | A-7 | B-9 |
| 55 | A-8 | B-1 |
| 56 | A-8 | B-2 |
| 57 | A-8 | B-3 |
| 58 | A-8 | B-4 |
| 59 | A-8 | B-5 |
| 60 | A-8 | B-6 |
| 61 | A-8 | B-7 |
| 62 | A-8 | B-8 |
| 63 | A-8 | B-9 |
| 64 | A-9 | B-1 |
| 65 | A-9 | B-2 |
| 66 | A-9 | B-3 |
| 67 | A-9 | B-4 |
| 68 | A-9 | B-5 |
| 69 | A-9 | B-6 |
| 70 | A-9 | B-7 |
| 71 | A-9 | B-8 |
| 72 | A-9 | B-9 |
| 73 | A-10 | B-1 |
| 74 | A-10 | B-2 |
| 75 | A-10 | B-3 |
| 76 | A-10 | B-4 |
| 77 | A-10 | B-5 |
| 78 | A-10 | B-6 |
| 79 | A-10 | B-7 |
| 80 | A-10 | B-8 |
| 81 | A-10 | B-9 |
| 82 | A-11 | B-1 |
| 83 | A-11 | B-2 |
| 84 | A-11 | B-3 |
| 85 | A-11 | B-4 |
| 86 | A-11 | B-5 |
| 87 | A-11 | B-6 |
| 88 | A-11 | B-7 |
| 89 | A-11 | B-8 |
| 90 | A-11 | B-9 |
| 91 | A-12 | B-1 |
| 92 | A-12 | B-2 |
| 93 | A-12 | B-3 |
| 94 | A-12 | B-4 |
| 95 | A-12 | B-5 |
| 96 | A-12 | B-6 |
| 97 | A-12 | B-7 |
| 98 | A-12 | B-8 |
| 99 | A-12 | B-9 |
| 100 | A-13 | B-1 |
| 101 | A-13 | B-2 |
| 102 | A-13 | B-3 |
| 103 | A-13 | B-4 |
| 104 | A-13 | B-5 |
| 105 | A-13 | B-6 |
| 106 | A-13 | B-7 |
| 107 | A-13 | B-8 |
| 108 | A-13 | B-9 |
| 109 | A-14 | B-1 |
| 110 | A-14 | B-2 |
| 111 | A-14 | B-3 |
| 112 | A-14 | B-4 |
| 113 | A-14 | B-5 |
| 114 | A-14 | B-6 |
| 115 | A-14 | B-7 |
| 116 | A-14 | B-8 |
| 117 | A-14 | B-9 |
| 118 | A-15 | B-1 |
| 119 | A-15 | B-2 |
| 120 | A-15 | B-3 |
| 121 | A-15 | B-4 |
| 122 | A-15 | B-5 |
| 123 | A-15 | B-6 |
| 124 | A-15 | B-7 |
| 125 | A-15 | B-8 |
| 126 | A-15 | B-9 |

The examples herein can be performed by substituting the generically or specifically described reactants and/or operating conditions of this invention for those used in the preceding examples.

In view of the above, it will be seen that the several objects of the invention are achieved. As various changes could be made in the above methods, combinations and compositions of the present invention without departing from the scope of the invention, it is intended that all matter contained in the above description be interpreted as illustrative and not in a limiting sense. All documents mentioned in this application are expressly incorporated by reference as if fully set forth at length.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

What we claim is:

1. A method for the prophylaxis or treatment of a hyperlipidemic condition or disorder in a subject which comprises administering a first amount of an apical sodium co-dependent bile acid transporter inhibitor and a second amount of an HMG Co-A reductase inhibitor wherein:
the apical sodium co-dependent bile acid transporter inhibitor is selected from the group consisting of: 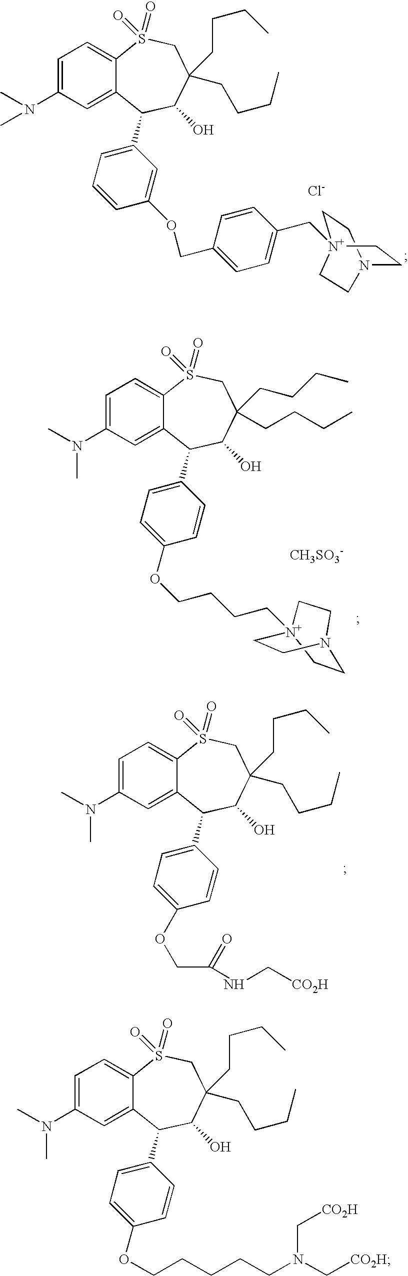 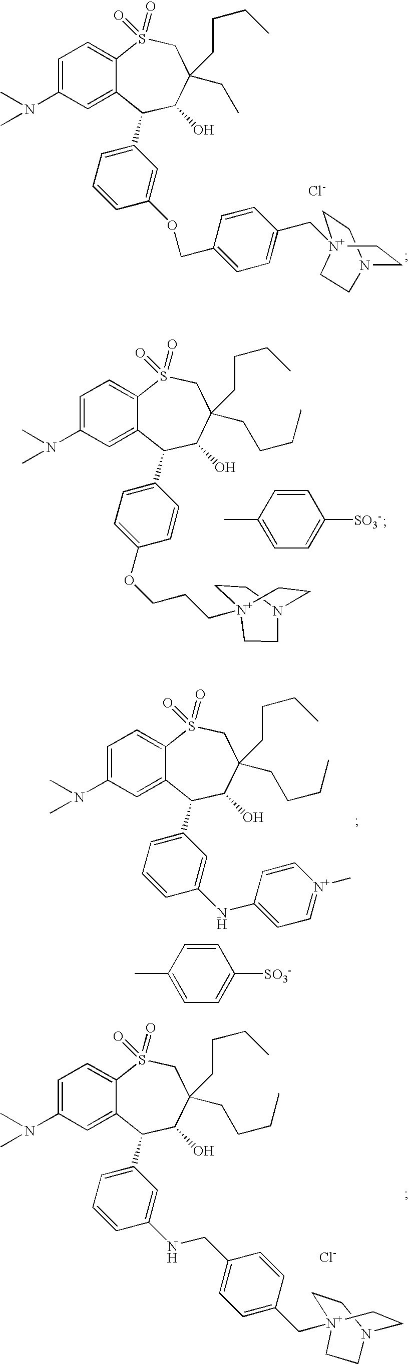 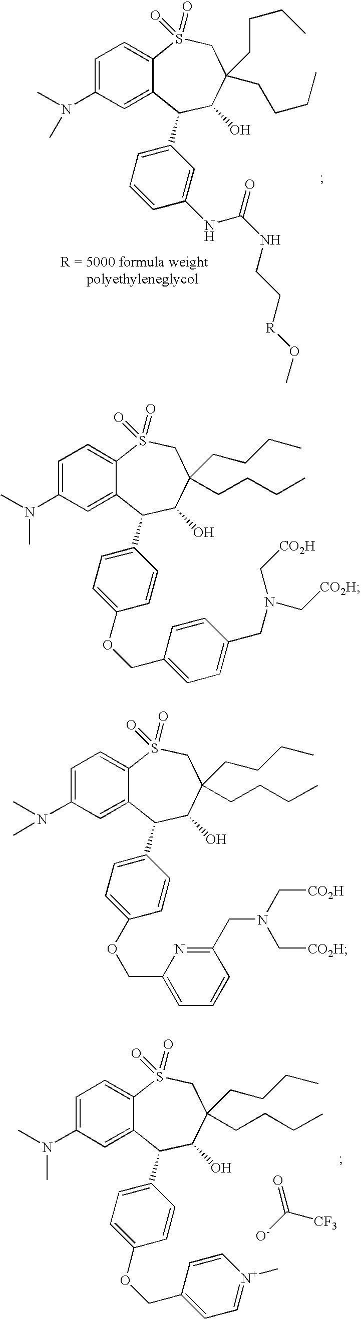 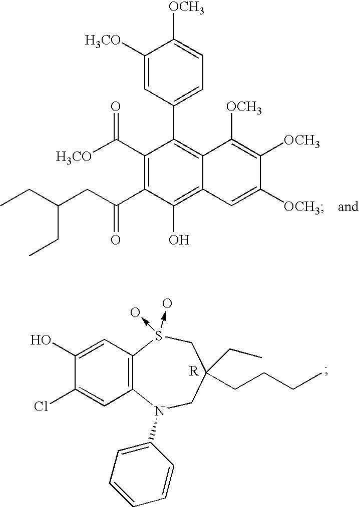
and the pharmaceutically acceptable salts, esters, and prodrugs thereof; and
the first and second amounts of said inhibitors together comprise a therapeutically effective amount of said inhibitors.

2. The method of claim 1 wherein the apical sodium co-dependent bile acid transporter inhibitor comprises 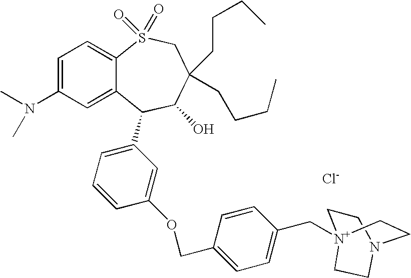
a pharmaceutically acceptable salt, ester or prodrug thereof.

3. The method of claim 1 wherein the apical sodium co-dependent bile acid transporter inhibitor comprises 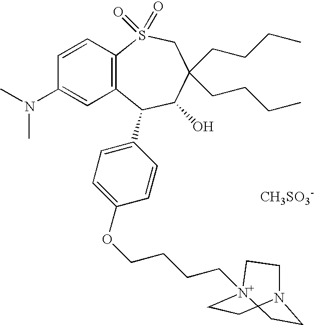
or a pharmaceutically acceptable salt, ester or prodrug thereof.

4. The method of claim 1 wherein the apical sodium co-dependent bile acid transporter inhibitor comprises 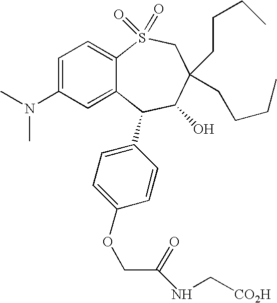
or a pharmaceutically acceptable salt, ester or prodrug thereof.

5. The method of claim 1 wherein the apical sodium co-dependent bile acid transporter inhibitor comprises 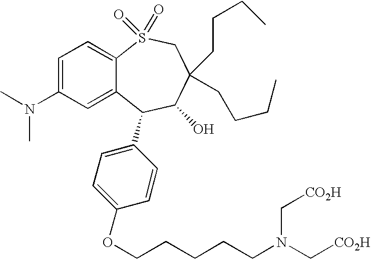
or a pharmaceutically acceptable salt, ester or prodrug thereof.

6. The method of claim 1 wherein the apical sodium co-dependent bile acid transporter inhibitor comprises 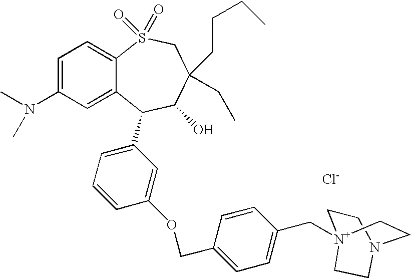
or a pharmaceutically acceptable salt, ester or prodrug thereof.

7. The method of claim 1 wherein the apical sodium co-dependent bile acid transporter inhibitor comprises 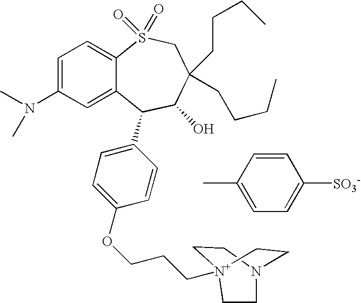
or a pharmaceutically acceptable salt, ester or prodrug thereof.

8. The method of claim 1 wherein the apical sodium co-dependent bile acid transporter inhibitor comprises 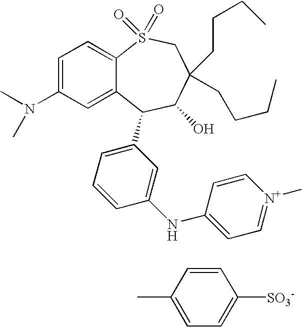
or a pharmaceutically acceptable salt, ester or prodrug thereof.

9. The method of claim 1 wherein the apical sodium co-dependent bile acid transporter inhibitor comprises 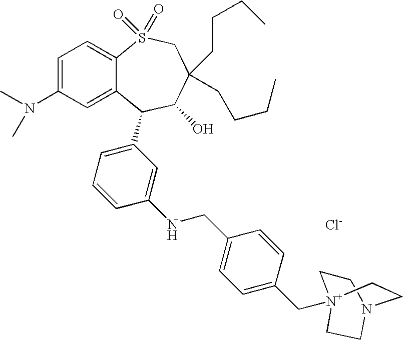
or a pharmaceutically acceptable salt, ester or prodrug thereof.

10. The method of claim 1 wherein the apical sodium co-dependent bile acid transporter inhibitor comprises 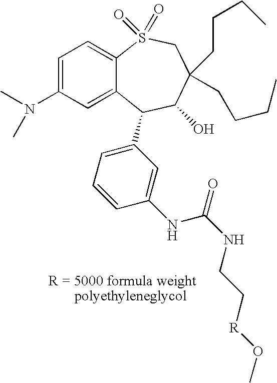
or a pharmaceutically acceptable salt, ester or prodrug thereof.

11. The method of claim 1 wherein the apical sodium co-dependent bile acid transporter inhibitor comprises 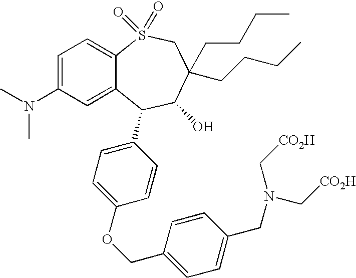
or a pharmaceutically acceptable salt, ester or prodrug thereof.

12. The method of claim 1 wherein the apical sodium co-dependent bile acid transporter inhibitor comprises 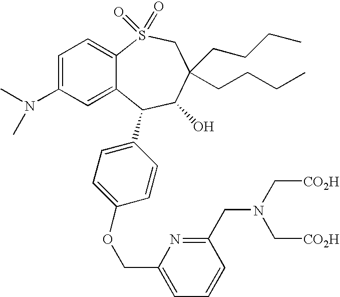
or a pharmaceutically acceptable salt, ester or prodrug thereof.

13. The method of claim 1 wherein the apical sodium co-dependent bile acid transporter inhibitor comprises 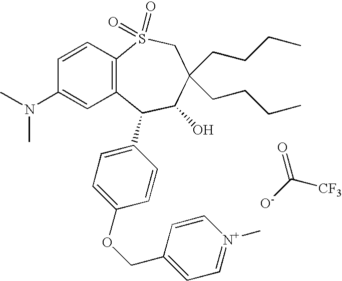
or a pharmaceutically acceptable salt, ester or prodrug thereof.

14. The method of claim 1 wherein the apical sodium co-dependent bile acid transporter inhibitor comprises 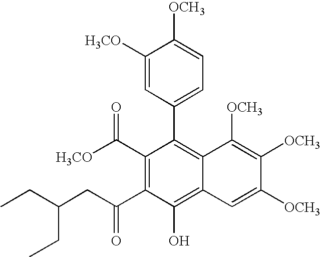
or a pharmaceutically acceptable salt, ester or prodrug thereof.

15. The method of claim 1 wherein the apical sodium co-dependent bile acid transporter inhibitor comprises 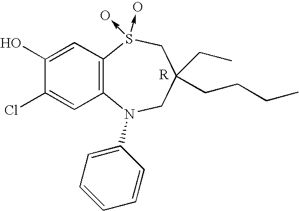
or a pharmaceutically acceptable salt, ester or prodrug thereof.

16. The method of claim 1 wherein the HMG Co-A reductase inhibitor is selected from the group consisting of mevastatin, lovastatin, simvastatin, pravastatin, fluvastatin, cerivastatin, atorvastatin, ZD-4522, and the pharmaceutically acceptable salts, esters, conjugate acids, and prodrugs thereof.

17. The method of claim 1 wherein the HMG Co-A reductase inhibitor is selected from the group consisting of atorvastatin, simvastatin, pravastatin, ZD-4522, and the pharmaceutically acceptable salts, esters, conjugate acids, and prodrugs thereof.

18. The method of claim 1 wherein the HMG Co-A reductase inhibitor comprises mevastatin, or a pharmaceutically acceptable salt, ester or prodrug thereof.

19. The method of claim 1 wherein the HMG Co-A reductase inhibitor comprises atorvastatin, or a pharmaceutically acceptable salt, ester or prodrug thereof.

20. The method of claim 1 wherein the HMG Co-A reductase inhibitor comprises simvastatin, or a pharmaceutically acceptable salt, ester or prodrug thereof.

21. The method of claim 1 wherein the HMG Co-A reductase inhibitor comprises pravastatin, or a pharmaceutically acceptable salt, ester or prodrug thereof.

22. The method of claim 1 wherein the HMG Co-A reductase inhibitor comprises lovastatin, or a pharmaceutically acceptable salt, ester or prodrug thereof.

23. The method of claim 1 wherein the HMG Co-A reductase inhibitor comprises cerivastatin, or a pharmaceutically acceptable salt, ester or prodrug thereof.

24. The method of claim 1 wherein the HMG Co-A reductase inhibitor comprises fluvastatin, or a pharmaceutically acceptable salt, ester or prodrug thereof.

25. The method of claim 1 wherein the HMG Co-A reductase inhibitor comprises ZD-4522, or a pharmaceutically acceptable salt, ester, conjugate acid, or prodrug thereof.

26. The method of claim 1 wherein the HMG Co-A reductase inhibitor comprises NK-104, or a pharmaceutically acceptable salt, ester, conjugate acid, or prodrug thereof.

27. The method of claim 1 wherein the apical sodium co-dependent bile acid transporter inhibitor comprises 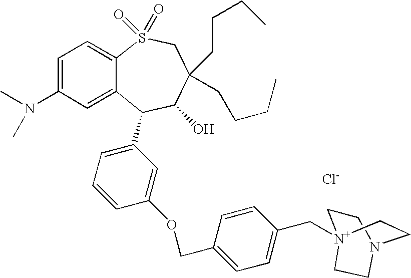
or a pharmaceutically acceptable salt, ester or prodrug thereof; and
the HMG Co-A reductase inhibitor is selected from the group consisting of mevastatin, lovastatin, simvastatin, pravastatin, fluvastatin, cerivastatin, atorvastatin, ZD-4522, NK-104, and the pharmaceutically acceptable salts, esters, conjugate acids, and prodrugs thereof.

28. The method of claim 27 wherein the apical sodium co-dependent bile acid transporter inhibitor comprises the 4R,5R enantiomer of 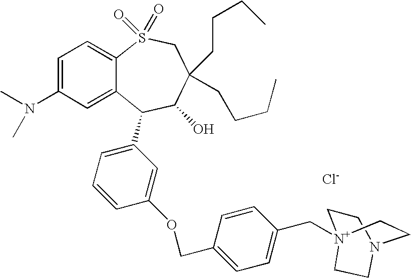
or a pharmaceutically acceptable salt, ester or prodrug thereof.

29. The method of claim 27 wherein the apical sodium co-dependent bile acid transporter inhibitor comprises the racemate of 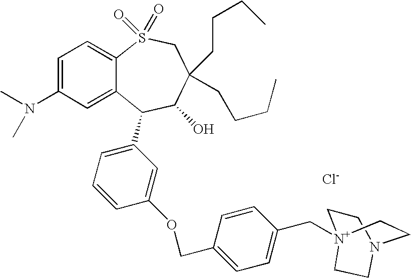
or a pharmaceutically acceptable salt, ester or prodrug thereof.

30. The method of claim 28 wherein the HMG Co-A reductase inhibitor is selected from the group consisting of atorvastatin, simvastatin, pravastatin, ZD-4522, and the pharmaceutically acceptable salts, esters, conjugate acids, and prodrugs thereof.

31. The method of claim 28 wherein the HMG Co-A reductase inhibitor comprises mevastatin, or a pharmaceutically acceptable salt, ester or prodrug thereof.

32. The method of claim 28 wherein the HMG Co-A reductase inhibitor comprises lovastatin, or a pharmaceutically acceptable salt, ester or prodrug thereof.

33. The method of claim 28 wherein the HMG Co-A reductase inhibitor comprises simvastatin, or a pharmaceutically acceptable salt, ester or prodrug thereof.

34. The method of claim 28 wherein the HMG Co-A reductase inhibitor comprises pravastatin, or a pharmaceutically acceptable salt, ester or prodrug thereof.

35. The method of claim 28 wherein the HMG Co-A reductase inhibitor comprises filuvastatin, or a pharmaceutically acceptable salt, ester or prodrug thereof.

36. The method of claim 28 wherein the HMG Co-A reductase inhibitor comprises cerivastatin, or a pharmaceutically acceptable salt, ester or prodrug thereof.

37. The method of claim 28 wherein the HMG Co-A reductase inhibitor comprises atorvastatin, or a pharmaceutically acceptable salt, ester or prodrug thereof.

38. The method of claim 28 wherein the HMG Co-A reductase inhibitor comprises ZD-4522, or a pharmaceutically acceptable salt, ester, conjugate acid, or prodrug thereof.

39. The method of claim 28 wherein the HMG Co-A reductase inhibitor comprises NK-104, or a pharmaceutically acceptable salt, ester, conjugate acid, or prodrug thereof.

40. The method of claim 28 wherein the apical sodium co-dependent bile acid transporter inhibitor and the HMG Co-A reductase inhibitor are administered in a sequential manner.

41. The method of claim 28 wherein the apical sodium co-dependent bile acid transporter inhibitor and the HMG Co-A reductase inhibitor are administered in a substantially simultaneous manner.

42. The method of claim 28 wherein the weight ratio of apical sodium co-dependent bile acid transporter inhibitor to HMG Co-A reductase inhibitor administered is between about 1:50 to about 3:1.

43. The method of claim 28 wherein said apical sodium co-dependent bile acid transporter inhibitor is administered in a daily dose ranging from about 0.008 mg to about 100 mg,and said HMG Co-A reductase inhibitor is administered in a daily dose ranging from about 0.05 mg to about 100 mg.

44. The method of claim 28 wherein said apical sodium co-dependent bile acid transporter inhibitor is administered in a daily dose range from about 0.08 mg to about 100 mg.

45. The method of claim 28 wherein the HMG Co-A reductase inhibitor is administered in a daily dose range from about 0.05 mg to about 100 mg.

46. A composition comprising a first amount of an apical sodium co-dependent bile acid transporter inhibitor selected from the group consisting of 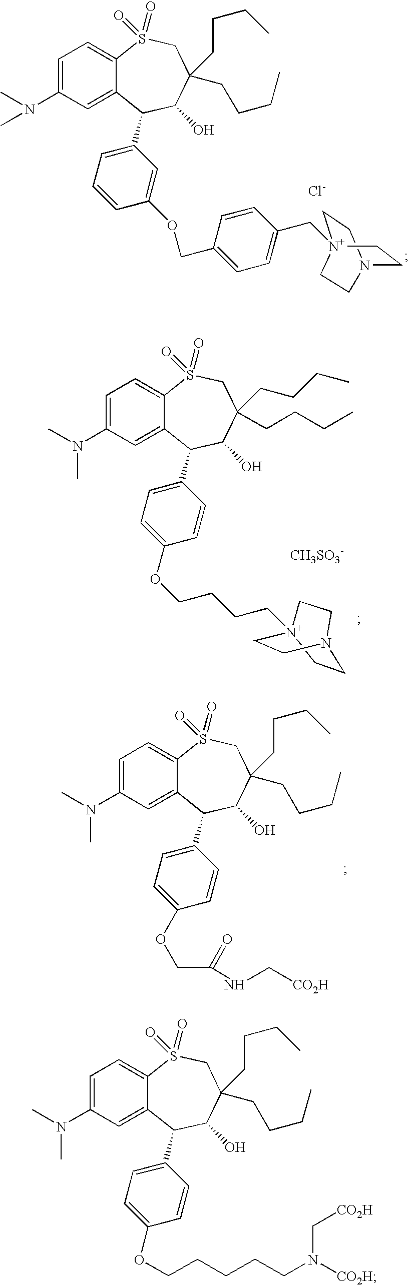 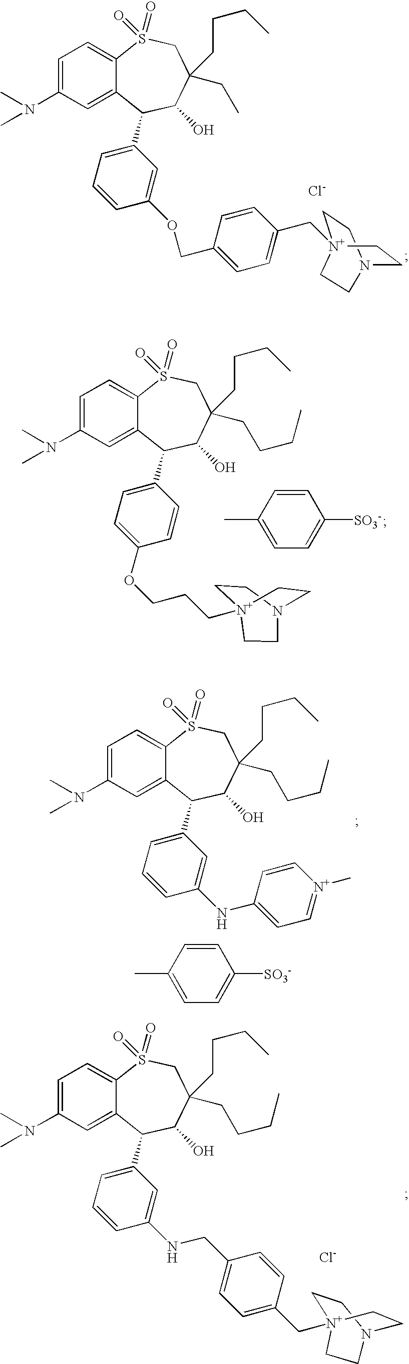 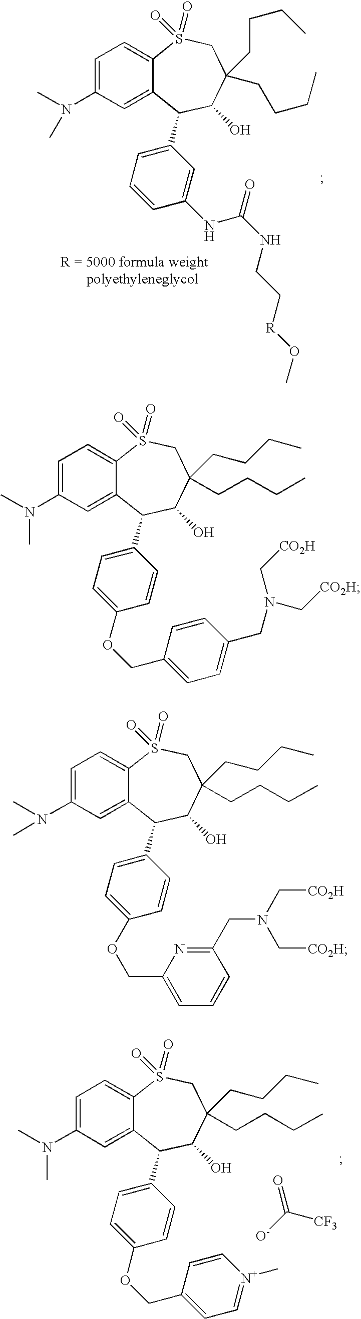 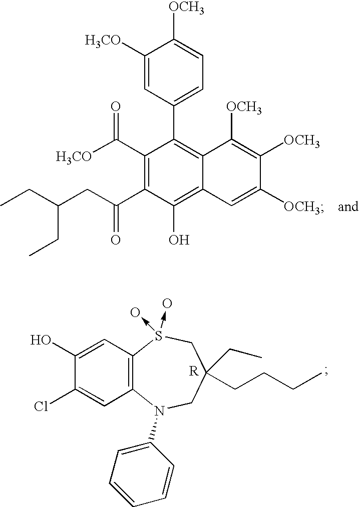
and the pharmaceutically acceptable salts, esters and prodrugs thereof;
a second amount of the HMG Co-A reductase inhibitor, or a pharmaceutically acceptable salt, ester, conjugate acid, or prodrug thereof; and
a pharrnaceutically acceptable carrier;
wherein the first and second amounts of said inhibitors together comprise a therapeutically effective amount of said inhibitors.

47. The composition of claim 46 wherein the apical sodium co-dependent bile acid transporter inhibitor comprises 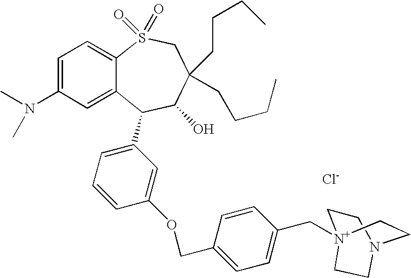
or a pharmaceutically acceptable salt, ester or prodrug thereof.

48. The composition of claim 46 wherein the apical sodium co-dependent bile acid transporter inhibitor comprises 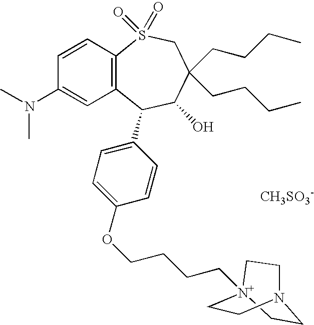
or a pharmaceutically acceptable salt, ester or prodrug thereof.

49. The composition of claim 46 wherein the apical sodium co-dependent bile acid transporter inhibitor comprises 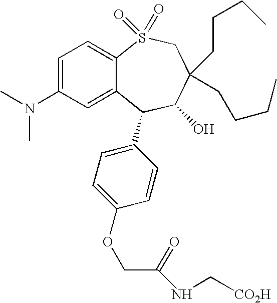
or a pharmaceutically acceptable salt, ester or prodrug thereof.

50. The composition of claim 46 wherein the apical sodium co-dependent bile acid transporter inhibitor comprises 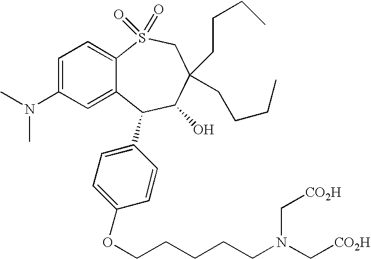
or a pharmaceutically acceptable salt, ester or prodrug thereof.

51. The composition of claim 46 wherein the apical sodium co-dependent bile acid transporter inhibitor comprises 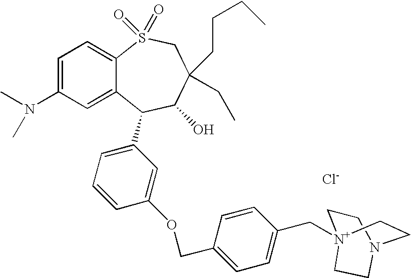
or a pharmaceutically acceptable salt, ester or prodrug thereof.

52. The composition of claim 46 wherein the apical sodium co-dependent bile acid transporter inhibitor comprises 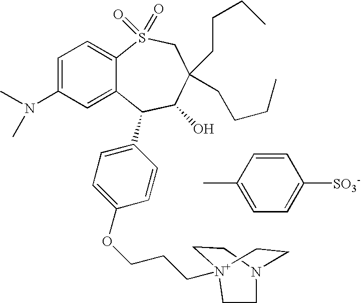
or a pharmaceutically acceptable salt, ester or prodrug thereof.

53. The composition of claim 46 wherein the apical sodium co-dependent bile acid transporter inhibitor comprises 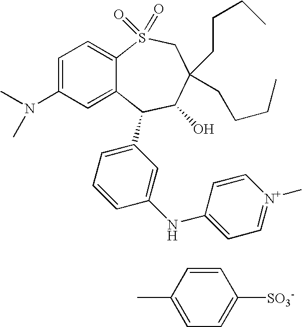
or a pharmaceutically acceptable salt, ester or prodrug thereof.

54. The composition of claim 46 wherein the apical sodium co-dependent bile acid transporter inhibitor comprises 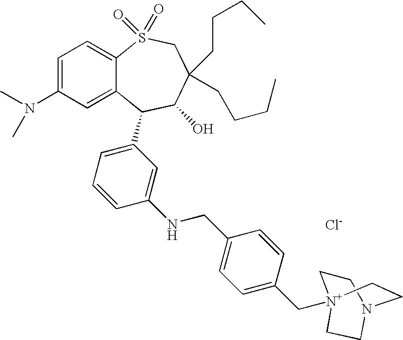
or a pharmaceutically acceptable salt, ester or prodrug thereof.

55. The composition of claim 46 wherein the apical sodium co-dependent bile acid transporter inhibitor comprises 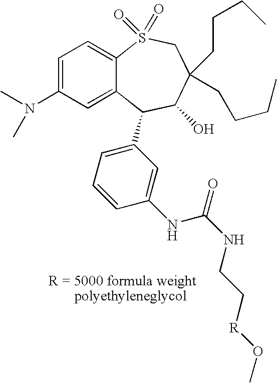
or a pharmaceutically acceptable salt, ester or prodrug thereof.

56. The composition of claim 46 wherein the apical sodium co-dependent bile acid transporter inhibitor comprises 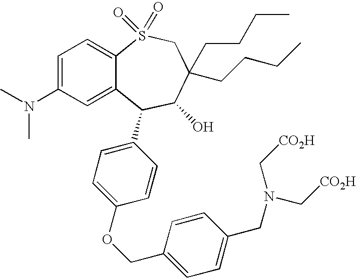
or a pharmaceutically acceptable salt, ester or prodrug thereof.

57. The composition of claim 46 wherein the apical sodium co-dependent bile acid transporter inhibitor comprises 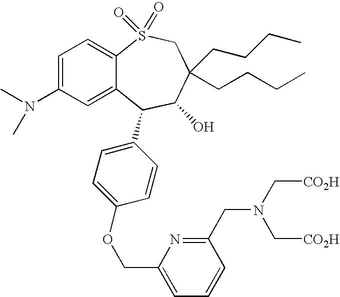
or a pharmaceutically acceptable salt, ester or prodrug thereof.

58. The composition of claim 46 wherein the apical sodium co-dependent bile acid transporter inhibitor comprises 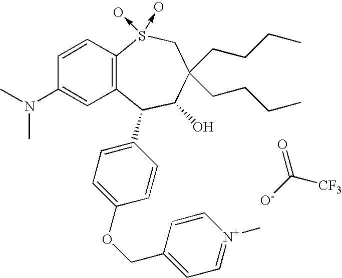
or a pharmaceutically acceptable salt, ester or prodrug thereof.

59. The composition of claim 46 wherein the apical sodium co-dependent bile acid transporter inhibitor comprises 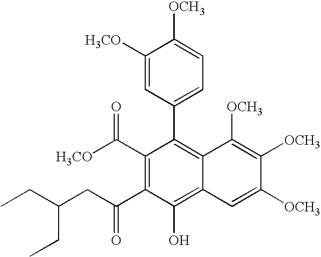
or a pharmaceutically acceptable salt, ester or prodrug thereof.

60. The composition of claim 46 wherein the apical sodium co-dependent bile acid transporter inhibitor comprises 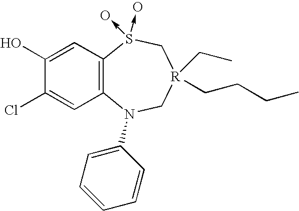
or a pharmaceutically acceptable salt, ester or prodrug thereof.

61. The composition of claim 46 wherein the HMG Co-A reductase inhibitor is selected from the group consisting of mevastatin, lovastatin, simvastatin, pravastatin, fluvastatin, cerivastatin, atorvastatin, ZD-4522, NK-104, and the pharmaceutically acceptable salts, esters, conjugate acids, and prodrugs thereof.

62. The composition of claim 46 wherein the HMG Co-A reductase inhibitor is selected from the group consisting of atorvastatin, simvastatin, pravastatin, ZD-4522, and the pharmaceutically acceptable salts, esters, conjugate acids, and prodrugs thereof.

63. The composition of claim 46 wherein the HMG Co-A reductase inhibitor comprises mevastatin, or a pharmaceutically acceptable salt, ester or prodrug thereof.

64. The composition of claim 46 wherein the HMG Co-A reductase inhibitor comprises atorvastatin, or a pharmaceutically acceptable salt, ester or prodrug thereof.

65. The composition of claim 46 wherein the HMG Co-A reductase inhibitor comprises simvastatin, or a pharmaceutically acceptable salt, ester or prodrug thereof.

66. The composition of claim 46 wherein the HMG Co-A reductase inhibitor comprises pravastatin, or a pharmaceutically acceptable salt, ester or prodrug thereof.

67. The composition of claim 46 wherein the HMG Co-A reductase inhibitor comprises lovastatin, or a pharmaceutically acceptable salt, ester or prodrug thereof.

68. The composition of claim 46 wherein the HMG Co-A reductase inhibitor comprises cerivastatin, or a pharmaceutically acceptable salt, ester or prodrug thereof.

69. The composition of claim 46 wherein the HMG Co-A reductase inhibitor comprises fluvastatin, or a pharmaceutically acceptable salt, ester or prodrug thereof.

70. The composition of claim 46 wherein the HMG Co-A reductase inhibitor comprises ZD-4522, or a pharmaceutically acceptable salt, ester, conjugate acid, or prodrug thereof.

71. The composition of claim 46 wherein the HMG Co-A reductase inhibitor comprises NK-104, or a pharmaceutically acceptable salt, ester, conjugate acid, or prodrug thereof.

72. The composition of claim 46 wherein the apical sodium co-dependent bile acid transporter inhibitor comprises the racemate of 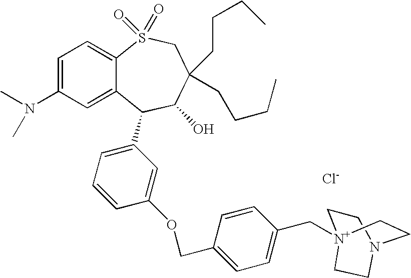
or a pharmaceutically acceptable salt, ester or prodrug thereof; and
the HMG Co-A reductase inhibitor is selected from the group consisting of mevastatin, lovastatin, simvastatin, pravastatin, fluvastatin, cerivastatin, atorvastatin, ZD-4522, NK-104, and the pharmaceutically acceptable salts, esters, conjugate acids, and prodrugs thereof.

73. The composition of claim 46 wherein the apical sodium co-dependent bile acid transporter inhibitor comprises the 4R,5R enantiomer of 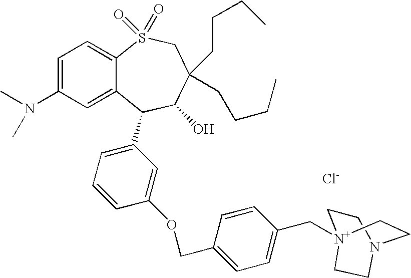
or a pharmaceutically acceptable salt, ester or prodrug thereof; and
the HMG Co-A reductase inhibitor is selected from the group consisting of mevastatin, lovastatin, simvastatin. pravastatin, fluvastatin, cerivastatin, atorvastatin, ZD-4522, NK-104, and the pharmaceutically acceptable salts, esters, conjugate acids, and prodrugs thereof.

74. The composition of claim 73 wherein the HMG Co-A reductase inhibitor is selected from the group consisting of atorvastatin, simvastatin, pravastatin, ZD-4522, NK-104, and the pharnaceutically acceptable salts. esters. conjugate acids. and prodrugs thereof.

75. The composition of claim 73 wherein the HMG Co-A reductase inhibitor comprises mevastatin, or a pharmaceutically acceptable salt, ester or prodrug thereof.

76. The composition of claim 73 wherein the HMG Co-A reductase inhibitor comprises lovastatin, or a pharmaceutically acceptable salt, ester or prodrug thereof.

77. The composition of claim 73 wherein the HMG Co-A reductase inhibitor comprises simvastatin, or a pharmaceutically acceptable salt, ester or prodrug thereof.

78. The composition of claim 73 wherein the HMG Co-A reductase inhibitor comprises pravastatin, or a pharmaceutically acceptable salt, ester or prodrug thereof.

79. The composition of claim 73 wherein the HMG Co-A reductase inhibitor comprises fluvastatin, or a pharmaceutically acceptable salt, ester or prodrug thereof.

80. The composition of claim 73 wherein the HMG Co-A reductase inhibitor comprises cerivastatin, or a pharmaceutically acceptable salt, ester or prodrug thereof.

81. The composition of claim 73 wherein the HMG Co-A reductase inhibitor comprises atorvastatin, or a pharmaceutically acceptable salt, ester or prodrug thereof.

82. The composition of claim 73 wherein the HMG Co-A reducase inhibitor comprises ZD-4522, or a pharmaceutically acceptable salt, ester, conjugate acid, or prodrug thereof.

83. The composition of claim 73 wherein the HMG Co-A reductase inhibitor comprises NK-104, or a pharmaceutically acceptable salt, ester, conjugate acid, or prodrug thereof.

84. The composition of claim 73 wherein the weight ratio of apical sodium codependent bile acid transporter inhibitor to HMG Co-A reductase inhibitor is between about 1:50 to about 3:1.

85. A kit containing a first dosage form comprising an ASBT inhibitor and a second dosage form comprising an HMG Co-A reductase inhibitor, wherein the apical sodium co-dependent bile acid transporter inhibitor is selected from the group consisting of: 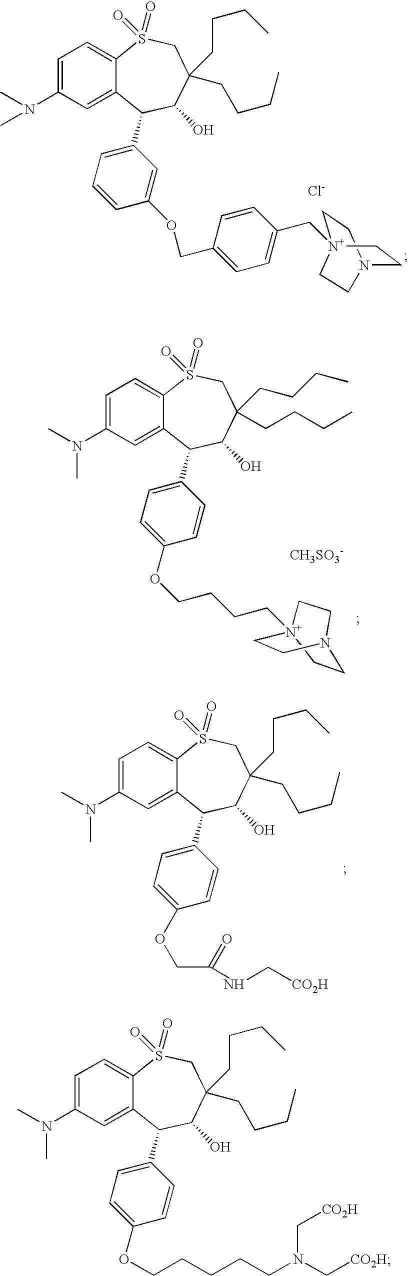 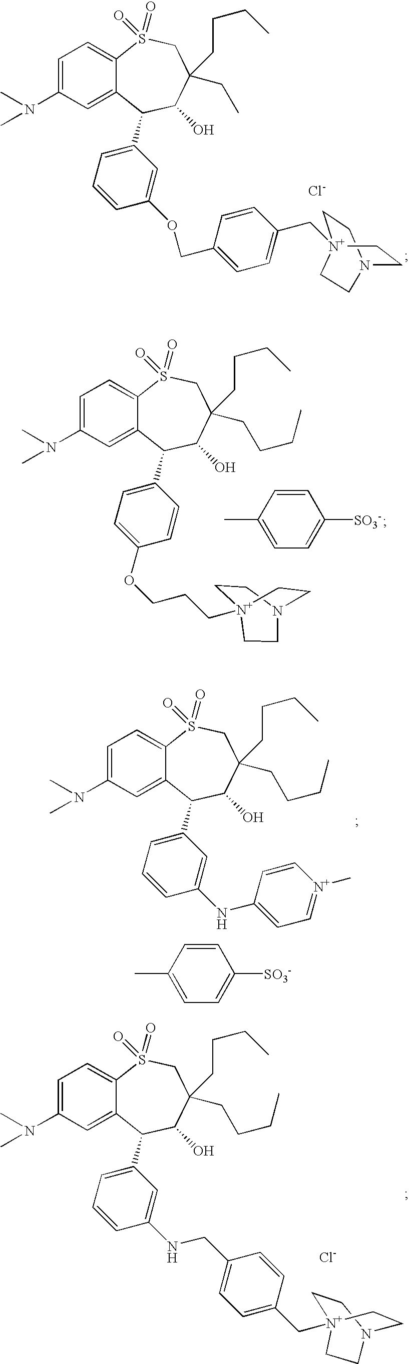 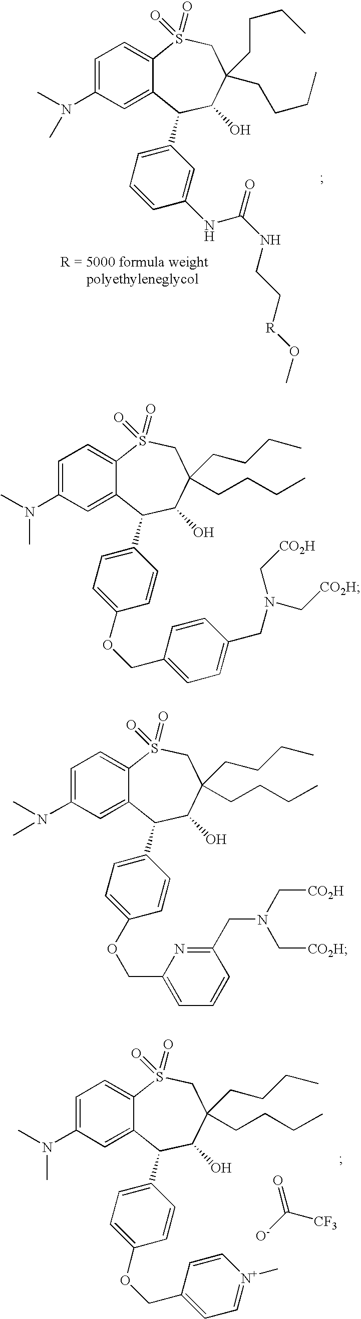 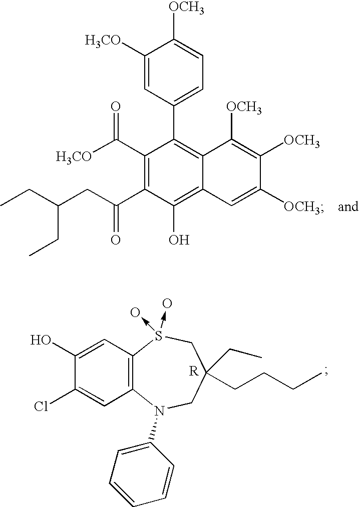
and the pharmaceutically acceptable salts, esters and prodrugs thereof.

86. A kit of claim 85 wherein the apical sodium co-dependent bile acid transporter inhibitor comprises the 4R,5R enantiomer of 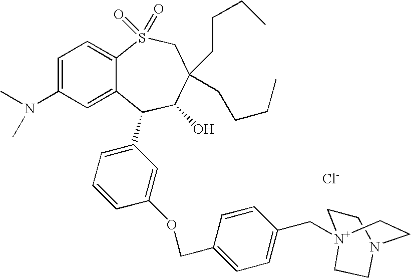
or a pharmaceutically acceptable salt, ester or prodrug thereof.

87. A kit of claim 86 wherein the HMG Co-A reductase inhibitor is selected from the group consisting of mevastatin, lovastatin, simvastatin, pravastatin, fluvastatin, cerivastatin, atorvastatin, ZD-4522, NK-104, and the pharmaceutically acceptable salts, esters, conjugate acids, and prodrugs thereof.

88. A kit of claim 86 wherein the HMG Co-A reductase inhibitor is selected from the group consisting of atorvastatin, simvastatin, pravastatin, ZD-4522, and the pharmaceutically acceptable salts, esters, conjugate acids, and prodrugs thereof.

89. The compound having the formula 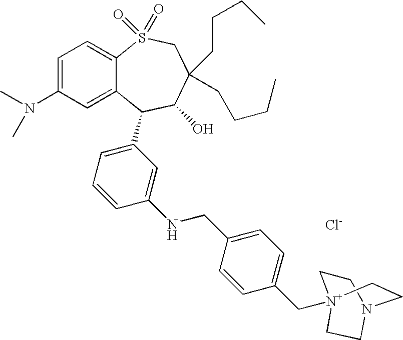
and the pharmaceutically acceptable salts, esters and prodrugs thereof.
